(19) United States
(12) Patent Application Publication
(10) Pub. No.: US 2024/0124907 A1
(43) Pub. Date: Apr. 18, 2024

(54) Engineered Biosynthetic Pathways for Production of Histamine by Fermentation

(71) Applicant: Zymergen Inc., Emeryville, CA (US)

(72) Inventors: Cara Ann TRACEWELL, Walnut Creek, CA (US); Alexander Glennon SHEARER, San Francisco, CA (US); Michael Shareef SIDDIQUI, Oakland, CA (US); Steven M. EDGAR, Albany, CA (US); Nicolaus HERMAN, El Cerrito, CA (US); Murtaza Shabbir HUSSAIN, Emeryville, CA (US)

(73) Assignee: Zymergen Inc., Emeryville, CA (US)

(21) Appl. No.: 18/349,895

(22) Filed: Jul. 10, 2023

Related U.S. Application Data
(63) Continuation of application No. 17/048,553, filed on Oct. 16, 2020, now Pat. No. 11,739,355.
(60) Provisional application No. 62/660,875, filed on Apr. 20, 2018.

(51) Int. Cl. C12P 17/10; C12N 1/18; C12N 1/20; C12N 9/04; C12N 9/10; C12N 9/88; C12N 15/77; C12N 15/81

(52) U.S. Cl. CPC C12P 17/10; C12N 1/18; C12N 1/20; C12N 9/0006; C12N 9/1077; C12N 9/88; C12N 15/77; C12N 15/81; C12Y 101/01023; C12Y 101/01049; C12Y 204/02017; C12Y 206/01009; C12Y 207/06001; C12Y 301/03015; C12Y 305/04019; C12Y 306/01031; C12Y 401/01022; C12Y 402/01019; C12Y 403/02; C12Y 503/01016

(57) ABSTRACT

The present disclosure describes the engineering of microbial cells for fermentative production of histamine and provides novel engineered microbial cells and cultures, as well as related histamine production methods.

### CROSS-REFERENCE TO RELATED APPLICATIONS

This application is a continuation of U.S. Non-provisional application Ser. No. 17/048,553, filed Oct. 16, 2020, which is a U.S. 371 National Phase of PCT International application no. PCT/US2019/028401, filed Apr. 19, 2019, which claims the benefit of U.S. provisional application No. 62/660,875, filed Apr. 20, 2018, each of which is hereby incorporated by reference in its entirety.

### STATEMENT AS TO RIGHTS TO INVENTIONS MADE UNDER FEDERALLY SPONSORED RESEARCH AND DEVELOPMENT

This invention was made with Government support under Agreement No. HR0011-15-9-0014, awarded by DARPA. The Government has certain rights in the invention.

### INCORPORATION BY REFERENCE OF THE SEQUENCE LISTING PROVIDED AS AN XML FILE

This application includes a sequence listing which has been submitted concurrently herewith as the sequence listing ST26 format XML file “ZGMNP011WO.xml”, file size 278,085 bytes, created on Jul. 10, 2023, and is hereby incorporated by reference in its entirety.

### FIELD OF THE DISCLOSURE

The present disclosure relates generally to the area of engineering microbes for production of histamine by fermentation.

### BACKGROUND

Biogenic amines are organic bases endowed with biological activity, which are frequently found in fermented foods and beverages. Histamine is known to exist in nature in fermented foods such as yogurt (13-36 mg/kg) [1], miso (24 mg/kg) [2], and red wine (24 mg/L) [3]. Some bacteria that live in the human gut also make histamine, and it functions to regulate the immune system by an anti-inflammatory effect [4]. Production of histamine in fermented foods relies on a source of proteins that contain histidine and microbes that histidine decarboxylase. Histamine is the decarboxylation product of histidine that is catalyzed specifically by the enzyme histidine decarboxylase (EC 4.1.1.22). Production of histamine in an industrial fermentation from simple, non-protein, carbon and nitrogen sources requires assembly of a pathway with improved biosynthesis of the amino acid precursor histidine and a highly active histidine decarboxylase.

### SUMMARY

The disclosure provides engineered microbial cells, cultures of the microbial cells, and methods for the production of histamine, including the following:

Embodiment 1: An engineered microbial cell that expresses a non-native histidine decarboxylase, wherein the engineered microbial cell produces histamine.

Embodiment 2: The engineered microbial cell of embodiment 1, wherein the engineered microbial cell includes increased activity of one or more upstream histamine pathway enzyme(s), said increased activity being increased relative to a control cell.

Embodiment 3: The engineered microbial cell of embodiment 2, wherein the one or more upstream histamine pathway enzyme(s) are selected from the group consisting of an ATP phosphoribosyltransferase, a phosphoribosyl-ATP pyrophosphatase, a phosphoribosyl-AMP cyclohydrolase, a 5′ProFAR isomerase, an imidazole-glycerol phosphate synthase, an imidazole-glycerol phosphate dehydratase, a histidinol-phosphate aminotransferase, a histidinol-phosphate phosphatase, histidinol dehydrogenase, and a ribose phosphate pyrophosphokinase.

Embodiment 4: The engineered microbial cell of any one of embodiments 1-3, wherein the engineered microbial cell includes reduced activity of one or more enzyme(s) that consume one or more histamine pathway precursors, said reduced activity being reduced relative to a control cell.

Embodiment 5: The engineered microbial cell of embodiment 4, wherein the one or more enzyme(s) that consume one or more histamine pathway precursors are selected from the group consisting of an enolase, a pyruvate dehydrogenase, a pentose phosphate pathway sugar isomerase, a transaldolase, a transketolase, a ribulose-5-phosphate epimerase, and a ribulose-5-phosphate isomerase.

Embodiment 6: The engineered microbial cell of embodiment 4 or embodiment 5, wherein the reduced activity is achieved by replacing a native promoter of a gene for said one or more enzymes with a less active promoter.

Embodiment 7: The engineered microbial cell of any one of embodiments 1-6, wherein the engineered microbial cell additionally expresses a feedback-deregulated glucose-6-phosphate dehydrogenase or a feedback-deregulated ATP phosphoribosyltransferase.

Embodiment 8: An engineered microbial cell, wherein the engineered microbial cell includes means for expressing a non-native histidine decarboxylase, wherein the engineered microbial cell produces histamine.

Embodiment 9: The engineered microbial cell of embodiment 8, wherein the engineered microbial cell includes means for increasing the activity of one or more upstream histamine pathway enzyme(s), said increased activity being increased relative to a control cell.

Embodiment 10: The engineered microbial cell of embodiment 9, wherein the one or more upstream histamine pathway enzyme(s) are selected from the group consisting of an ATP phosphoribosyltransferase, a phosphoribosyl-ATP pyrophosphatase, a phosphoribosyl-AMP cyclohydrolase, a 5′ProFAR isomerase, an imidazole-glycerol phosphate synthase, an imidazole-glycerol phosphate dehydratase, a histidinol-phosphate aminotransferase, a histidinol-phosphate phosphatase, a histidinol dehydrogenase, and a ribose phosphate pyrophosphokinase.

Embodiment 11: The engineered microbial cell of any one of embodiments 8-10, wherein the engineered microbial cell includes means for reducing the activity of one or more enzyme(s) that consume one or more histamine pathway precursors, said reduced activity being reduced relative to a control cell.

Embodiment 12: The engineered microbial cell of embodiment 11, wherein the one or more enzyme(s) that consume one or more histamine pathway precursors are selected from the group consisting of an enolase, a pyruvate dehydrogenase, pentose phosphate pathway sugar isomerase, a transketolase, a translaldolase, a ribulose-5-phosphate epimerase, and a ribulose-5-phosphate isomerase.

Embodiment 13: The engineered microbial cell of embodiment 11 or embodiment 12, wherein the reduced activity is achieved by means for replacing a native promoter of a gene for said one or more enzymes with a less active promoter.

Embodiment 14: The engineered microbial cell of any one of embodiments 8-13, wherein the engineered microbial cell additionally includes means for expressing glucose-6-phosphate dehydrogenase or a feedback-deregulated ATP phosphoribosyltransferase.

Embodiment 15: The engineered microbial cell of any one of embodiments 1-14, wherein the engineered microbial cell includes a fungal cell.

Embodiment 16: The engineered microbial cell of embodiment 15, wherein the engineered microbial cell includes a yeast cell.

Embodiment 17: The engineered microbial cell of embodiment 16, wherein the yeast cell is a cell of the genus Saccharomyces or Yarrowia.

Embodiment 18: The engineered microbial cell of embodiment 17, wherein the yeast cell is a cell of the genus Saccharomyces and of the species cerevisiae.

Embodiment 19: The engineered microbial cell of embodiment 17, wherein the yeast cell is a cell of the genus Yarrowia and of the species lipolytica.

Embodiment 20: The engineered microbial cell of any one of embodiments 1-19, wherein the non-native histidine decarboxylase includes a histidine decarboxylase having at least 70% amino acid sequence identity with a histidine decarboxylase from Chromobacterium sp. LK1 or from Acinetobacter baumannii strain AB0057.

Embodiment 21: The engineered microbial cell of any one of embodiments 1 and 16-20, wherein the engineered microbial cell includes increased activity of one or more upstream histamine pathway enzyme(s), said increased activity being increased relative to a control cell, wherein the one or more upstream histamine pathway enzyme(s) comprise an ATP phosphoribosyltransferase.

Embodiment 22: The engineered microbial cell of embodiment 21 wherein the increased activity of the ATP phosphoribosyltransferase is achieved by heterologously expressing it.

Embodiment 23: The engineered microbial cell of embodiment 22, wherein the heterologous ATP phosphoribosyltransferase has at least 70% amino acid sequence identity with an ATP phosphoribosyltransferase from S. cerevisiae.

Embodiment 24: The engineered microbial cell of any one of embodiments 16-23, wherein the engineered microbial cell includes a feedback-deregulated variant of a Corynebacterium glutamicum ATP phosphoribosyltransferase.

Embodiment 25: The engineered microbial cell of any one of embodiments 1-14, wherein the engineered microbial cell is a bacterial cell.

Embodiment 26: The engineered microbial cell of embodiment 25, wherein the bacterial cell is a cell of the genus Corynebacteria or Bacillus.

Embodiment 27: The engineered microbial cell of embodiment 26, wherein the bacterial cell is a cell of the genus Corynebacteria and of the species glutamicum.

Embodiment 28: The engineered microbial cell of embodiment 26, wherein the bacterial cell is a cell of the genus Bacillus and of the species subtilis.

Embodiment 29: The engineered microbial cell of any one of embodiments 25-28, wherein the non-native histidine decarboxylase includes a histidine decarboxylase having at least 70% amino acid sequence identity with a histidine decarboxylase from Acinetobacter baumannii or from Lactobacillus sp. (strain 30a).

Embodiment 30: The engineered microbial cell of any one of embodiments 1 and 25-29, wherein the engineered microbial cell includes increased activity of one or more upstream histamine pathway enzyme(s), said increased activity being increased relative to a control cell, wherein the one or more upstream histamine pathway enzyme(s) comprise an ATP phosphoribosyltransferase and an imidazole-glycerol phosphate dehydratase.

Embodiment 31: The engineered microbial cell of embodiment 30, wherein the increased activity of the ATP phosphoribosyltransferase or the imidazole-glycerol phosphate dehydratase is achieved by heterologously expressing it.

Embodiment 32: The engineered microbial cell of embodiment 31, wherein the heterologous ATP phosphoribosyltransferase has at least 70% amino acid sequence identity with an ATP phosphoribosyltransferase from Saccharomyces cerevisiae S288c or from Salmonella typhimurium LT2, or the heterologous imidazole-glycerol phosphate dehydratase has at least 70% amino acid sequence identity with an imidazole-glycerol phosphate dehydratase from Corynebacterium glutamicum.

Embodiment 33: The engineered microbial cell of any one of embodiments 25-32, wherein the engineered microbial cell includes a feedback-deregulated variant of a Salmonella typhimurium ATP phosphoribosyltransferase.

Embodiment 34: The engineered microbial cell of any one of embodiments 1-33, wherein, when cultured, the engineered microbial cell produces histamine at a level of at least 20 mg/L of culture medium.

Embodiment 35: The engineered microbial cell of embodiment 34, wherein, when cultured, the engineered microbial cell produces histamine at a level of at least 300 mg/L of culture medium.

Embodiment 36: A culture of engineered microbial cells according to any one of embodiments 1-35.

Embodiment 37: The culture of embodiment 36, wherein the engineered microbial cells are present in a concentration such that the culture has an optical density at 600 nm of 10-500.

Embodiment 38: The culture of any one of embodiments 36-37, wherein the culture includes histamine.

Embodiment 39: The culture of any one of embodiments 36-38, wherein the culture includes histamine at a level at least 20 mg/L of culture medium.

Embodiment 40: A method of culturing engineered microbial cells according to any one of embodiments 1-35, the method including culturing the cells under conditions suitable for producing histamine.

Embodiment 41: The method of embodiment 40, wherein the method includes fed-batch culture, with an initial glucose level in the range of 1-100 g/L, followed controlled sugar feeding.

Embodiment 42: The method of any one of embodiments 40-41, wherein the fermentation substrate includes glucose and a nitrogen source selected from the group consisting of urea, an ammonium salt, ammonia, and any combination thereof.

Embodiment 43: The method of any one of embodiments 40-42, wherein the culture is pH-controlled during culturing.

Embodiment 44: The method of any one of embodiments 40-43, wherein the culture is aerated during culturing.

Embodiment 45: The method of any one of embodiments 40-44, wherein the engineered microbial cells produce histamine at a level at least 20 mg/L of culture medium.

Embodiment 46: The method of any one of embodiments 40-45, wherein the method additionally includes recovering histamine from the culture.

Embodiment 47: A method for preparing histamine using microbial cells engineered to produce histamine, the method including: (a) expressing a non-native histidine decarboxylase in microbial cells; (b) cultivating the microbial cells in a suitable culture medium under conditions that permit the microbial cells to produce histamine, wherein the histamine is released into the culture medium; and isolating histamine from the culture medium.

### BRIEF DESCRIPTION OF THE DRAWINGS

FIG. 1: Biosynthetic pathway for histamine.

FIG. 2: Histamine titers measured in the extracellular broth following fermentation by the first-round engineered host Corynebacteria glutamicum. (See also Example 1, Table 1.)

FIG. 3: Histamine titers measured in the extracellular broth following fermentation by the first-round engineered host Saccharomyces cerevisiae. (See also Example 1, Table 1.)

FIG. 4: Histamine titers measured in the extracellular broth following fermentation by the second-round engineered host Corynebacteria glutamicum. (See also Example 1, Table 2.)

FIG. 5: Histamine titers measured in the extracellular broth following fermentation by the second-round engineered host Saccharomyces cerevisiae. (See also Example 1, Table 2.)

FIG. 6: Histamine titers measured in the extracellular broth following fermentation by the first-round engineered host Yarrowia lipolytica. (See also Example 2, Table 4.)

FIG. 7: Histamine titers measured in the extracellular broth following fermentation by the first-round engineered host Bacillus subtilis.

FIG. 8: Histamine acid titers measured in the extracellular broth following fermentation of Saccharomyces cerevisiae expressing the host evaluation designs.

FIG. 9: Histamine acid titers measured in the extracellular broth following fermentation of Corynebacteria glutamicum expressing the host evaluation designs.

FIG. 10: Histamine titers measured in the extracellular broth following fermentation by the third-round engineered host Saccharomyces cerevisiae. (Improvement round.)

FIG. 11: Integration of Promoter-Gene-Terminator into Saccharomyces cerevisiae and Yarrowia lipolytica.

FIG. 12: Promoter replacement in Saccharomyces cerevisiae and Yarrowia lipolytica.

FIG. 13: Targeted gene deletion in Saccharomyces cerevisiae and Yarrowia lipolytica.

FIG. 14: Integration of Promoter-Gene-Terminator into Corynebacteria glutamicum and Bacillus subtilis.

### DETAILED DESCRIPTION

This disclosure describes a method for the production of the small molecule histamine via fermentation by a microbial host from simple carbon and nitrogen sources, such as glucose and urea, respectively. This objective can be achieved by introducing a non-native metabolic pathway into a suitable microbial host for industrial fermentation of large-scale chemical products. Illustrative hosts include Saccharomyces cerevisiae, Yarrowia lypolytica, Corynebacteria glutamicum, and Bacillus subtilis. The engineered metabolic pathway links the central metabolism of the host to a non-native pathway to enable the production of histamine. The simplest embodiment of this approach is the expression of an enzyme, a non-native histidine decarboxylase enzyme, in a microbial host strain that can produce histidine. Further engineering of the metabolic pathway by modification of the microbial host central metabolism through overexpression and mutation of a key upstream pathway enzyme, ATP phosphoribosyltransferase, enabled titers of 505 mg/L histamine to be achieved.

The following disclosure describes how to engineer a microbe with the necessary characteristics to produce industrially feasible titers of histamine from simple carbon and nitrogen sources. Active histidine decarboxylases have been identified, and it has been found that feedback-deregulated ATP phosphoribosyltransferase and/or constitutive expression of native ATP phosphoribosyltransferase improve the titers of histidine by fermentation.

### Definitions

Terms used in the claims and specification are defined as set forth below unless otherwise specified.

The term “fermentation” is used herein to refer to a process whereby a microbial cell converts one or more substrate(s) into a desired product (such as histamine) by means of one or more biological conversion steps, without the need for any chemical conversion step.

The term “engineered” is used herein, with reference to a cell, to indicate that the cell contains at least one targeted genetic alteration introduced by man that distinguishes the engineered cell from the naturally occurring cell.

The term “native” is used herein to refer to a cellular component, such as a polynucleotide or polypeptide, that is naturally present in a particular cell. A native polynucleotide or polypeptide is endogenous to the cell.

When used with reference to a polynucleotide or polypeptide, the term “non-native” refers to a polynucleotide or polypeptide that is not naturally present in a particular cell.

When used with reference to the context in which a gene is expressed, the term “non-native” refers to a gene expressed in any context other than the genomic and cellular context in which it is naturally expressed. A gene expressed in a non-native manner may have the same nucleotide sequence as the corresponding gene in a host cell, but may be expressed from a vector or from an integration point in the genome that differs from the locus of the native gene.

The term “heterologous” is used herein to describe a polynucleotide or polypeptide introduced into a host cell. This term encompasses a polynucleotide or polypeptide, respectively, derived from a different organism, species, or strain than that of the host cell. In this case, the heterologous polynucleotide or polypeptide has a sequence that is different from any sequence(s) found in the same host cell. However, the term also encompasses a polynucleotide or polypeptide that has a sequence that is the same as a sequence found in the host cell, wherein the polynucleotide or polypeptide is present in a different context than the native sequence (e.g., a heterologous polynucleotide can be linked to a different promotor and inserted into a different genomic location than that of the native sequence). “Heterologous expression” thus encompasses expression of a sequence that is non-native to the host cell, as well as expression of a sequence that is native to the host cell in a non-native context.

As used with reference to polynucleotides or polypeptides, the term “wild-type” refers to any polynucleotide having a nucleotide sequence, or polypeptide having an amino acid, sequence present in a polynucleotide or polypeptide from a naturally occurring organism, regardless of the source of the molecule; i.e., the term “wild-type” refers to sequence characteristics, regardless of whether the molecule is purified from a natural source; expressed recombinantly, followed by purification; or synthesized. The term “wild-type” is also used to denote naturally occurring cells.

A “control cell” is a cell that is otherwise identical to an engineered cell being tested, including being of the same genus and species as the engineered cell, but lacks the specific genetic modification(s) being tested in the engineered cell.

Enzymes are identified herein by the reactions they catalyze and, unless otherwise indicated, refer to any polypeptide capable of catalyzing the identified reaction. Unless otherwise indicated, enzymes may be derived from any organism and may have a native or mutated amino acid sequence. As is well known, enzymes may have multiple functions and/or multiple names, sometimes depending on the source organism from which they derive. The enzyme names used herein encompass orthologs, including enzymes that may have one or more additional functions or a different name.

The term “feedback-deregulated” is used herein with reference to an enzyme that is normally negatively regulated by a downstream product of the enzymatic pathway (i.e., feedback-inhibition) in a particular cell. In this context, a “feedback-deregulated” enzyme is a form of the enzyme that is less sensitive to feedback-inhibition than the native enzyme native to the cell. A feedback-deregulated enzyme may be produced by introducing one or more mutations into a native enzyme. Alternatively, a feedback-deregulated enzyme may simply be a heterologous, native enzyme that, when introduced into a particular microbial cell, is not as sensitive to feedback-inhibition as the native, native enzyme. In some embodiments, the feedback-deregulated enzyme shows no feedback-inhibition in the microbial cell.

The term “histamine” refers to 2-(1I-Imidazol-4-yl)ethanamine (CAS #51-45-6).

The term “sequence identity,” in the context of two or more amino acid or nucleotide sequences, refers to two or more sequences that are the same or have a specified percentage of amino acid residues or nucleotides that are the same, when compared and aligned for maximum correspondence, as measured using a sequence comparison algorithm or by visual inspection.

For sequence comparison to determine percent nucleotide or amino acid sequence identity, typically one sequence acts as a “reference sequence,” to which a “test” sequence is compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence relative to the reference sequence, based on the designated program parameters. Alignment of sequences for comparison can be conducted using BLAST set to default parameters.

The term “titer,” as used herein, refers to the mass of a product (e.g., histamine) produced by a culture of microbial cells divided by the culture volume.

As used herein with respect to recovering histamine from a cell culture, “recovering” refers to separating the histamine from at least one other component of the cell culture medium.

### Engineering Microbes for Histamine Production

Histamine Biosynthesis Pathway

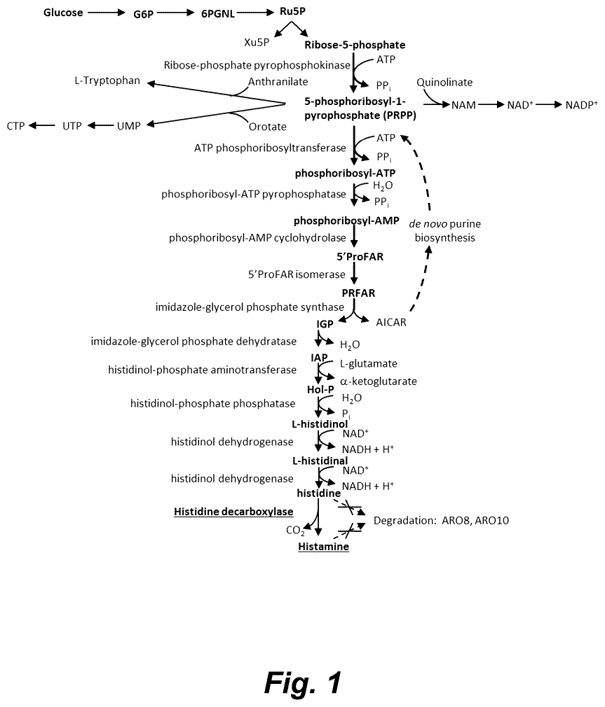

Histamine is typically derived from the amino acid histidine. The histamine biosynthesis pathway is shown in FIG. 1. The first enzyme of the amino acid biosynthesis pathway, ATP phosphoribosyltransferase, is subject to feedback inhibition by histidine. Histamine production is enabled by the addition of a single non-native enzymatic step in Saccharomyces cerevisiae, Yarrowia lypolytica, Corynebacteria glutamicum, and Bacillus subtilis hosts, which is catalyzed by histidine decarboxylase (EC 4.1.1.22).

Engineering for Microbial Histamine Production

Any histidine decarboxylase that is active in the microbial cell being engineered may be introduced into the cell, typically by introducing and expressing the gene(s) encoding the enzyme(s)s using standard genetic engineering techniques. Suitable histidine decarboxylase may be derived from any source, including plant, archaeal, fungal, gram-positive bacterial, and gram-negative bacterial sources. Exemplary sources include, but are not limited to: Aeromonas salmonicida subsp. pectinolytica 34mel, Acinetobacter baumannii (strain AB0057), Chromobacterium haemolyticum, Chromobacterium sp. LK1, Citrobacter pasteurii, Drosophila melanogaster, Lactobacillus aviarius DSM 20655, Lactobacillus fructivorans, Lactobacillus reuteri, Lactobacillus sp. (strain 30a), Methanosarcina barkeri (strain Fusaro/DSM804), Methanosarcina barkeri str. Wiesmoor, Morganella psychrotolerans, Mus musculus, Oenococcus oeni (Leuconostoc oenos), Pseudomonas putida (Arthrobacter siderocapsulatus), Pseudomonas rhizosphaerae, Pseudomonas sp. bs2935, Solanum lycopersicum, Oryza sativa, Penicillium marneffei, Streptomyces hygroscopicus, Pseudomonas putida, Arabidopsis thaliana (Mouse-ear cress), Glycine soja (Wild soybean), Solanum lycopersicum (Tomato) (Lycopersicon esculentum), Clostridium perfringens, Lactobacillus buchneri, Drosophila melanogaster (Fruitfly), Morganella morganii (Proteus morganii), E. coli, Bos taurus (Bovine), Raoutella planticol (Klebsiella planticola), Acinetobacter baumannii, Acinetobacter haemolyticus, Photobacterium damselae, Tetragenococcus muriaticus, Moritella sp JT01, Streptococcus thermophilus, Enterobacter aerogenes, Citrobacter youngae, Raoultella omithinolytica, and Raoultella planticola.

One or more copies of histidine decarboxylase gene can be introduced into a selected microbial host cell. If more than one copy of a gene is introduced, the copies can have the same or different nucleotide sequences. In some embodiments, one or both of the heterologous gene(s) is/are expressed from a strong, constitutive promoter. In some embodiments, the heterologous histidine decarboxylase gene(s) is/are expressed from an inducible promoter. The heterologous gene(s) can optionally be codon-optimized to enhance expression in the selected microbial host cell. Illustrative codon-optimization tables for hosts used in the Examples are as follows: Bacillus subtilis Kazusa codon table: www.kazusa.or.jp/codon/cgi-bin/showcodon.cgi?species=1423&aa=1&style=N; Yarrowia lipolytica Kazusa codon table: www.kazusa.or.jp/codon/cgi-bin/showcodon.cgi?species=4952&aa=1&style=N; Corynebacteria glutamicum Kazusa codon table: www.kazusa.or.jp/codon/cgi-bin/showcodon.cgi?species=340322&aa=1&style=N; Saccharomyces cerevisiae Kazusa codon table: http://www.kazusa.or.jp/codon/cgi-bin/showcodon.cgi?species=4932&aa=1&style=N. Also used, was a modified, combined codon usage scheme for S. cerevisae and C. glutamicum, which is reproduced below.

Modified Codon Usage Table for Sc and Cg

| Amino | | |
|---|---|---|
| Acid | Codon | Fraction |
| A | GCG | 0.22 |
| A | GCA | 0.29 |
| A | GCT | 0.24 |
| A | GCC | 0.25 |
| C | TGT | 0.36 |
| C | TGC | 0.64 |
| D | GAT | 0.56 |
| D | GAC | 0.44 |
| E | GAG | 0.44 |
| E | GM | 0.56 |
| F | TTT | 0.37 |
| F | TTC | 0.63 |
| G | GGG | 0.08 |
| G | GGA | 0.19 |
| G | GGT | 0.3 |
| G | GGC | 0.43 |
| H | CAT | 0.32 |
| H | CAC | 0.68 |
| I | ATA | 0.03 |
| I | ATT | 0.38 |
| I | ATC | 0.59 |
| K | MG | 0.6 |
| K | AAA | 0.4 |
| L | TTG | 0.29 |
| L | TTA | 0.05 |
| L | CTG | 0.29 |
| L | CTA | 0.06 |
| L | CTT | 0.17 |
| L | CTC | 0.14 |
| M | ATG | 1 |
| N | MT | 0.33 |
| N | MC | 0.67 |
| P | CCG | 0.22 |
| P | CCA | 0.35 |
| P | CCT | 0.23 |
| P | CCC | 0.2 |
| Q | CAG | 0.61 |
| Q | CM | 0.39 |
| R | AGG | 0.11 |
| R | AGA | 0.12 |
| R | CGG | 0.09 |
| R | CGA | 0.17 |
| R | CGT | 0.34 |
| R | CGC | 0.18 |
| S | AGT | 0.08 |
| S | AGC | 0.16 |
| S | TCG | 0.12 |
| S | TCA | 0.13 |
| S | TCT | 0.17 |
| S | TCC | 0.34 |
| T | ACG | 0.14 |
| T | ACA | 0.12 |
| T | ACT | 0.2 |
| T | ACC | 0.53 |
| V | GTG | 0.36 |
| V | GTA | 0.1 |
| V | GTT | 0.26 |
| V | GTC | 0.28 |
| W | TGG | 1 |
| Y | TAT | 0.34 |
| Y | TAC | 0.66 |

Increasing the Activity of Upstream Enzymes

One approach to increasing histamine production in a microbial cell that is capable of such production is to increase the activity of one or more upstream enzymes in the histamine biosynthesis pathway. Upstream pathway enzymes include all enzymes involved in the conversions from a feedstock all the way to into the last native metabolite (histidine, in the illustrative microbial cells described in the Examples below). Such enzymes include an ATP phosphoribosyltransferase, a phosphoribosyl-ATP pyrophosphatase, a phosphoribosyl-AMP cyclohydrolase, a 5′ProFAR isomerase, an imidazole-glycerol phosphate synthase, an imidazole-glycerol phosphate dehydratase, a histidinol-phosphate aminotransferase, a histidinol-phosphate phosphatase, histidinol dehydrogenase, and a ribose phosphate pyrophosphokinase. Suitable upstream pathway genes encoding these enzymes may be derived from any source, including, for example, those discussed above as sources for a histidine decarboxylase gene.

In some embodiments, the activity of one or more upstream pathway enzymes is increased by modulating the expression or activity of the native enzyme(s). For example, native regulators of the expression or activity of such enzymes can be exploited to increase the activity of suitable enzymes.

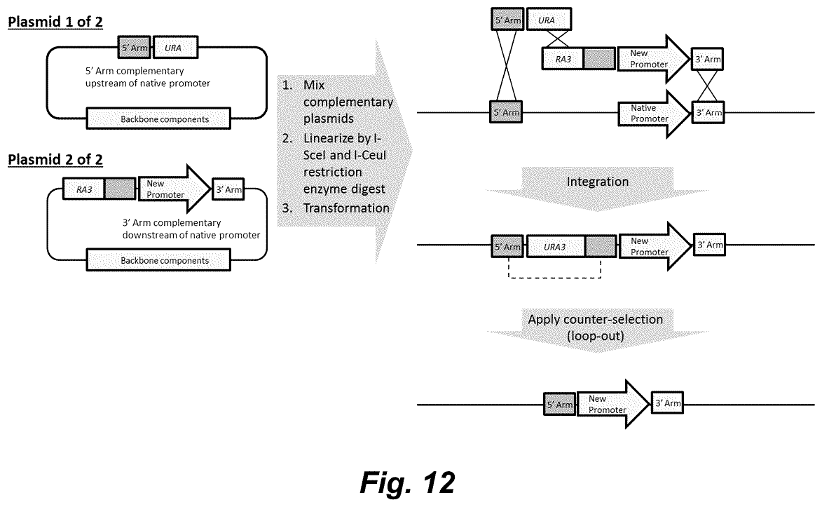

Alternatively, or in addition, one or more promoters can be substituted for native promoters using, for example, a technique such as that illustrated in FIG. 12. In certain embodiments, the replacement promoter is stronger than the native promoter and/or is a constitutive promoter.

In some embodiments, the activity of one or more upstream pathway enzymes is supplemented by introducing one or more of the corresponding genes into the histidine decarboxylase-expressing microbial host cell. An introduced upstream pathway gene may be from an organism other than that of the host cell or may simply be an additional copy of a native gene. In some embodiments, one or more such genes are introduced into a microbial host cell capable of histamine production and expressed from a strong constitutive promoter and/or can optionally be codon-optimized to enhance expression in the selected microbial host cell.

Example 1 describes the successful engineering of C. glutamicum to express a heterologous histamine decarboxylase from Acinetobacter baumannii (SEQ ID NO:1) and to constitutively express a heterologous C. glutamicum imidazoleglycerol-phosphate dehydratase (SEQ ID NO:2). This strain resulted from two rounds of genetic engineering and produced histamine at a titer of 24 mg/L of culture medium. This titer was increased to 68 mg/L in a C. glutamicum strain engineered to express a histamine decarboxylase from Acinetobacter baumannii (strain AB0057) (SEQ ID NO:1) and an ATP phosphoribosyltransferase from S. cerevisiae S288c (SEQ ID NO: 3).

Example 2 describes the successful engineering of Y. lypolytica to express a histidine decarboxylase from Acinetobacter baumannii (strain AB0057) (SEQ ID NO: 1) and an ATP phosphoribosyltransferase from S. cerevisiae S288c (SEQ ID NO:3) to give a histamine titer of 505 mg/L. Example 2 also describes the engineering B. subtilis to express a histamine decarboxylase from Lactobacillus sp. (strain 30a) (SEQ ID NO:4) and an ATP phosphoribosyltransferase from Salmonella typhimurium LT2 (SEQ ID NO:5) to give a histamine titer of 18 mg/L. Also in Example 2, S. cerevisiae was engineered to express a histamine decarboxylase from Chromobacterium sp. LK1 (SEQ ID NO:6) and an ATP phosphoribosyltransferase S. cerevisiae S288c (SEQ ID NO: 3) to give a histamine titer of 111 mg/L.

In various embodiments, the engineering of a histamine-producing microbial cell to increase the activity of one or more upstream pathway enzymes increases the histamine titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold. In various embodiments, the increase in histamine titer is in the range of 10 percent to 100-fold, 2-fold to 50-fold, 5-fold to 40-fold, 10-fold to 30-fold, or any range bounded by any of the values listed above. (Ranges herein include their endpoints.) These increases are determined relative to the histamine titer observed in a histamine-producing microbial cell that lacks any increase in activity of upstream pathway enzymes. This reference cell may have one or more other genetic alterations aimed at increasing histamine production, e.g., the cell may express a feedback-deregulated enzyme.

In various embodiments, the histamine titers achieved by increasing the activity of one or more upstream pathway genes are at least 1, 10, 20, 30, 40, 50, 75, 100, 200, 300, 400, 500, 600, 700, 800, or 900 mg/L or at least 1, 1.5, 2, 2.5, 3, 3.5, 4, 4.5, 5, or 10 gm/L. In various embodiments, the titer is in the range of 10 mg/L to 10 gm/L, 20 mg/L to 5 gm/L, 50 mg/L to 4 gm/L, 100 mg/L to 3 gm/L, 500 mg/L to 2 gm/L or any range bounded by any of the values listed above.

### Introduction of Feedback-Deregulated Enzymes

Since histidine biosynthesis is subject to feedback inhibition, another approach to increasing histamine production in a microbial cell engineered to produce histamine is to introduce feedback-deregulated forms of one or more enzymes that are normally subject to feedback regulation. Examples of such enzymes include glucose-6-phosphate dehydrogenase and ATP phosphoribosyltransferase. A feedback-deregulated form can be a heterologous, native enzyme that is less sensitive to feedback inhibition than the native enzyme in the particular microbial host cell. Alternatively, a feedback-deregulated form can be a variant of a native or heterologous enzyme that has one or more mutations or truncations rendering it less sensitive to feedback inhibition than the corresponding native enzyme. Examples of the latter include a variant ATP phosphoribosyltransferase (from C. glutamicum) containing the amino acid substitutions N215K, L231F, and T235A (SEQ ID NO:7) and a variant ATP phosphoribosyltransferase (from Salmonella typhimurium) containing the deletion of amino acids Q207 and E208 (SEQ ID NO:5).

In various embodiments, the engineering of a histamine-producing microbial cell to express a feedback-deregulated enzymes increases the histamine titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold. In various embodiments, the increase in histamine titer is in the range of 10 percent to 100-fold, 2-fold to 50-fold, 5-fold to 40-fold, 10-fold to 30-fold, or any range bounded by any of the values listed above. These increases are determined relative to the histamine titer observed in a histamine-producing microbial cell that does not express a feedback-deregulated enzyme. This reference cell may (but need not) have other genetic alterations aimed at increasing histamine production, i.e., the cell may have increased activity of an upstream pathway enzyme resulting from some means other than feedback-insensitivity.

In various embodiments, the histamine titers achieved by using a feedback-deregulated enzyme to increase flux though the histamine biosynthetic pathway are at least 100, 200, 300, 400, 500, 600, 700, 800, or 900 μg/L, or at least 1, 10, 50, 75, 100, 200, 300, 400, 500, 600, 700, 800, or 900 mg/L or at least 1, 1.5, 2, 2.5, 3, 3.5, 4, 4.5, 5, 10, 20, 50 g/L. In various embodiments, the titer is in the range of 50 μg/L to 50 g/L, 75 μg/L to 20 g/L, 100 μg/L to 10 g/L, 200 μg/L to 5 g/L, 500 μg/L to 4 g/L, 1 mg/L to 3 g/L, 500 mg/L to 2 g/L or any range bounded by any of the values listed above.

The approaches of supplementing the activity of one or more native enzymes and/or introducing one or more feedback-deregulated enzymes can be combined in histamine decarboxylase-expressing microbial cells to achieve even higher histamine production levels. For example, a histamine titer of 385 mg/L was achieved in S. cerevisiae in two rounds of engineering from the introduction of three genes: a histidine decarboxylase gene (from Chromobacterium sp. LK1) (SEQ ID NO:6), an ATP phosphoribosyltransferase (from C. glutamicum) containing the amino acid substitutions N215K, L231F, and T235A (SEQ ID NO:7), and a constitutively expressed ATP phosphoribosyltransferase from S. cerevisiae S288c (SEQ ID NO:3). (Example 1.)

### Reduction of Precursor Consumption

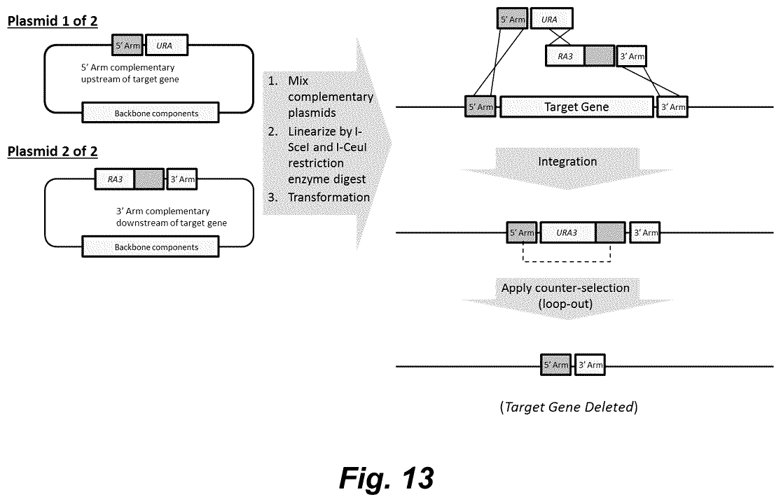

Another approach to increasing histamine production in a microbial cell that is capable of such production is to decrease the activity of one or more enzymes that consume one or more histamine pathway precursors. In some embodiments, the activity of one or more such enzymes is reduced by modulating the expression or activity of the native enzyme(s). Illustrative enzymes of this type include an enolase, a pyruvate dehydrogenase, a pentose phosphate pathway sugar isomerase, a transaldolase, a transketolase, a ribulose-5-phosphate epimerase, and a aribulose-5-phosphate isomerase. The activity of such enzymes can be decreased, for example, by substituting the native promoter of the corresponding gene(s) with a less active or inactive promoter or by deleting the corresponding gene(s). See FIGS. 12 and 13 for examples of schemes for promoter replacement and targeted gene deletion, respectively, in S. cerevisiae and Y. lipolytica.

In various embodiments, the engineering of a histamine-producing microbial cell to reduce precursor consumption by one or more side pathways increases the histamine titer by at least 10, 20, 30, 40, 50, 60, 70, 80, or 90 percent or by at least 2-fold, 2.5-fold, 3-fold, 3.5-fold, 4-fold, 4.5-fold, 5-fold, 5.5-fold, 6-fold, 6.5-fold, 7-fold, 7.5-fold, 8-fold, 8.5-fold, 9-fold, 9.5-fold, 10-fold, 11-fold, 12-fold, 13-fold, 14-fold, 15-fold, 16-fold, 17-fold, 18-fold, 19-fold, 20-fold, 21-fold, 22-fold, 23-fold, 24-fold, 25-fold, 30-fold, 35-fold, 40-fold, 45-fold, 50-fold, 55-fold, 60-fold, 65-fold, 70-fold, 75-fold, 80-fold, 85-fold, 90-fold, 95-fold, or 100-fold. In various embodiments, the increase in histamine titer is in the range of 10 percent to 100-fold, 2-fold to 50-fold, 5-fold to 40-fold, 10-fold to 30-fold, or any range bounded by any of the values listed above. These increases are determined relative to the histamine titer observed in a histamine-producing microbial cell that does not include genetic alterations to reduce precursor consumption. This reference cell may (but need not) have other genetic alterations aimed at increasing histamine production, i.e., the cell may have increased activity of an upstream pathway enzyme.

In various embodiments, the histamine titers achieved by reducing precursor consumption by one or more side pathways are at least 100, 200, 300, 400, 500, 600, 700, 800, or 900 μg/L, or at least 1, 10, 50, 75, 100, 200, 300, 400, 500, 600, 700, 800, or 900 mg/L or at least 1, 1.5, 2, 2.5, 3, 3.5, 4, 4.5, 5, 10, 20, 50 g/L. In various embodiments, the titer is in the range of 50 μg/L to 50 g/L, 75 μg/L to 20 g/L, 100 μg/L to 10 g/L, 200 μg/L to 5 g/L, 500 μg/L to 4 g/L, 1 mg/L to 3 g/L, 500 mg/L to 2 g/L or any range bounded by any of the values listed above.

The approaches of increasing the activity of one or more native enzymes and/or introducing one or more feedback-deregulated enzymes and/or reducing precursor consumption by one or more side pathways can be combined to achieve even higher histamine production levels.

Microbial Host Cells

Any microbe that can be used to express introduced genes can be engineered for fermentative production of histamine as described above. In certain embodiments, the microbe is one that is naturally incapable of fermentative production of histamine. In some embodiments, the microbe is one that is readily cultured, such as, for example, a microbe known to be useful as a host cell in fermentative production of compounds of interest. Bacteria cells, including gram positive or gram negative bacteria can be engineered as described above. Examples include, in addition to C. glutamicum cells, Bacillus subtilus, B. licheniformis, B. lentus, B. brevis, B. stearothermophilus, B. alkalophilus, B. amyloliquefaciens, B. clausii, B. halodurans, B. megaterium, B. coagulans, B. circulans, B. lautus, B. thuringiensis, S. albus, S. lividans, S. coelicolor, S. griseus, Pseudomonas sp., P. alcaligenes, P. citrea, Lactobacilis spp. (such as L. lactis, L. plantarum), L. grayi, E. coli, E. faecium, E. gallinarum, E. casseliflavus, and or E. faecalis cells.

There are numerous types of anaerobic cells that can be used as microbial host cells in the methods described herein. In some embodiments, the microbial cells are obligate anaerobic cells. Obligate anaerobes typically do not grow well, if at all, in conditions where oxygen is present. It is to be understood that a small amount of oxygen may be present, that is, there is some level of tolerance level that obligate anaerobes have for a low level of oxygen. Obligate anaerobes engineered as described above can be grown under substantially oxygen-free conditions, wherein the amount of oxygen present is not harmful to the growth, maintenance, and/or fermentation of the anaerobes.

Alternatively, the microbial host cells used in the methods described herein can be facultative anaerobic cells. Facultative anaerobes can generate cellular ATP by aerobic respiration (e.g., utilization of the TCA cycle) if oxygen is present. However, facultative anaerobes can also grow in the absence of oxygen. Facultative anaerobes engineered as described above can be grown under substantially oxygen-free conditions, wherein the amount of oxygen present is not harmful to the growth, maintenance, and/or fermentation of the anaerobes, or can be alternatively grown in the presence of greater amounts of oxygen.

In some embodiments, the microbial host cells used in the methods described herein are filamentous fungal cells. (See, e.g., Berka & Barnett, Biotechnology Advances, (1989), 7(2):127-154). Examples include Trichoderma longibrachiatum, T. viride, T. koningii, T. harzianum, Penicillum sp., Humicola insolens, H. lanuginose, H. grisea, Chrysosporium sp., C. lucknowense, Gliocladium sp., Aspergillus sp. (such as A. oryzae, A. niger, A. sojae, A. japonicus, A. nidulans, or A. awamori), Fusarium sp. (such as F. roseum, F. graminum F. cerealis, F. oxysporuim, or F. venenatum), Neurospora sp. (such as N. crassa or Hypocrea sp.), Mucor sp. (such as M. miehei), Rhizopus sp., and Emericella sp. cells. In particular embodiments, the fungal cell engineered as described above is A. nidulans, A. awamori, A. oryzae, A. aculeatus, A. niger, A. japonicus, T. reesei, T. viride, F. oxysporum, or F. solani. Illustrative plasmids or plasmid components for use with such hosts include those described in U.S. Patent Pub. No. 2011/0045563.

Yeasts can also be used as the microbial host cell in the methods described herein. Examples include: Saccharomyces sp., Schizosaccharomyces sp., Pichia sp., Hansenula polymorpha, Pichia stipites, Kluyveromyces marxianus, Kluyveromyces spp., Yarrowia lipolytica and Candida sp. In some embodiments, the Saccharomyces sp. is S. cerevisiae (See, e.g., Romanos et al., Yeast, (1992), 8(6):423-488). Illustrative plasmids or plasmid components for use with such hosts include those described in U.S. Pat. No. 7,659,097 and U.S. Patent Pub. No. 2011/0045563.

In some embodiments, the host cell can be an algal cell derived, e.g., from a green algae, red algae, a glaucophyte, a chlorarachniophyte, a euglenid, a chromista, or a dinoflagellate. (See, e.g., Saunders & Warmbrodt, “Gene Expression in Algae and Fungi, Including Yeast,” (1993), National Agricultural Library, Beltsville, Md.). Illustrative plasmids or plasmid components for use in algal cells include those described in U.S. Patent Pub. No. 2011/0045563.

In other embodiments, the host cell is a cyanobacterium, such as cyanobacterium classified into any of the following groups based on morphology: Chlorococcales, Pleurocapsales, Oscillatoriales, Nostocales, Synechosystic or Stigonematales (See, e.g., Lindberg et al., Metab. Eng., (2010) 12(1):70-79). Illustrative plasmids or plasmid components for use in cyanobacterial cells include those described in U.S. Patent Pub. Nos. 2010/0297749 and 2009/0282545 and in Intl. Pat. Pub. No. WO 2011/034863.

Genetic Engineering Methods

Microbial cells can be engineered for fermentative histamine production using conventional techniques of molecular biology (including recombinant techniques), microbiology, cell biology, and biochemistry, which are within the skill of the art. Such techniques are explained fully in the literature, see e.g., “Molecular Cloning: A Laboratory Manual,” fourth edition (Sambrook et al., 2012); “Oligonucleotide Synthesis” (M. J. Gait, ed., 1984); “Culture of Animal Cells: A Manual of Basic Technique and Specialized Applications” (R. I. Freshney, ed., 6th Edition, 2010); “Methods in Enzymology” (Academic Press, Inc.); “Current Protocols in Molecular Biology” (F. M. Ausubel et al., eds., 1987, and periodic updates); “PCR: The Polymerase Chain Reaction,” (Mullis et al., eds., 1994); Singleton et al., Dictionary of Microbiology and Molecular Biology 2nd ed., J. Wiley & Sons (New York, N.Y. 1994).

Vectors are polynucleotide vehicles used to introduce genetic material into a cell. Vectors useful in the methods described herein can be linear or circular. Vectors can integrate into a target genome of a host cell or replicate independently in a host cell. For many applications, integrating vectors that produced stable transformants are preferred. Vectors can include, for example, an origin of replication, a multiple cloning site (MCS), and/or a selectable marker. An expression vector typically includes an expression cassette containing regulatory elements that facilitate expression of a polynucleotide sequence (often a coding sequence) in a particular host cell. Vectors include, but are not limited to, integrating vectors, prokaryotic plasmids, episomes, viral vectors, cosmids, and artificial chromosomes.

Illustrative regulatory elements that may be used in expression cassettes include promoters, enhancers, internal ribosomal entry sites (IRES), and other expression control elements (e.g., transcription termination signals, such as polyadenylation signals and poly-U sequences). Such regulatory elements are described, for example, in Goeddel, Gene Expression Technology: Methods In Enzymology 185, Academic Press, San Diego, Calif. (1990).

In some embodiments, vectors may be used to introduce systems that can carry out genome editing, such as CRISPR systems. See U.S. Patent Pub. No. 2014/0068797, published 6 Mar. 2014; see also Jinek M., et al., “A programmable dual-RNA-guided DNA endonuclease in adaptive bacterial immunity,” Science 337:816-21, 2012). In Type II CRISPR-Cas9 systems, Cas9 is a site-directed endonuclease, namely an enzyme that is, or can be, directed to cleave a polynucleotide at a particular target sequence using two distinct endonuclease domains (HNH and RuvC/RNase H-like domains). Cas9 can be engineered to cleave DNA at any desired site because Cas9 is directed to its cleavage site by RNA. Cas9 is therefore also described as an “RNA-guided nuclease.” More specifically, Cas9 becomes associated with one or more RNA molecules, which guide Cas9 to a specific polynucleotide target based on hybridization of at least a portion of the RNA molecule(s) to a specific sequence in the target polynucleotide. Ran, F. A., et al., (“In vivo genome editing using Staphylococcus aureus Cas9,” Nature 520(7546):186-91, 2015, April 9], including all extended data) present the crRNA/tracrRNA sequences and secondary structures of eight Type II CRISPR-Cas9 systems. Cas9-like synthetic proteins are also known in the art (see U.S. Published Patent Application No. 2014-0315985, published 23 Oct. 2014).

Example 1 describes illustrative integration approaches for introducing polynucleotides and other genetic alterations into the genomes of C. glutamicum and S. cerevisiae cells.

Vectors or other polynucleotides can be introduced into microbial cells by any of a variety of standard methods, such as transformation, conjugation, electroporation, nuclear microinjection, transduction, transfection (e.g., lipofection mediated or DEAE-Dextrin mediated transfection or transfection using a recombinant phage virus), incubation with calcium phosphate DNA precipitate, high velocity bombardment with DNA-coated microprojectiles, and protoplast fusion. Transformants can be selected by any method known in the art. Suitable methods for selecting transformants are described in U.S. Patent Pub. Nos. 2009/0203102, 2010/0048964, and 2010/0003716, and International Publication Nos. WO 2009/076676, WO 2010/003007, and WO 2009/132220.

### Engineered Microbial Cells

The above-described methods can be used to produce engineered microbial cells that produce, and in certain embodiments, overproduce, histamine. Engineered microbial cells can have at least 1, 2, 3, 4, 5, 6, 7, 8, 9, 10, 20, 30, 40, 50, 60, 70, 80, 90, 100 or more genetic alterations, such as 30-100 alterations, as compared to a native microbial cell, such as any of the microbial host cells described herein. Engineered microbial cells described in the Example below have one, two, or three genetic alterations, but those of skill in the art can, following the guidance set forth herein, design microbial cells with additional alterations. In some embodiments, the engineered microbial cells have not more than 15, 14, 13, 12, 11, 10, 9, 8, 7, 6, 5, or 4 genetic alterations, as compared to a native microbial cell. In various embodiments, microbial cells engineered for histamine production can have a number of genetic alterations falling within the any of the following illustrative ranges: 1-10, 1-9, 1-8, 2-7, 2-6, 2-5, 2-4, 2-3, 3-7, 3-6, 3-5, 3-4, etc.

In some embodiments, an engineered microbial cell expresses at least one heterologous histamine decarboxylase, such as in the case of a microbial host cell that does not naturally produce histamine. In various embodiments, the microbial cell can include and express, for example: (1) a single heterologous histamine decarboxylase gene, (2) two or more heterologous histamine decarboxylase genes, which can be the same or different (in other words, multiple copies of the same heterologous histamine decarboxylase genes can be introduced or multiple, different heterologous histamine decarboxylase genes can be introduced), (3) a single heterologous histamine decarboxylase gene that is not native to the cell and one or more additional copies of an native histamine decarboxylase gene, or (4) two or more non-native histamine decarboxylase genes, which can be the same or different, and one or more additional copies of an native histamine decarboxylase gene.

This engineered host cell can include at least one additional genetic alteration that increases flux through the pathway leading to the production of histidine (the immediate precursor of histamine). These “upstream” enzymes in the pathway include: an ATP phosphoribosyltransferase, a phosphoribosyl-ATP pyrophosphatase, a phosphoribosyl-AMP cyclohydrolase, a 5′ProFAR isomerase, an imidazole-glycerol phosphate synthase, an imidazole-glycerol phosphate dehydratase, a histidinol-phosphate aminotransferase, a histidinol-phosphate phosphatase, histidinol dehydrogenase, and a ribose phosphate pyrophosphokinase, including any isoforms, paralogs, or orthologs having these enzymatic activities (which as those of skill in the art readily appreciate may be known by different names). The at least one additional alteration can increase the activity of the upstream pathway enzyme(s) by any available means, e.g., by: (1) modulating the expression or activity of the native enzyme(s), (2) expressing one or more additional copies of the genes for the native enzymes, and/or (3) expressing one or more copies of the genes for one or more non-native enzymes.

In some embodiments, increased flux through the pathway can be achieved by expressing one or more genes encoding a feedback-deregulated enzyme, as discussed above. For example, the engineered host cell can include and express one or more feedback-deregulated ATP phosphoribosyltransferase genes.

The engineered microbial cells can contain introduced genes that have a native nucleotide sequence or that differ from native. For example, the native nucleotide sequence can be codon-optimized for expression in a particular host cell. The amino acid sequences encoded by any of these introduced genes can be native or can differ from native. In various embodiments, the amino acid sequences have at least 60 percent, 70 percent, 75 percent, 80 percent, 85 percent, 90 percent, 95 percent or 100 percent amino acid sequence identity with a native amino acid sequence.

In some embodiments, increased availability of precursors to histamine can be achieved by reducing the expression or activity of enzymes that consume one or more histamine pathway precursors, such as an enolase, a pyruvate dehydrogenase, a pentose phosphate pathway sugar isomerase, a transaldolase, a transketolase, a ribulose-5-phosphate epimerase, and a aribulose-5-phosphate isomerase. For example, the engineered host cell can include one or more promoter swaps to down-regulate expression of any of these enzymes and/or can have their genes deleted to eliminate their expression entirely.

The approach described herein has been carried out in bacterial cells, namely C. glutamicum and B. subtilis (prokaryotes) and in fungal cells, namely the yeasts S. cerevisiae and Y. lypolytica (eukaryotes). (See Examples 1 and 2.)

Illustrative Engineered Yeast Cells

In certain embodiments, the engineered yeast (e.g., S. cerevisiae) cell expresses a heterologous histamine decarboxylase having at least 70 percent, 75 percent, 80 percent, 85 percent, 90 percent, 95 percent or 100 percent amino acid sequence identity to a histamine decarboxylase from Chromobacterium sp. LK1 (e.g., SEQ ID NO:6). In particular embodiments, the Chromobacterium sp. LK1 histamine decarboxylase can include SEQ ID NO:6. The engineered yeast (e.g., S. cerevisiae) cell can also express a heterologous ATP phosphoribosyltransferase having at least 70 percent, 75 percent, 80 percent, 85 percent, 90 percent, 95 percent or 100 percent amino acid sequence identity with an ATP phosphoribosyltransferase from S. cerevisiae (SEQ ID NO:3). In particular embodiments, the S. cerevisiae ATP phosphoribosyltransferase includes SEQ ID NO:3.

In certain embodiments, the engineered yeast (e.g., Y. lipolytica) cell expresses a heterologous histamine decarboxylase having at least 70 percent, 75 percent, 80 percent, 85 percent, 90 percent, 95 percent or 100 percent amino acid sequence identity to a histamine decarboxylase from Acinetobacter baumannii strain AB0057 (e.g., SEQ ID NO:1). In particular embodiments, the Acinetobacter baumannii strain AB0057 histamine decarboxylase can include SEQ ID NO:1. The engineered yeast (e.g., Y. lipolytica) cell can also express a heterologous ATP phosphoribosyltransferase having at least 70 percent, 75 percent, 80 percent, 85 percent, 90 percent, 95 percent or 100 percent amino acid sequence identity with an ATP phosphoribosyltransferase from S. cerevisiae S288c (SEQ ID NO:3). In particular embodiments, the S. cerevisiae S288c ATP phosphoribosyltransferase includes SEQ ID NO:3.

These may be the only genetic alterations of the engineered yeast cell, or the yeast cell can include one or more additional genetic alterations, as discussed more generally above.

For example, in particular embodiments, the engineered yeast S. cerevisiae cell described above additionally expresses a feedback deregulated variant of a C. glutamicum ATP phosphoribosyltransferase, which typically has at least 70 percent, 75 percent, 80 percent, 85 percent, 90 percent, or 95 percent amino acid sequence identity to a variant of a C. glutamicum ATP phosphoribosyltransferase containing the amino acid substitutions N215K, L231F, and T235A (SEQ ID NO:7) In particular embodiments, the C. glutamicum ATP phosphoribosyltransferase variant can include SEQ ID NO:7.

Illustrative Engineered Bacterial Cells

In certain embodiments, the engineered bacterial (e.g., C. glutamicum) cell expresses a heterologous histamine decarboxylase having at least 70 percent, 75 percent, 80 percent, 85 percent, 90 percent, 95 percent or 100 percent amino acid sequence identity to a histamine decarboxylase from Acinetobacter baumannii (e.g., SEQ ID NO:1). In particular embodiments, the Acinetobacter baumannii histamine decarboxylase can include SEQ ID NO:1. The engineered bacterial (e.g., C. glutamicum) cell can also express a heterologous ATP phosphoribosyltransferase having at least 70 percent, 75 percent, 80 percent, 85 percent, 90 percent, 95 percent or 100 percent amino acid sequence identity with an ATP phosphoribosyltransferase from Saccharomyces cerevisiae S288c (SEQ ID NO:3). In particular embodiments, the S. cerevisiae S288c ATP phosphoribosyltransferase includes SEQ ID NO:3. In some embodiments, the engineered bacterial (e.g., C. glutamicum) cell expresses, instead of the ATP phosphoribosyltransferase, an imidazole-glycerol phosphate dehydratase having at least 70 percent, 75 percent, 80 percent, 85 percent, 90 percent, 95 percent or 100 percent amino acid sequence identity to an imidazole-glycerol phosphate dehydratase from C. glutamicum (SEQ ID NO:2). In particular embodiments, the C. glutamicum imidazole-glycerol phosphate dehydratase includes SEQ ID NO:2.

In certain embodiments, the engineered bacterial (e.g., B. subtilis) cell expresses a heterologous histamine decarboxylase having at least 70 percent, 75 percent, 80 percent, 85 percent, 90 percent, 95 percent or 100 percent amino acid sequence identity to a histamine decarboxylase from Lactobacillus sp. (strain 30a) (e.g., SEQ ID NO:4). In particular embodiments, the Lactobacillus sp. (strain 30a) histamine decarboxylase can include SEQ ID NO:4. The engineered bacterial (e.g., B. subtilis) cell can also express a heterologous ATP phosphoribosyltransferase having at least 70 percent, 75 percent, 80 percent, 85 percent, 90 percent, 95 percent or 100 percent amino acid sequence identity with an ATP phosphoribosyltransferase from Salmonella typhimurium LT2 (SEQ ID NO:5). In particular embodiments, the Salmonella typhimurium LT2 ATP phosphoribosyltransferase includes SEQ ID NO:5.

### Culturing of Engineered Microbial Cells

Any of the microbial cells described herein can be cultured, e.g., for maintenance, growth, and/or histamine production.

In some embodiments, the cultures are grown to an optical density at 600 nm of 10-500, such as an optical density of 50-150.

In various embodiments, the cultures include produced histamine at titers of at least 10, 25, 50, 75, 100, 200, 300, 400, 500, 600, 700, 800, or 900 μg/L, or at least 1, 10, 50, 75, 100, 200, 300, 400, 500, 600, 700, 800, or 900 mg/L or at least 1, 1.5, 2, 2.5, 3, 3.5, 4, 4.5, 5, 10, 20, 50 g/L. In various embodiments, the titer is in the range of 10 μg/L to 10 g/L, 25 μg/L to 20 g/L, 100 μg/L to 10 g/L, 200 μg/L to 5 g/L, 500 μg/L to 4 g/L, 1 mg/L to 3 g/L, 500 mg/L to 2 g/L or any range bounded by any of the values listed above.

Culture Media

Microbial cells can be cultured in any suitable medium including, but not limited to, a minimal medium, i.e., one containing the minimum nutrients possible for cell growth. Minimal medium typically contains: (1) a carbon source for microbial growth; (2) salts, which may depend on the particular microbial cell and growing conditions; and (3) water. Suitable media can also include any combination of the following: a nitrogen source for growth and product formation, a sulfur source for growth, a phosphate source for growth, metal salts for growth, vitamins for growth, and other cofactors for growth.

Any suitable carbon source can be used to cultivate the host cells. The term “carbon source” refers to one or more carbon-containing compounds capable of being metabolized by a microbial cell. In various embodiments, the carbon source is a carbohydrate (such as a monosaccharide, a disaccharide, an oligosaccharide, or a polysaccharide), or an invert sugar (e.g., enzymatically treated sucrose syrup). Illustrative monosaccharides include glucose (dextrose), fructose (levulose), and galactose; illustrative oligosaccharides include dextran or glucan, and illustrative polysaccharides include starch and cellulose. Suitable sugars include C6 sugars (e.g., fructose, mannose, galactose, or glucose) and C5 sugars (e.g., xylose or arabinose). Other, less expensive carbon sources include sugar cane juice, beet juice, sorghum juice, and the like, any of which may, but need not be, fully or partially deionized.

The salts in a culture medium generally provide essential elements, such as magnesium, nitrogen, phosphorus, and sulfur to allow the cells to synthesize proteins and nucleic acids.

Minimal medium can be supplemented with one or more selective agents, such as antibiotics.

To produce histamine, the culture medium can include, and/or is supplemented during culture with, glucose and/or a nitrogen source such as urea, an ammonium salt, ammonia, or any combination thereof.

Culture Conditions

Materials and methods suitable for the maintenance and growth of microbial cells are well known in the art. See, for example, U.S. Pub. Nos. 2009/0203102, 2010/0003716, and 2010/0048964, and International Pub. Nos. WO 2004/033646, WO 2009/076676, WO 2009/132220, and WO 2010/003007, Manual of Methods for General Bacteriology Gerhardt et al., eds), American Society for Microbiology, Washington, D.C. (1994) or Brock in Biotechnology: A Textbook of Industrial Microbiology, Second Edition (1989) Sinauer Associates, Inc., Sunderland, Mass.

In general, cells are grown and maintained at an appropriate temperature, gas mixture, and pH (such as about 20° C. to about 37° C., about 6% to about 84% CO₂, and a pH between about 5 to about 9). In some aspects, cells are grown at 35° C. In certain embodiments, such as where thermophilic bacteria are used as the host cells, higher temperatures (e.g., 50° C.-75° C.) may be used. In some aspects, the pH ranges for fermentation are between about pH 5.0 to about pH 9.0 (such as about pH 6.0 to about pH 8.0 or about 6.5 to about 7.0). Cells can be grown under aerobic, anoxic, or anaerobic conditions based on the requirements of the particular cell.

Standard culture conditions and modes of fermentation, such as batch, fed-batch, or continuous fermentation that can be used are described in U.S. Publ. Nos. 2009/0203102, 2010/0003716, and 2010/0048964, and International Pub. Nos. WO 2009/076676, WO 2009/132220, and WO 2010/003007. Batch and Fed-Batch fermentations are common and well known in the art, and examples can be found in Brock, Biotechnology: A Textbook of Industrial Microbiology, Second Edition (1989) Sinauer Associates, Inc.

In some embodiments, the cells are cultured under limited sugar (e.g., glucose) conditions. In various embodiments, the amount of sugar that is added is less than or about 105% (such as about 100%, 90%, 80%, 70%, 60%, 50%, 40%, 30%, 20%, or 10%) of the amount of sugar that can be consumed by the cells. In particular embodiments, the amount of sugar that is added to the culture medium is approximately the same as the amount of sugar that is consumed by the cells during a specific period of time. In some embodiments, the rate of cell growth is controlled by limiting the amount of added sugar such that the cells grow at the rate that can be supported by the amount of sugar in the cell medium. In some embodiments, sugar does not accumulate during the time the cells are cultured. In various embodiments, the cells are cultured under limited sugar conditions for times greater than or about 1, 2, 3, 5, 10, 15, 20, 25, 30, 35, 40, 50, 60, or 70 hours or even up to about 5-10 days. In various embodiments, the cells are cultured under limited sugar conditions for greater than or about 5, 10, 15, 20, 25, 30, 35, 40, 50, 60, 70, 80, 90, 95, or 100% of the total length of time the cells are cultured. While not intending to be bound by any particular theory, it is believed that limited sugar conditions can allow more favorable regulation of the cells.

In some aspects, the cells are grown in batch culture. The cells can also be grown in fed-batch culture or in continuous culture. Additionally, the cells can be cultured in minimal medium, including, but not limited to, any of the minimal media described above. The minimal medium can be further supplemented with 1.0% (w/v) glucose (or any other six-carbon sugar) or less. Specifically, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose. In some cultures, significantly higher levels of sugar (e.g., glucose) are used, e.g., at least 10% (w/v), 20% (w/v), 30% (w/v), 40% (w/v), 50% (w/v), 60% (w/v), 70% (w/v), or up to the solubility limit for the sugar in the medium. In some embodiments, the sugar levels falls within a range of any two of the above values, e.g.: 0.1-10% (w/v), 1.0-20% (w/v), 10-70% (w/v), 20-60% (w/v), or 30-50% (w/v). Furthermore, different sugar levels can be used for different phases of culturing. For fed-batch culture (e.g., of S. cerevisiae or C. glutamicum), the sugar level can be about 100-200 g/L (10-20% (w/v)) in the batch phase and then up to about 500-700 g/L (50-70% in the feed).

Additionally, the minimal medium can be supplemented 0.1% (w/v) or less yeast extract. Specifically, the minimal medium can be supplemented with 0.1% (w/v), 0.09% (w/v), 0.08% (w/v), 0.07% (w/v), 0.06% (w/v), 0.05% (w/v), 0.04% (w/v), 0.03% (w/v), 0.02% (w/v), or 0.01% (w/v) yeast extract. Alternatively, the minimal medium can be supplemented with 1% (w/v), 0.9% (w/v), 0.8% (w/v), 0.7% (w/v), 0.6% (w/v), 0.5% (w/v), 0.4% (w/v), 0.3% (w/v), 0.2% (w/v), or 0.1% (w/v) glucose and with 0.1% (w/v), 0.09% (w/v), 0.08% (w/v), 0.07% (w/v), 0.06% (w/v), 0.05% (w/v), 0.04% (w/v), 0.03% (w/v), or 0.02% (w/v) yeast extract. In some cultures, significantly higher levels of yeast extract can be used, e.g., at least 1.5% (w/v), 2.0% (w/v), 2.5% (w/v), or 3% (w/v). In some cultures (e.g., of S. cerevisiae or C. glutamicum), the yeast extract level falls within a range of any two of the above values, e.g.: 0.5-3.0% (w/v), 1.0-2.5% (w/v), or 1.5-2.0% (w/v).

Illustrative materials and methods suitable for the maintenance and growth of the engineered microbial cells described herein can be found below in Example 1.

### Histamine Production and Recovery

Any of the methods described herein may further include a step of recovering histamine. In some embodiments, the produced histamine contained in a so-called harvest stream is recovered/harvested from the production vessel. The harvest stream may include, for instance, cell-free or cell-containing aqueous solution coming from the production vessel, which contains histamine as a result of the conversion of production substrate by the resting cells in the production vessel. Cells still present in the harvest stream may be separated from the histamine by any operations known in the art, such as for instance filtration, centrifugation, decantation, membrane crossflow ultrafiltration or microfiltration, tangential flow ultrafiltration or microfiltration or dead end filtration. After this cell separation operation, the harvest stream is essentially free of cells.

Further steps of separation and/or purification of the produced histamine from other components contained in the harvest stream, i.e., so-called downstream processing steps may optionally be carried out. These steps may include any means known to a skilled person, such as, for instance, concentration, extraction, crystallization, precipitation, adsorption, ion exchange, and/or chromatography. Any of these procedures can be used alone or in combination to purify histamine. Further purification steps can include one or more of, e.g., concentration, crystallization, precipitation, washing and drying, treatment with activated carbon, ion exchange, nanofiltration, and/or re-crystallization. The design of a suitable purification protocol may depend on the cells, the culture medium, the size of the culture, the production vessel, etc. and is within the level of skill in the art.

The following examples are given for the purpose of illustrating various embodiments of the disclosure and are not meant to limit the present disclosure in any fashion. Changes therein and other uses which are encompassed within the spirit of the disclosure, as defined by the scope of the claims, will be identifiable to those skilled in the art.

### Example 1—Construction and Selection of Strains of Corynebacteria glutamicum and Saccharomyces cerevisiae Engineered to Produce Histamine

Plasmid/DNA Design

All strains tested for this work were transformed with plasmid DNA designed using proprietary software. Plasmid designs were specific to each of the host organisms engineered in this work. The plasmid DNA was physically constructed by a standard DNA assembly method. This plasmid DNA was then used to integrate metabolic pathway inserts by one of two host-specific methods, each described below.

C. glutamicum Pathway Integration

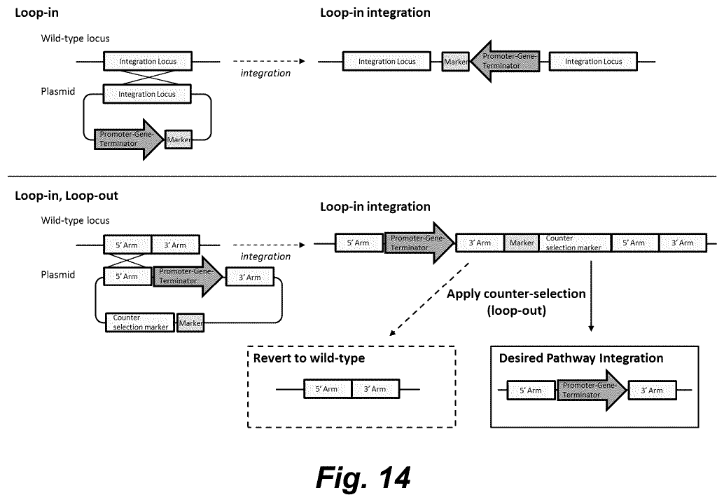

A “loop-in, single-crossover” genomic integration strategy has been developed to engineer C. glutamicum strains. FIG. 14 illustrates genomic integration of loop-in only and loop-in/loop-out constructs and verification of correct integration via colony PCR. Loop-in only constructs (shown under the heading “Loop-in”) contained a single 2-kb homology arm (denoted as “integration locus”), a positive selection marker (denoted as “Marker”)), and gene(s) of interest (denoted as “promoter-gene-terminator”). A single crossover event integrated the plasmid into the C. glutamicum chromosome. Integration events are stably maintained in the genome by growth in the presence of antibiotic (25 μg/ml kanamycin). Correct genomic integration in colonies derived from loop-in integration were confirmed by colony PCR with UF/IR and DR/IF PCR primers.

Loop-in, loop-out constructs (shown under the heading “Loop-in, loop-out) contained two 2-kb homology arms (5′ and 3′ arms), gene(s) of interest (arrows), a positive selection marker (denoted “Marker”), and a counter-selection marker. Similar to “loop-in” only constructs, a single crossover event integrated the plasmid into the chromosome of C. glutamicum. Note: only one of two possible integrations is shown here. Correct genomic integration was confirmed by colony PCR and counter-selection was applied so that the plasmid backbone and counter-selection marker could be excised. This results in one of two possibilities: reversion to wild-type (lower left box) or the desired pathway integration (lower right box). Again, correct genomic loop-out is confirmed by colony PCR. (Abbreviations: Primers: UF=upstream forward, DR=downstream reverse, IR=internal reverse, IF=internal forward.)

S. cerevisiae Pathway Integration

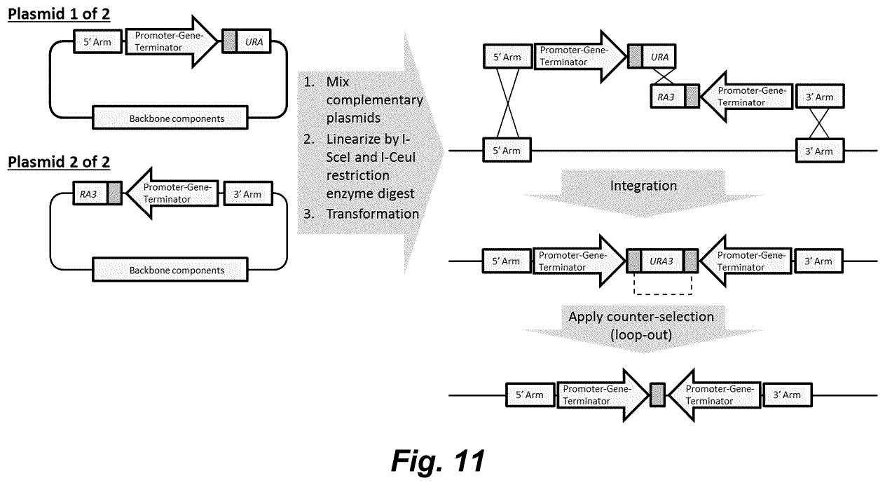

A “split-marker, double-crossover” genomic integration strategy has been developed to engineer S. cerevisiae strains. FIG. 11 illustrates genomic integration of complementary, split-marker plasmids and verification of correct genomic integration via colony PCR in S. cerevisiae. Two plasmids with complementary 5′ and 3′ homology arms and overlapping halves of a URA3 selectable marker (direct repeats shown by the hashed bars) were digested with meganucleases and transformed as linear fragments. A triple-crossover event integrated the desired heterologous genes into the targeted locus and re-constituted the full URA3 gene. Colonies derived from this integration event were assayed using two 3-primer reactions to confirm both the 5′ and 3′ junctions (UF/IF/wt-R and DR/IF/wt-F). For strains in which further engineering is desired, the strains can be plated on 5-FOA plates to select for the removal of URA3, leaving behind a small single copy of the original direct repeat. This genomic integration strategy can be used for gene knock-out, gene knock-in, and promoter titration in the same workflow.

Cell Culture

The workflow established for S. cerevisiae involved a hit-picking step that consolidated successfully built strains using an automated workflow that randomized strains across the plate. For each strain that was successfully built, up to four replicates were tested from distinct colonies to test colony-to-colony variation and other process variation. If fewer than four colonies were obtained, the existing colonies were replicated so that at least four wells were tested from each desired genotype.

The colonies were consolidated into 96-well plates with selective medium (SD-ura for S. cerevisiae) and cultivated for two days until saturation and then frozen with 16.6% glycerol at −80° C. for storage. The frozen glycerol stocks were then used to inoculate a seed stage in minimal media with a low level of amino acids to help with growth and recovery from freezing. The seed plates were grown at 30° C. for 1-2 days. The seed plates were then used to inoculate a main cultivation plate with minimal medium and grown for 48-88 hours. Plates were removed at the desired time points and tested for cell density (OD600), viability and glucose, supernatant samples stored for LC-MS analysis for product of interest.

Cell Density

Cell density was measured using a spectrophotometric assay detecting absorbance of each well at 600 nm. Robotics were used to transfer fixed amounts of culture from each cultivation plate into an assay plate, followed by mixing with 175 mM sodium phosphate (pH 7.0) to generate a 10-fold dilution. The assay plates were measured using a Tecan M1000 spectrophotometer and assay data uploaded to a LIMS database. A non-inoculated control was used to subtract background absorbance. Cell growth was monitored by inoculating multiple plates at each stage, and then sacrificing an entire plate at each time point.

To minimize settling of cells while handling large number of plates (which could result in a non-representative sample during measurement) each plate was shaken for 10-15 seconds before each read. Wide variations in cell density within a plate may also lead to absorbance measurements outside of the linear range of detection, resulting in underestimate of higher OD cultures. In general, the tested strains so far have not varied significantly enough for this be a concern.

Liquid-Solid Separation

To harvest extracellular samples for analysis by LC-MS, liquid and solid phases were separated via centrifugation. Cultivation plates were centrifuged at 2000 rpm for 4 minutes, and the supernatant was transferred to destination plates using robotics. 75 μL of supernatant was transferred to each plate, with one stored at 4° C., and the second stored at 80° C. for long-term storage.

First-Round Genetic Engineering Results in Corynebacteria glutamicum and Saccharomyces cerevisiae

A library approach was taken to screen heterologous pathway enzymes to establish the histamine pathway. For histidine decarboxylase, 18 heterologous sequences were tested from Bacteria, Archaea, Viridiplantae, Vertebrata, Metazoa, and Arthropoda sources listed in Table 1. The histidine decarboxylases were codon-optimized and expressed in both Saccharomyces cerevisiae and Corynebacteria glutamicum hosts.

Histidine biosynthesis is subject to feedback inhibition, therefore a feedback deregulated ATP phosphoribosyltransferase was tested with the histidine decarboxylases to improve production of histidine, the substrate for histidine decarboxylase. The ATP phosphoribosyltransferases tested were from Salmonella typhimurium and Corynebacteria glutamicum, harboring known deletions and point mutations that render them resistant to feedback inhibition.

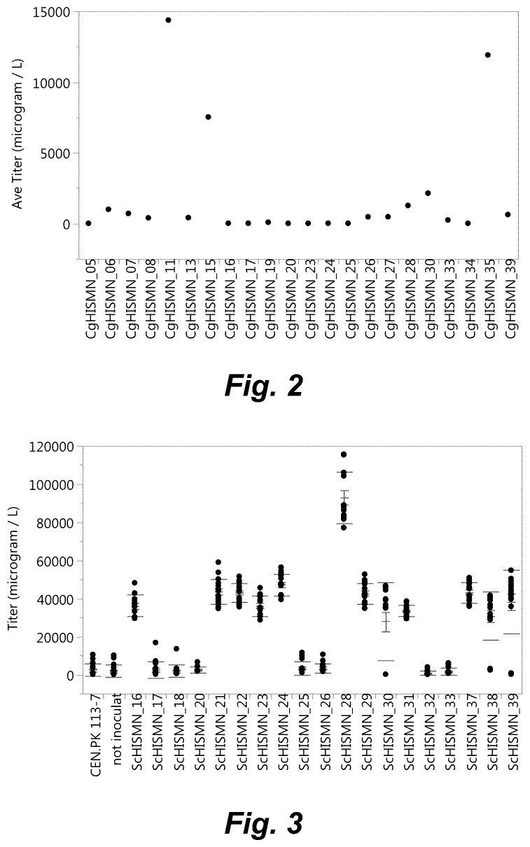

First-round genetic engineering results are shown in Table 1 and FIGS. 2 (C. glutamicum) and 3 (S. cerevisiae).

TABLE 1
First-round genetic engineering results in Corynebacteria glutamicum and Saccharomyces cerevisiae

| | | | | | | | | | | |
|---|---|---|---|---|---|---|---|---|---|---|
| | | | | | E1 | | | | | E2 |
| | | | Enzyme | Enzyme | Codon | | Enzyme | | Enzyme | Codon |
| | | E1 | 1- | 1- | Optimi- | E2 | 2- | E2 | 2- | Optimi- |
| Strain | Titer | Uniprot | activity | source | zation | Uniprot | activity | Modifi- | source | zation |
| name | (μg/L) | ID | name | organism | Abbrev. | ID | name | cations | organism | Abbrev. |

Corynebacterium glutamicum

| | | | | | | | | | | |
|---|---|---|---|---|---|---|---|---|---|---|
| CgHISMN_ | 985.0 | E3QMN8 | histidine | Methanosarcina | Cg | | | | | |
| 06 | | | decarboxylase | barkeri str. | | | | | | |
| | | | | Wiesmoor | | | | | | |
| CgHISMN_ | 695.7 | Q467R8 | histidine | Methanosarcina | Cg | | | | | |
| 07 | | | decarboxylase | barkeri (strain | | | | | | |
| | | | | Fusaro/ | | | | | | |
| | | | | DSM 804) | | | | | | |
| CgHISMN_ | 385.0 | P00862 | histidine | Lactobacillus sp. | Cg | | | | | |
| 08 | | | decarboxylase | (strain 30a) | | | | | | |
| CgHISMN_ | 14370.2 | B7I459 | histidine | Acinetobacter | Cg | | | | | |
| 11 | | | decarboxylase | baumannii | | | | | | |
| | | | | (strain | | | | | | |
| | | | | AB0057) | | | | | | |
| CgHISMN_ | 401.1 | E3QMN8 | histidine | Methanosarcina | Cg | | | | | |
| 13 | | | decarboxylase | barkeri str. | | | | | | |
| | | | | Wiesmoor | | | | | | |
| CgHISMN_ | 7529.8 | P00862 | histidine | Lactobacillus | Cg | P00499 | ATP | Deletion of | Salmonella | Cg |
| 15 | | | decarboxylase | sp. | | | phosphoribosyl- | Q207-E208 | typhimurium | |
| | | | | (strain 30a) | | | transferase | | | |
| CgHISMN_ | 4.0 | P00862 | histidine | Lactobacillus | Cg | P00499 | ATP | Deletion of | Salmonella | Cg |
| 16 | | | decarboxylase | sp. | | | phosphoribosyl- | Q207-E208 | typhimurium | |
| | | | | (strain 30a) | | | transferase | | | |
| CgHISMN_ | 3.9 | P23738 | histidine | Methanosarcina | Cg | P00499 | ATP | Deletion of | Salmonella | Cg |
| 17 | | | decarboxylase | barkeri (strain | | | phosphoribosyl- | Q207-E208 | typhimurium | |
| | | | | Fusaro/ | | | transferase | | | |
| | | | | DSM 804) | | | | | | |
| CgHISMN_ | 75.0 | Q05733 | histidine | Drosophila | Cg | P00499 | ATP | Deletion of | Salmonella | Cg |
| 19 | | | decarboxylase | melanogaster | | | phosphoribosyl- | Q207-E208 | typhimurium | |
| | | | | | | | transferase | | | |
| CgHISMN_ | 3.8 | J6KM89 | histidine | Chromo- | Cg | P00499 | ATP | Deletion of | Salmonella | Cg |
| 24 | | | decarboxylase | bacterium | | | phosphoribosyl- | Q207-E208 | typhimurium | |
| | | | | sp. LK1 | | | transferase | | | |
| CgHISMN_ | 1.7 | E3QMN8 | histidine | Methanosarcina | Cg | P00499 | ATP | Deletion of | Salmonella | Cg |
| 25 | | | decarboxylase | barkeri str. | | | phosphoribosyl- | Q207-E208 | typhimurium | |
| | | | | Wiesmoor | | | transferase | | | |
| CgHISMN_ | 458.5 | E3QMN8 | histidine | Methanosarcina | Cg | P00499 | ATP | Deletion of | Salmonella | Cg |
| 26 | | | decarboxylase | barkeri str. | | | phosphoribosyl- | Q207-E208 | typhimurium | |
| | | | | Wiesmoor | | | transferase | | | |
| CgHISMN_ | 462.1 | Q467R8 | histidine | Methanosarcina | Cg | P00499 | ATP | Deletion of | Salmonella | Cg |
| 27 | | | decarboxylase | barkeri (strain | | | phosphoribosyl- | Q207-E208 | typhimurium | |
| | | | | Fusaro/ | | | transferase | | | |
| | | | | DSM 804) | | | | | | |
| CgHISMN_ | 1258.2 | Q467R8 | histidine | Methanosarcina | Cg | P00499 | ATP | Deletion of | Salmonella | Cg |
| 28 | | | decarboxylase | barkeri (strain | | | phosphoribosyl- | Q207-E208 | typhimurium | |
| | | | | Fusaro/ | | | transferase | | | |
| | | | | DSM 804) | | | | | | |
| CgHISMN_ | 2126.4 | P00862 | histidine | Lactobacillus sp. | Cg | P00499 | ATP | Deletion of | Salmonella | Cg |
| 30 | | | decarboxylase | (strain 30a) | | | phosphoribosyl- | Q207-E208 | typhimurium | |
| | | | | | | | transferase | | | |
| CgHISMN_ | 234.7 | Q05733 | histidine | Drosophila | Cg | P00499 | ATP | Deletion of | Salmonella | Cg |
| 33 | | | decarboxylase | melanogaster | | | phosphoribosyl- | Q207-E208 | typhimurium | |
| | | | | | | | transferase | | | |
| CgHISMN_ | 11905.3 | B71459 | histidine | Acinetobacter | Cg | P00499 | ATP | Deletion of | Salmonella | Cg |
| 35 | | | decarboxylase | baumannii (strain | | | phosphoribosyl- | Q207-E208 | typhimurium | |
| | | | | AB0057) | | | transferase | | | |
| CgHISMN_ | 615.0 | E3QMN8 | histidine | Methanosarcina | Cg | P00499 | ATP | Deletion of | Salmonella | Cg |
| 39 | | | decarboxylase | barkeri str. | | | phosphoribosyl- | Q207-E208 | typhimurium | |
| | | | | Wiesmoor | | | transferase | | | |

Saccharomyces cerevisiae

| | | | | | | | | | | |
|---|---|---|---|---|---|---|---|---|---|---|
| ScHISMN_ | 36145.0 | P00862 | histidine | Lactobacillus | Sc | Q9Z472 | ATP | N215K, | Coryne- | Sc |
| 16 | | | decarboxylase | sp. | | | phosphoribosyl- | L231F, | bacterium | |
| | | | | (strain 30a) | | | transferase | T235A | glutamicum | |
| ScHISMN_ | 2369.9 | P54772 | histidine | Solanum | Sc | P00499 | ATP | Deletion of | Salmonella | Sc |
| 17 | | | decarboxylase | lycopersicum | | | phosphoribosyl- | Q207-E208 | typhimurium | |
| | | | | | | | transferase | | | |
| ScHISMN_ | 1747.7 | P23738 | histidine | Mus musculus | Sc | P00499 | ATP | Deletion of | Salmonella | Cg |
| 18 | | | decarboxylase | | | | phosphoribosyl- | Q207-E208 | typhimurium | |
| | | | | | | | transferase | | | |
| ScHISMN_ | 2432.4 | Q05733 | histidine | Drosophila | Sc | P00499 | ATP | Deletion of | Salmonella | Sc |
| 20 | | | decarboxylase | melanogaster | | | phosphoribosyl- | Q207-E208 | typhimurium | |
| | | | | | | | transferase | | | |
| ScHISMN_ | 43606.9 | J6KM89 | histidine | Chromo- | Sc | P00499 | ATP | Deletion of | Salmonella | Cg |
| 21 | | | decarboxylase | bacterium | | | phosphoribosyl- | Q207-E208 | typhimurium | |
| | | | | sp. LK1 | | | transferase | | | |
| ScHISMN_ | 43021.9 | E3QMN8 | histidine | Methanosarcina | Sc | Q9Z472 | ATP | N215K, | Coryne- | Sc |
| 22 | | | decarboxylase | barkeri str. | | | phosphoribosyl- | L231F, | bacterium | |
| | | | | Wiesmoor | | | transferase | T235A | glutamicum | |
| ScHISMN_ | 36145.8 | Q467R8 | histidine | Methanosarcina | Sc | P00499 | ATP | Deletion of | Salmonella | Sc |
| 23 | | | decarboxylase | barkeri (strain | | | phosphoribosyl- | Q207-E208 | typhimurium | |
| | | | | Fusaro/ | | | transferase | | | |
| | | | | DSM 804) | | | | | | |
| ScHISMN_ | 47208.0 | P00862 | histidine | Lactobacillus | Cg | P00499 | ATP | Deletion of | Salmonella | Cg |
| 24 | | | decarboxylase | sp. | | | phosphoribosyl- | Q207-E208 | typhimurium | |
| | | | | (strain 30a) | | | transferase | | | |
| ScHISMN_ | 3130.1 | P23738 | histidine | Mus musculus | Cg | Q9Z472 | ATP | N215K, | Coryne- | Sc |
| 25 | | | decarboxylase | | | | phosphoribosyl- | L231F, | bacterium | |
| | | | | | | | transferase | T235A | glutamicum | |
| ScHISMN_ | 3262.5 | Q05733 | histidine | Drosophila | Cg | P00499 | ATP | Deletion of | Salmonella | Sc |
| 26 | | | decarboxylase | melanogaster | | | phosphoribosyl- | Q207-E208 | typhimurium | |
| | | | | | | | transferase | | | |
| ScHISMN_ | 90811.0 | J6KM89 | histidine | Chromo- | Cg | Q9Z472 | ATP | N215K, | Coryne- | Sc |
| 28 | | | decarboxylase | bacterium | | | phosphoribosyl- | L231F, T235A | bacterium | |
| | | | | sp. LK1 | | | transferase | | glutamicum | |
| ScHISMN_ | 42708.8 | E3QMN8 | histidine | Methanosarcina | Cg | P00499 | ATP | Deletion of | Salmonella | Sc |
| 29 | | | decarboxylase | barkeri str. | | | phosphoribosyl- | Q207-E208 | typhimurium | |
| | | | | Wiesmoor | | | transferase | | | |
| ScHISMN_ | 27660.1 | Q467R8 | histidine | Methanosarcina | Cg | P00499 | ATP | Deletion of | Salmonella | Cg |
| 30 | | | decarboxylase | barkeri | | | phosphoribosyl- | Q207-E208 | typhimurium | |
| | | | | (strain | | | transferase | | | |
| | | | | Fusaro/ | | | | | | |
| | | | | DSM 804) | | | | | | |
| ScHISMN_ | 33356.6 | P00862 | histidine | Lactobacillus | Sc | Q9Z472 | ATP | N215K, | Coryne- | Sc |
| 31 | | | decarboxylase | sp. | | | phosphoribosyl- | L231F, | bacterium | |
| | | | | (strain 30a) | | | transferase | T235A | glutamicum | |
| ScHISMN_ | 711.5 | P54772 | histidine | Solanum | Sc | P00499 | ATP | Deletion of | Salmonella | Sc |
| 32 | | | decarboxylase | lycopersicum | | | phosphoribosyl- | Q207-E208 | typhimurium | |
| | | | | | | | transferase | | | |
| ScHISMN_ | 1523.1 | P23738 | histidine | Mus musculus | Sc | P00499 | ATP | Deletion of | Salmonella | Cg |
| 33 | | | decarboxylase | | | | phosphoribosyl- | Q207-E208 | typhimurium | |
| | | | | | | | transferase | | | |
| ScHISMN_ | 43170.7 | E3QMN8 | histidine | Methanosarcina | Sc | Q9Z472 | ATP | N215K, | Coryne- | Sc |
| 37 | | | decarboxylase | barkeri str. | | | phosphoribosyl- | L231F, | bacterium | |
| | | | | Wiesmoor | | | transferase | T235A | glutamicum | |
| ScHISMN_ | 30675.5 | Q467R8 | histidine | Methanosarcina | Sc | P00499 | ATP | Deletion of | Salmonella | Sc |
| 38 | | | decarboxylase | barkeri (strain | | | phosphoribosyl- | Q207-E208 | typhimurium | |
| | | | | Fusaro/ | | | transferase | | | |
| | | | | DSM 804) | | | | | | |
| ScHISMN_ | 38293.2 | P00862 | histidine | Lactobacillus | Cg | P00499 | ATP | Deletion of | Salmonella | Cg |
| 39 | | | decarboxylase | sp. | | | phosphoribosyl- | Q207-E208 | typhimurium | |
| | | | | (strain 30a) | | | transferase | | | |

Note:
“Cg” refers to codon optimization for Corynebacterium glutamicum; “Sc” refers to codon optimization for Saccharomyces cerevisiae.

Second-Round Genetic Engineering Results in Corynebacteria glutamicum and Saccharomyces cerevisiae

A library approach was taken to improve histamine production by separately expressing each upstream pathway enzyme with a constitutive promoter to screen for the rate-limiting step. The histidine pathway enzymes screened are listed in Table 2. In addition, the enzymes in Table 2, the strains contained the best enzymes from first round: the Corynebacteria glutamicum host contains histidine decarboxylase (UniProt ID B71459) (SEQ ID NO: 1) and ATP phosphoribosyltransferase (UniProt ID P00499) (SEQ ID NO: 5) containing the deletion Q207-E208, and the Saccharomyces cerevisiae host contains histidine decarboxylase (UniProt ID J6KM89)(SEQ ID NO: 6) and ATP phosphoribosyltransferase (UniProt ID Q9Z472) (SEQ ID NO: 7) containing the amino acid substitutions N215K, L231F and T235A.

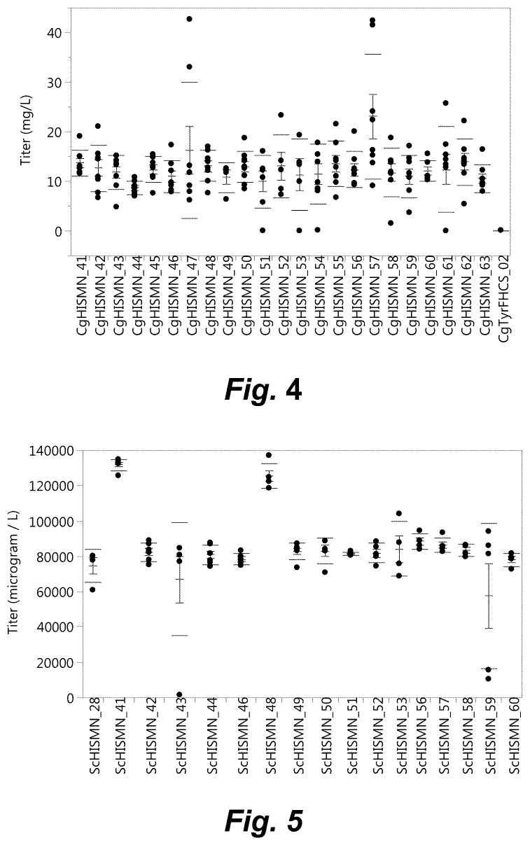

Second-round genetic engineering results are shown in Table 2 and FIGS. 4 (C. glutamicum) and 5 (S. cerevisiae).

In C. glutamicum, a titer of 24 mg/L was achieved after two rounds of engineering from the integration of two genes: a histidine decarboxylase gene from Acinetobacter baumannii, and constitutive expression of an imidazoleglycerol-phosphate dehydratase from C. glutamicum.

In S. cerevisiae, a titer of 385 mg/L was achieved in two rounds of engineering from the integration of three genes: a histidine decarboxylase gene from Chromobacterium sp. LK1 (SEQ ID NO: 6), an ATP phosphoribosyltransferase from C. glutamicum containing the amino acid substitutions N215K, L23IF, and T235A (SEQ ID NO: 7), and a constitutively expressed ATP phosphoribosyltransferase from S. cerevisiae (SEQ ID NO: 3).

TABLE 2
Second-round genetic engineering results in genetic engineering results in
Corynebacteria glutamicum and Saccharomyces cerevisiae

| | | | | | E1 Codon |
|---|---|---|---|---|---|
| | Titer | E1 | | Enzyme 1- | Optimization |
| Strain name | (μg/L) | Uniprot ID | Enzyme 1-activity name | source organism | Abbrev. |
| Corynebacteria | | | | | |
| glutamicum | | | | | |
| CgHISMN_41 | 13702.1 | O68602 | 1-(5-phosphoribosyl)5[(5- | Corynebacterium | Native |
| | | | phosphoribosylamino) | glutamicum | |
| | | | methylideneamino] | | |
| | | | imidazole-4- | | |
| | | | carboxamide isomerase | | |
| CgHISMN_42 | 12671.2 | Q9KJU3 | Imidazoleglycerol- | Corynebacterium | Native |
| | | | phosphate dehydratase | glutamicum | |
| CgHISMN_43 | 11800.4 | Q9KJU4 | Histidinol-phosphate | Corynebacterium | Native |
| | | | aminotransferase | glutamicum | |
| CgHISMN_44 | 8667.2 | Q8NNT5 | Histidinol dehydrogenase | Corynebacterium | Native |
| | | | | glutamicum | |
| CgHISMN_45 | 12375.3 | Q9Z471 | Phosphoribosyl- | Corynebacterium | Native |
| | | | ATP pyrophosphatase | glutamicum | |
| CgHISMN_46 | 10963.6 | O31139 | Imidazole glycerol | Corynebacterium | Native |
| | | | phosphate synthase subunit | glutamicum | |
| CgHISMN_47 | 16246.0 | O69043 | Imidazole glycerol | Corynebacterium | Native |
| | | | phosphate synthase subunit | glutamicum | |
| CgHISMN_48 | 13038.8 | Q9Z472 | ATP phosphoribosyltransferase | Corynebacterium | Native |
| | | | | glutamicum | |
| CgHISMN_49 | 10749.0 | Q8NNT9 | phosphoribosyl- | Corynebacterium | Native |
| | | | AMP cyclohydrolase | glutamicum | |
| CgHISMN_50 | 12960.8 | O68602 | 1-(5-phosphoribosyl)5[(5- | Corynebacterium | Native |
| | | | phosphoribosylamino) | glutamicum | |
| | | | methylideneamino] | | |
| | | | imidazole-4- | | |
| | | | carboxamide isomerase | | |
| CgHISMN_51 | 9958.4 | Q9KJU3 | Imidazoleglycerol- | Corynebacterium | Native |
| | | | phosphate dehydratase | glutamicum | |
| CgHISMN_52 | 18963.0 | Q9KJU4 | Histidinol-phosphate | Corynebacterium | Native |
| | | | aminotransferase | glutamicum | |
| CgHISMN_53 | 20328.9 | Q8NNT5 | Histidinol dehydrogenase | Corynebacterium | Native |
| | | | | glutamicum | |
| CgHISMN_54 | 20051.4 | O31139 | Imidazole glycerol phosphate | Corynebacterium | Native |
| | | | synthase subunit | glutamicum | |
| CgHISMN_55 | 15070.9 | O69043 | Imidazole glycerol phosphate | Corynebacterium | Native |
| | | | synthase subunit | glutamicum | |
| CgHISMN_56 | 12799.1 | O68602 | 1-(5-phosphoribosyl)5[(5- | Corynebacterium | Native |
| | | | phosphoribosylamino) | glutamicum | |
| | | | methylideneamino] | | |
| | | | imidazole-4- | | |
| | | | carboxamide isomerase | | |
| CgHISMN_57 | 24773.6 | Q9KJU3 | Imidazoleglycerol- | Corynebacterium | Native |
| | | | phosphate dehydratase | glutamicum | |
| CgHISMN_58 | 15268.6 | Q9KJU4 | Histidinol-phosphate | Corynebacterium | Native |
| | | | aminotransferase | glutamicum | |
| CgHISMN_59 | 12555.0 | Q8NNT5 | Histidinol dehydrogenase | Corynebacterium | Native |
| | | | | glutamicum | |
| CgHISMN_60 | 17725.6 | Q9Z471 | Phosphoribosyl- | Corynebacterium | Native |
| | | | ATP pyrophosphatase | glutamicum | |
| CgHISMN_61 | 18777.4 | O69043 | Imidazole glycerol | Corynebacterium | Native |
| | | | phosphate synthase subunit | glutamicum | |
| CgHISMN_62 | 19782.8 | Q9Z472 | ATP phosphoribosyltransferase | Corynebacterium | Native |
| | | | | glutamicum | |
| CgHISMN_63 | 15092.7 | Q8NNT9 | phosphoribosyl- | Corynebacterium | Native |
| | | | AMP cyclohydrolase | glutamicum | |
| Saccharomyces | | | | | |
| cerevisiae | | | | | |
| ScHISMN_41 | 385518.2 | P00498 | ATP phosphoribosyltransferase | Saccharomyces | Native |
| | | | | cerevisiae | |
| ScHISMN_42 | 70003.1 | P00815 | histidinol dehydrogenase, | Saccharomyces | Native |
| | | | phosphoribosyl-AMP cyclohydrolase, | cerevisiae | |
| | | | phosphoribosyl-ATP diphosphatase | | |
| ScHISMN_43 | 75039.5 | P33734 | Imidazole glycerol phosphate | Saccharomyces | Native |
| | | | synthase subunit HisF | cerevisiae | |
| ScHISMN_44 | 71402.5 | P07172 | histidinol-phosphate transaminase | Saccharomyces | Native |
| | | | | cerevisiae | |
| ScHISMN_46 | 64866.5 | P06633 | Imidazoleglycerol- | Saccharomyces | Native |
| | | | phosphate dehydratase | cerevisiae | |
| ScHISMN_48 | 113026.6 | P00498 | ATP phosphoribosyltransferase | Saccharomyces | Native |
| | | | | cerevisiae | |
| ScHISMN_49 | 79488.5 | P00815 | histidinol dehydrogenase, | Saccharomyces | Native |
| | | | phosphoribosyl-AMP cyclohydrolase, | cerevisiae | |
| | | | phosphoribosyl-ATP diphosphatase | | |
| ScHISMN_50 | 92719.6 | P33734 | Imidazole glycerol phosphate | Saccharomyces | Native |
| | | | synthase subunit HisF | cerevisiae | |
| ScHISMN_51 | 88847.1 | P07172 | histidinol-phosphate transaminase | Saccharomyces | Native |
| | | | | cerevisiae | |
| ScHISMN_52 | 70650.9 | P38635 | histidinol-phosphatase | Saccharomyces | Native |
| | | | | cerevisiae | |
| ScHISMN_53 | 74127.8 | P06633 | Imidazoleglycerol- | Saccharomyces | Native |
| | | | phosphate dehydratase | cerevisiae | |
| ScHISMN_56 | 73080.2 | P00815 | histidinol dehydrogenase, | Saccharomyces | Native |
| | | | phosphoribosyl-AMP cyclohydrolase, | cerevisiae | |
| | | | phosphoribosyl-ATP diphosphatase | | |
| ScHISMN_57 | 78656.1 | P33734 | Imidazole glycerol phosphate | Saccharomyces | Native |
| | | | synthase subunit HisF | cerevisiae | |
| ScHISMN_58 | 69769.0 | P07172 | histidinol-phosphate transaminase | Saccharomyces | Native |
| | | | | cerevisiae | |
| ScHISMN_59 | 59139.1 | P38635 | histidinol-phosphatase | Saccharomyces | Native |
| | | | | cerevisiae | |
| ScHISMN_60 | 65506.7 | P06633 | Imidazoleglycerol- | Saccharomyces | Native |
| | | | phosphate dehydratase | cerevisiae | |

Third-Round Genetic Engineering Results in Saccharomyces cerevisiae

Histamine production was further pursued in S. cerevisiae, and we designed plasmids to integrate additional copies of upstream pathway genes expressed by a strong constitutive promoter to avoid native regulation of a gene (Table 3). An expanded search was undertaken to test additional histidine decarboxylases that have similar sequences to the enzymes initially identified as active (Table 3).

In parallel we pursued modulating native gene expression to further improve histamine production. Our engineering approach was to take a best S. cerevisiae strain from the second round and test either a strong or weak constitutive promoter in place of the native promoter. Gene targets for promoter changes were selected to redirect flux supply precursors to histidine. Strain designs being tested include designs for increasing pentose phosphate pathway flux by expressing a non-native feedback deregulated glucose-6-phosphate dehydrogenase (zwf) and decreasing the “lower” pentose phosphate pathway flux thru the sugar isomerase enzymes.

Promoter replacements for lower expression of genes that are thought to be essential (i.e., cannot be deleted), but were expected to increase the upper glycolysis metabolite pool available for histamine production, targeted: 1) enolase (Eno2), to reduce flux through lower glycolysis, 2) pyruvate dehydrogenase (PDH, Lpd1) for lower flux through the C3/C2 node, and 3) pentose phosphate pathway sugar isomerases, which use the histamine metabolite precursor ribose-5-phosphate (Tall). An illustrative list of promoter-swap (“proswap”) and deletion (“knockout”) targets in S. cerevisiae includes:

| Annotation_name | Type | Promoter_name | Gene_name |
|---|---|---|---|
| YDR380W | knockout | | Aro10 |
| YDL047W | knockout | | Sit4 |
| YML035C | knockout | | Amd1 |
| YMR020W | knockout | | Fms1 |
| YNL229C | knockout | | Ure2 |
| YJL052W | proswap | pRnr1 | Tdh1 |
| YJR009C | proswap | pRnr1 | Tdh2 |
| YGR192C | proswap | pRnr1 | Tdh3 |
| YFL018C | proswap | pRnr1 | Lpd1 |
| YHR174W | proswap | pRnr1 | Eno2 |
| YNR001C | proswap | pRnr1 | Cit1 |
| YCR012W | proswap | pRnr1 | Pgk1 |
| YLR354C | proswap | pRnr1 | Tal1 |
| YBR117C | proswap | pRnr1 | Tkl2 |
| YPR074C | proswap | pRnr1 | Tkl1 |
| YML035C | proswap | pRev1 | Amd1 |
| YHR216W | proswap | pRev1 | Imd2 |
| YOR155C | proswap | pRev1 | Isn1 |
| YNL229C | proswap | pRev1 | Ure2 |
| YER086W | proswap | pRnr1 | Ilv1 |
| YDR380W | proswap | pRnr1 | Aro10 |
| YEL009C | proswap | pRev1 | Gcn4 |

Promoters were selected based on expression data from Lee et al [7].

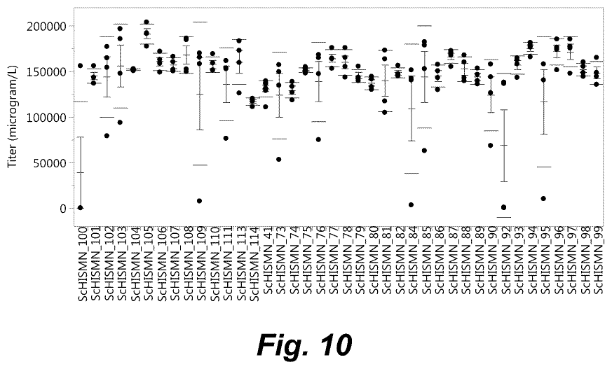

Additional genetic engineering results for S. cerevisiae are shown in Table 3 and FIG. 10. The parent strain for the strain designs shown in Table 3 (also the reference strain, ScHISMN_41) contained a histidine decarboxylase (UniProt ID J6KM89) and an ATP phosphoribosyltransferase (UniProt ID Q9Z472) harboring the amino acid substitutions N215K, L231F and T235A, and the ATP phosphoribosyltransferase from S. cerevisiae. The reference strain had a histamine titer of 131 mg/L.

Improved titer was observed in strains that expressed each of the following enzymes from a strong constitutive promoter:
1. Transketolase (EC 2.2.1.1) (SEQ ID NO: 27), which catalyzes the interconversion of sugars in the pentose phosphate pathway and produces ribose-5-phosphate, which is a precursor to PPRP, the initial metabolite in the histidine biosynthesis pathway.
2. Ribose-phosphate pyrophosphokinase (EC 2.7.6.1) (SEQ ID NO: 28) (highest titer: 191 mg/L relative to control in experiment 131 mg/L).
3. ATP phosphoribosyltransferase (EC 2.4.2.17) (SEQ ID NO: 3).
4. Trifunctional histidinol dehydrogenase (EC 1.1.1.23)/phosphoribosyl-AMP cyclohydrolase (EC 3.5.4.19)/phosphoribosyl-ATP diphosphatase (EC 3.6.1.31) (SEQ ID NO: 29).
5. Histidinol-phosphate aminotransferase (EC 2.6.1.9) (SEQ ID NO: 14).
6. 5′ProFAR isomerase (EC 5.3.1.16) (SEQ ID NO: 31).
7. Imidazole glycerol phosphate synthase (EC 4.3.1.B2) (SEQ ID NO: 21).
8. Triose-phosphate isomerase (EC 5.3.1.1), harboring the amino acid substitutions harboring the amino acid substitutions 1170V (SEQ ID NO: 32) or 1170T [8].
9. Glucose-6-phosphate 1-dehydrogenase (EC 1.1.1.49), harboring the amino acid substitution A243T (SEQ ID NO: 26).
10. Various histidine decarboxylases (EC 4.1.1.22):
   a. UniProt ID A0A089YPE5 (SEQ ID NO: 33)
   b. UniProt ID A0A126S6G9 (SEQ ID NO: 34)
   c. UniProt ID A0A0A1R6V3 (SEQ ID NO: 35)
   d. UniProt ID A0A1W0CM88 (SEQ ID NO: 36)
   e. UniProt ID P00862 (SEQ ID NO: 4)
   f. UniProt ID A0A0K6GJ74 (SEQ ID NO: 37)
   g. UniProt ID T0QL99 (SEQ ID NO: 38)
   h. UniProt ID A0A1B8HLR1 (SEQ ID NO: 39)

TABLE 3
Third-round genetic engineering results in Saccharomyces cerevisiae
Built and tested strain designs:

| | | | Enzyme | E1 | Enzyme | | Enzyme | Enzyme | | Enzyme | E3 | Enzyme |
|---|---|---|---|---|---|---|---|---|---|---|---|---|
| | | E1 | 1- | Modi- | 1- | E2 | 2- | 2- | E3 | 3- | Modi- | 3- |
| Strain | Titer | Uniprot | activity | fica- | source | Uniprot | activity | source | Uniprot | activity | fica- | source |
| name | (μg/L) | ID | name | tions | organism | ID | name | organism | ID | name | tions | organism |
| Sc- | 39059 | A0A0C- | Histidine | | Lactobacillus | | | | | | | |
| HISM- | | 1PR48 | decarboxy- | | fructivorans | | | | | | | |
| N_100 | | | lase | | | | | | | | | |
| Sc- | 144871 | T0QL99 | Histidine | | Aeromonas | | | | | | | |
| HISM- | | | decarboxy- | | salmonicida | | | | | | | |
| N_101 | | | lase | | subsp. | | | | | | | |
| | | | | | pectinolytica | | | | | | | |
| | | | | | 34mel | | | | | | | |
| Sc- | 143763 | A0A1B8- | Histidine | | Morganella | | | | | | | |
| HISM- | | HLR1 | decarboxy- | | psychro- | | | | | | | |
| N_102 | | | lase | | tolerans | | | | | | | |
| Sc- | 155931 | Q9Z472 | ATP | | Coryne- | P00815 | tri- | Saccharo- | P0A717 | Ribose- | | Esche- |
| HISM- | | | phosphori- | | bacterium | | functional | myces | | phos- | | richia |
| N_103 | | | bosyl- | | glutamicum | | histidinol | cerevisiae | | phate | | coli |
| | | | transferase | | (strain | | dehydro- | S288c | | pyro- | | (strain |
| | | | | | ATCC | | genase/ | | | phos- | | K12) |
| | | | | | 13032/ | | phos- | | | pho- | | |
| | | | | | DSM | | phoribo- | | | kinase | | |
| | | | | | 20300/ | | syl-AMP | | | | | |
| | | | | | JCM | | cyclo- | | | | | |
| | | | | | 1318/ | | hydrolase/ | | | | | |
| | | | | | LMG | | phos- | | | | | |
| | | | | | 3730/ | | phoribo- | | | | | |
| | | | | | NCIMB | | syl-ATP | | | | | |
| | | | | | 10025) | | diphos- | | | | | |
| | | | | | | | phatase | | | | | |
| Sc- | 151846 | P0A717 | Ribose- | | Escherichia | | | | | | | |
| HISM- | | | phosphate | | coli | | | | | | | |
| N_104 | | | pyrophos- | | (strain | | | | | | | |
| | | | pho- | | K12) | | | | | | | |
| | | | kinase | | | | | | | | | |
| Sc- | 191110 | Q12265 | Ribose- | | Saccharo- | P38620 | Ribose- | Saccharo- | Q680A5 | Ribose- | | Arabi- |
| HISM- | | | phosphate | | myces | | phosphate | myces | | phos- | | dopsis |
| N_105 | | | pyrophos- | | cerevisiae | | pyrophos- | cerevisiae | | phate | | thaliana |
| | | | pho- | | (strain | | phokinase | S288c | | pyro- | | (Mouse- |
| | | | kinase | | ATCC | | | | | phos- | | ear |
| | | | | | 204508/ | | | | | pho- | | cress) |
| | | | | | S288c) | | | | | kinase | | |
| | | | | | (Baker's | | | | | | | |
| | | | | | yeast) | | | | | | | |
| Sc- | 160586 | P32895 | Ribose- | | Saccharo- | P38689 | Ribose- | Saccharo- | | | | |
| HISM- | | | phosphate | | myces | | phosphate | myces | | | | |
| N_106 | | | pyrophos- | | cerevisiae | | pyrophos- | cerevisiae | | | | |
| | | | pho- | | (strain | | phokinase | S288c | | | | |
| | | | kinase | | ATCC | | | | | | | |
| | | | | | 204508/ | | | | | | | |
| | | | | | S288c) | | | | | | | |
| | | | | | (Baker's | | | | | | | |
| | | | | | yeast) | | | | | | | |
| Sc- | 157191 | P23254 | Trans- | | Saccharo- | P32895 | Ribose- | Saccharo- | P38689 | Ribose- | | Saccharo- |
| HISM- | | | ketolase | | myces | | phosphate | myces | | phos- | | myces |
| N_107 | | | | | cerevisiae | | pyrophos- | cerevisiae | | phate | | cerevisiae |
| | | | | | (strain | | phokinase | S288c | | pyro- | | S288c |
| | | | | | ATCC | | | | | phos- | | |
| | | | | | 204508/ | | | | | pho- | | |
| | | | | | S288c) | | | | | kinase | | |
| | | | | | (Baker's | | | | | | | |
| | | | | | yeast) | | | | | | | |
| Sc- | 168183 | P23254 | Trans- | | Saccharo- | Q12265 | Ribose- | Saccharo- | P38620 | Ribose- | | Saccharo- |
| HISM- | | | ketolase | | myces | | phosphate | myces | | phos- | | myces |
| N_108 | | | | | cerevisiae | | pyrophos- | cerevisiae | | phate | | cerevisiae |
| | | | | | (strain | | phokinase | S288c | | pyro- | | S288c |
| | | | | | ATCC | | | | | phos- | | |
| | | | | | 204508/ | | | | | pho- | | |
| | | | | | S288c) | | | | | kinase | | |
| | | | | | (Baker's | | | | | | | |
| | | | | | yeast) | | | | | | | |
| Sc- | 125249 | P23254 | Trans- | | Saccharo- | Q12265 | Ribose- | Saccharo- | Q680A5 | Ribose- | | Arabi- |
| HISM- | | | ketolase | | myces | | phosphate | myces | | phos- | | dopsis |
| N_109 | | | | | cerevisiae | | pyrophos- | cerevisiae | | phate | | thaliana |
| | | | | | (strain | | phokinase | S288c | | pyro- | | (Mouse- |
| | | | | | ATCC | | | | | phos- | | ear |
| | | | | | 204508/ | | | | | pho- | | cress) |
| | | | | | S288c) | | | | | kinase | | |
| | | | | | (Baker's | | | | | | | |
| | | | | | yeast) | | | | | | | |
| Sc- | 157653 | P23254 | Trans- | | Saccharo- | P0A717 | Ribose- | Esche- | | | | |
| HISM- | | | ketolase | | myces | | phosphate | richia | | | | |
| N_110 | | | | | cerevisiae | | pyrophos- | coli | | | | |
| | | | | | (strain | | phokinase | (strain | | | | |
| | | | | | ATCC | | | K12) | | | | |
| | | | | | 204508/ | | | | | | | |
| | | | | | S288c) | | | | | | | |
| | | | | | (Baker's | | | | | | | |
| | | | | | yeast) | | | | | | | |
| Sc- | 136093 | P23254 | Trans- | | Saccharo- | P15019 | Trans- | Saccharo- | P0A717 | Ribose- | | Esche- |
| HISM- | | | ketolase | | myces | | aldolase | myces | | phos- | | richia |
| N_111 | | | | | cerevisiae | | | cerevisiae | | phate | | coli |
| | | | | | (strain | | | S288c | | pyro- | | (strain |
| | | | | | ATCC | | | | | phos- | | K12) |
| | | | | | 204508/ | | | | | pho- | | |
| | | | | | S288c) | | | | | kinase | | |
| | | | | | (Baker's | | | | | | | |
| | | | | | yeast) | | | | | | | |
| Sc- | | P06775 | Histidine | | Saccharo- | | | | | | | |
| HISM- | | | permease | | myces | | | | | | | |
| N_112 | | | | | cerevisiae | | | | | | | |
| | | | | | (strain | | | | | | | |
| | | | | | ATCC | | | | | | | |
| | | | | | 204508/ | | | | | | | |
| | | | | | S288c) | | | | | | | |
| | | | | | (Baker's | | | | | | | |
| | | | | | yeast) | | | | | | | |
| Sc- | 160417 | P00815 | trifunctional | | Saccharo- | P40545 | 5'ProFAR | Saccharo- | O59667 | Bifunc- | | Schizo- |
| HISM- | | | histidinol | | myces | | isomerase | myces | | tional | | saccharo- |
| N_113 | | | dehydro- | | cerevisiae | | | cerevisiae | | phos- | | myces |
| | | | genase/ | | (strain | | | S288c | | phoribo- | | pombe |
| | | | phos- | | ATCC | | | | | syl-AMP | | ATCC |
| | | | phoribo- | | 204508/ | | | | | cyclo- | | 24843 |
| | | | syl-AMP | | S288c) | | | | | hydro- | | |
| | | | cyclo- | | (Baker's | | | | | lase and | | |
| | | | hydro- | | yeast) | | | | | phos- | | |
| | | | lase/ | | | | | | | phoribo- | | |
| | | | phos- | | | | | | | syl-ATP | | |
| | | | phoribo- | | | | | | | pyro- | | |
| | | | syl-ATP | | | | | | | phos- | | |
| | | | diphos- | | | | | | | phatase | | |
| | | | phatase | | | | | | | | | |
| Sc- | 116907 | P06633 | Imidazole- | | Saccharo- | P07172 | Histidinol- | Saccharo- | P38635 | Histidi- | | Saccharo- |
| HISM- | | | glycerol- | | myces | | phosphate | myces | | nol- | | myces |
| N_114 | | | phosphate | | cerevisiae | | amino- | cerevisiae | | phos- | | cerevisiae |
| | | | dehydratase | | (strain | | trans- | S288c | | phatase | | S288c |
| | | | | | ATCC | | ferase | | | | | |
| | | | | | 204508/ | | | | | | | |
| | | | | | S288c) | | | | | | | |
| | | | | | (Baker's | | | | | | | |
| | | | | | yeast) | | | | | | | |
| Sc- | 131308 | | | | | | | | | | | |
| HISM- | | | | | | | | | | | | |
| N_41 | | | | | | | | | | | | |
| Sc- | 123614 | P00815 | trifunctional | | Saccharo- | | | | | | | |
| HISM- | | | histidinol | | myces | | | | | | | |
| N_73 | | | dehydro- | | cerevisiae | | | | | | | |
| | | | genase/ | | (strain | | | | | | | |
| | | | phos- | | ATCC | | | | | | | |
| | | | phoribo- | | 204508/ | | | | | | | |
| | | | syl-AMP | | S288c) | | | | | | | |
| | | | cyclohydro- | | (Baker's | | | | | | | |
| | | | lase/ | | yeast) | | | | | | | |
| | | | phos- | | | | | | | | | |
| | | | phoribo- | | | | | | | | | |
| | | | syl-ATP | | | | | | | | | |
| | | | diphos- | | | | | | | | | |
| | | | phatase | | | | | | | | | |
| Sc- | 129393 | P06633 | Imidazole- | | Saccharo- | | | | | | | |
| HISM- | | | glycerol- | | myces | | | | | | | |
| N_74 | | | phosphate | | cerevisiae | | | | | | | |
| | | | dehydratase | | (strain | | | | | | | |
| | | | | | ATCC | | | | | | | |
| | | | | | 204508/ | | | | | | | |
| | | | | | S288c) | | | | | | | |
| | | | | | (Baker's | | | | | | | |
| | | | | | yeast) | | | | | | | |
| Sc- | 151455 | P07172 | Histidinol- | | Saccharo- | | | | | | | |
| HISM- | | | phosphate | | myces | | | | | | | |
| N_75 | | | aminotrans- | | cerevisiae | | | | | | | |
| | | | ferase | | (strain | | | | | | | |
| | | | | | ATCC | | | | | | | |
| | | | | | 204508/ | | | | | | | |
| | | | | | S288c) | | | | | | | |
| | | | | | (Baker's | | | | | | | |
| | | | | | yeast) | | | | | | | |
| Sc- | 138833 | P00498 | ATP | | Saccharo- | | | | | | | |
| HISM- | | | phosphori- | | myces | | | | | | | |
| N_76 | | | bosyl- | | cerevisiae | | | | | | | |
| | | | transferase | | (strain | | | | | | | |
| | | | | | ATCC | | | | | | | |
| | | | | | 204508/ | | | | | | | |
| | | | | | S288c) | | | | | | | |
| | | | | | (Baker's | | | | | | | |
| | | | | | yeast) | | | | | | | |
| Sc- | 164217 | P40545 | 5′ProFAR | | Saccharo- | | | | | | | |
| HISM- | | | isomerase | | myces | | | | | | | |
| N_77 | | | | | cerevisiae | | | | | | | |
| | | | | | (strain | | | | | | | |
| | | | | | ATCC | | | | | | | |
| | | | | | 204508/ | | | | | | | |
| | | | | | S288c) | | | | | | | |
| | | | | | (Baker's | | | | | | | |
| | | | | | yeast) | | | | | | | |
| Sc- | 159871 | P33734 | Imidazole | | Saccharo- | | | | | | | |
| HISM- | | | glycerol | | myces | | | | | | | |
| N_78 | | | phosphate | | cerevisiae | | | | | | | |
| | | | synthase | | (strain | | | | | | | |
| | | | | | ATCC | | | | | | | |
| | | | | | 204508/ | | | | | | | |
| | | | | | S288c) | | | | | | | |
| | | | | | (Baker's | | | | | | | |
| | | | | | yeast) | | | | | | | |
| Sc- | 145179 | P00942 | Triosephos- | I170V | Saccharo- | | | | | | | |
| HISM- | | | phate | | myces | | | | | | | |
| N_79 | | | isomerase | | cerevisiae | | | | | | | |
| | | | | | (strain | | | | | | | |
| | | | | | ATCC | | | | | | | |
| | | | | | 204508/ | | | | | | | |
| | | | | | S288c) | | | | | | | |
| | | | | | (Baker's | | | | | | | |
| | | | | | yeast) | | | | | | | |
| Sc- | 137192 | P00942 | Triosephos- | I170T | Saccharo- | | | | | | | |
| HISM- | | | phate | | myces | | | | | | | |
| N_80 | | | isomerase | | cerevisiae | | | | | | | |
| | | | | | (strain | | | | | | | |
| | | | | | ATCC | | | | | | | |
| | | | | | 204508/ | | | | | | | |
| | | | | | S288c) | | | | | | | |
| | | | | | (Baker's | | | | | | | |
| | | | | | yeast) | | | | | | | |
| Sc- | 139699 | Q9Z472 | ATP | | Coryne- | | | | | | | |
| HISM- | | | phosphori- | | bacterium | | | | | | | |
| N_81 | | | bosyl- | | glutamicum | | | | | | | |
| | | | transferase | | (strain | | | | | | | |
| | | | | | ATCC | | | | | | | |
| | | | | | 13032/ | | | | | | | |
| | | | | | DSM | | | | | | | |
| | | | | | 20300/ | | | | | | | |
| | | | | | JCM | | | | | | | |
| | | | | | 1318/ | | | | | | | |
| | | | | | LMG | | | | | | | |
| | | | | | 3730/ | | | | | | | |
| | | | | | NCIMB | | | | | | | |
| | | | | | 10025) | | | | | | | |
| Sc- | 148665 | Q9Z472 | ATP | N215K, | Coryne- | | | | | | | |
| HISM- | | | phosphori- | L231F, | bacterium | | | | | | | |
| N_82 | | | bosyl- | T235A | glutamicum | | | | | | | |
| | | | transferase | | (strain | | | | | | | |
| | | | | | ATCC | | | | | | | |
| | | | | | 13032/ | | | | | | | |
| | | | | | DSM | | | | | | | |
| | | | | | 20300/ | | | | | | | |
| | | | | | JCM | | | | | | | |
| | | | | | 1318/LMG | | | | | | | |
| | | | | | 3730/ | | | | | | | |
| | | | | | NCIMB | | | | | | | |
| | | | | | 10025) | | | | | | | |
| Sc- | 109350 | Q9Z472 | ATP | N215K, | Coryne- | P00815 | tri- | Saccharo- | | | | |
| HISM- | | | phosphori- | L231F, | bacterium | | functional | myces | | | | |
| N_84 | | | bosyl- | T235A | glutamicum | | histidinol | cerevisiae | | | | |
| | | | transferase | | (strain | | dehydro- | S288c | | | | |
| | | | | | ATCC | | genase/ | | | | | |
| | | | | | 13032/ | | phos- | | | | | |
| | | | | | DSM | | phoribo- | | | | | |
| | | | | | 20300/ | | syl-AMP | | | | | |
| | | | | | JCM | | cyclo- | | | | | |
| | | | | | 1318/ | | hydro- | | | | | |
| | | | | | LMG | | lase/ | | | | | |
| | | | | | 3730/ | | phos- | | | | | |
| | | | | | NCIMB | | phoribo- | | | | | |
| | | | | | 10025) | | syl-ATP | | | | | |
| | | | | | | | diphos- | | | | | |
| | | | | | | | phatase | | | | | |
| Sc- | 144154 | Q9Z472 | ATP | | Coryne- | P00815 | tri- | Saccharo- | P00942 | Triose- | I170V | Saccharo- |
| HISM- | | | phosphori- | | bacterium | | functional | myces | | phos- | | myces |
| N_85 | | | bosyl- | | glutamicum | | histidinol | cerevisiae | | phate | | cerevisiae |
| | | | transferase | | (strain | | dehydro- | S288c | | iso- | | S288c |
| | | | | | ATCC | | genase/ | | | merase | | |
| | | | | | 13032/ | | phos- | | | | | |
| | | | | | DSM | | phoribo- | | | | | |
| | | | | | 20300/ | | syl-AMP | | | | | |
| | | | | | JCM | | cyclo- | | | | | |
| | | | | | 1318/ | | hydro- | | | | | |
| | | | | | LMG | | lase/ | | | | | |
| | | | | | 3730/ | | phos- | | | | | |
| | | | | | NCIMB | | phoribo- | | | | | |
| | | | | | 10025) | | syl-ATP | | | | | |
| | | | | | | | diphos- | | | | | |
| | | | | | | | phatase | | | | | |
| Sc- | 145171 | Q9Z472 | ATP | N215K, | Coryne- | P00815 | tri- | Saccharo- | P00942 | Triose- | I170V | Saccharo- |
| HISM- | | | phosphori- | L231F, | bacterium | | functional | myces | | phos- | | myces |
| N_86 | | | bosyl- | T235A | glutamicum | | histidinol | cerevisiae | | phate | | cerevisiae |
| | | | transferase | | (strain | | dehydro- | S288c | | iso- | | S288c |
| | | | | | ATCC | | genase/ | | | merase | | |
| | | | | | 13032/ | | phos- | | | | | |
| | | | | | DSM | | phoribo- | | | | | |
| | | | | | 20300/ | | syl-AMP | | | | | |
| | | | | | JCM | | cyclo- | | | | | |
| | | | | | 1318/ | | hydrolase/ | | | | | |
| | | | | | LMG | | phos- | | | | | |
| | | | | | 3730/ | | phoribo- | | | | | |
| | | | | | NCIMB | | syl-ATP | | | | | |
| | | | | | 10025) | | diphos- | | | | | |
| | | | | | | | phatase | | | | | |
| Sc- | 166497 | Q9Z472 | ATP | | Coryne- | P00815 | tri- | Saccharo- | A4QEF2 | Glucose- | A243T | Coryne- |
| HISM- | | | phosphori- | | bacterium | | functional | myces | | 6-phos- | | bacterium |
| N_87 | | | bosyl- | | glutamicum | | histidinol | cerevisiae | | phate | | gluta- |
| | | | transferase | | (strain | | dehydro- | S288c | | 1- | | micum |
| | | | | | ATCC | | genase/ | | | dehydro- | | (strain R) |
| | | | | | 13032/ | | phos- | | | genase | | |
| | | | | | DSM | | phoribo- | | | | | |
| | | | | | 20300/ | | syl-AMP | | | | | |
| | | | | | JCM | | cyclo- | | | | | |
| | | | | | 1318/ | | hydro- | | | | | |
| | | | | | LMG | | lase/ | | | | | |
| | | | | | 3730/ | | phos- | | | | | |
| | | | | | NCIMB | | phoribo- | | | | | |
| | | | | | 10025) | | syl-ATP | | | | | |
| | | | | | | | diphos- | | | | | |
| | | | | | | | phatase | | | | | |
| Sc- | 152555 | Q9Z472 | ATP | N215K, | Coryne- | P00815 | tri- | Saccharo- | A4QEF2 | Glucose- | A243T | Coryne- |
| HISM- | | | phosphori- | L231F, | bacterium | | functional | myces | | 6-phos- | | bacterium |
| N_88 | | | bosyl- | T235A | glutamicum | | histidinol | cerevisiae | | phate | | gluta- |
| | | | transferase | | (strain | | dehydro- | S288c | | 1- | | micum |
| | | | | | ATCC | | genase/ | | | dehydro- | | (strain R) |
| | | | | | 13032/ | | phos- | | | genase | | |
| | | | | | DSM | | phoribo- | | | | | |
| | | | | | 20300/ | | syl-AMP | | | | | |
| | | | | | JCM | | cyclo- | | | | | |
| | | | | | 1318/ | | hydro- | | | | | |
| | | | | | LMG | | lase/ | | | | | |
| | | | | | 3730/ | | phos- | | | | | |
| | | | | | NCIMB | | phoribo- | | | | | |
| | | | | | 10025) | | syl-ATP | | | | | |
| | | | | | | | diphos- | | | | | |
| | | | | | | | phatase | | | | | |
| Sc- | 143866 | O66000 | Histidine | | Oenococcus | | | | | | | |
| HISM- | | | decarboxy- | | oeni | | | | | | | |
| N_89 | | | lase | | (Leuconostoc | | | | | | | |
| | | | | | oenos) | | | | | | | |
| Sc- | 124157 | A0A0R- | Histidine | | Lactobacillus | | | | | | | |
| HISM- | | 1Y874 | decarboxy- | | aviarius | | | | | | | |
| N_90 | | | lase | | subsp. | | | | | | | |
| | | | | | aviarius | | | | | | | |
| | | | | | DSM | | | | | | | |
| | | | | | 20655 | | | | | | | |
| Sc- | 68849 | A0A1H- | Histidine | S9R | Pseudo- | | | | | | | |
| HISM- | | 1TEB8 | decarboxy- | | monas | | | | | | | |
| N_92 | | | lase | | sp. bs2935 | | | | | | | |
| Sc- | 157127 | A0A089- | Histidine | | Pseudo- | | | | | | | |
| HISM- | | YPE5 | decarboxy- | | monas | | | | | | | |
| N_93 | | | lase | | rhizosphaerae | | | | | | | |
| Sc- | 175497 | A0A126- | Histidine | | Pseudo- | | | | | | | |
| HISM- | | S6G9 | decarboxy- | | monas putida | | | | | | | |
| N_94 | | | lase | | (Arthrobacter | | | | | | | |
| | | | | | siderocap- | | | | | | | |
| | | | | | sulatus) | | | | | | | |
| Sc- | 116642 | A0A0J6- | Histidine | | Chromo- | | | | | | | |
| HISM- | | KM89 | decarboxy- | | bacterium sp. | | | | | | | |
| N_95 | | | lase | | LK1 | | | | | | | |
| Sc- | 171681 | A0A0A1- | Histidine | | Citrobacter | | | | | | | |
| HISM- | | R6V3 | decarboxy- | | pasteurii | | | | | | | |
| N_96 | | | lase | | | | | | | | | |
| Sc- | 171393 | A0A1W- | Histidine | | Chromo- | | | | | | | |
| HISM- | | 0CM88 | decarboxy- | | bacterium | | | | | | | |
| N_97 | | | lase | | haemolyticum | | | | | | | |
| Sc- | 152065 | P00862 | Histidine | | Lactobacillus | | | | | | | |
| HISM- | | | decarboxy- | | sp. (strain | | | | | | | |
| N_98 | | | lase | | 30a) | | | | | | | |
| Sc- | 148362 | A0A0K6- | Histidine | | Lactobacillus | | | | | | | |
| HISM- | | GJ74 | decarboxy- | | reuteri | | | | | | | |
| N_99 | | | lase | | | | | | | | | |

Note:
E1, E2, and E3 genes were codon-optimized according to modified codon usage for Cg and Sc

### Example 2—Host Evaluation for Histamine Production

Histamine production was also tested in two additional hosts, Bacillus subtilus and Yarrowia lipolytica, which were engineered to express the enzymes from the best-performing Corynebacterium glutamicum and Saccharomyces cerevisiae strains.

Host evaluation designs were selected to express 1-3 enzymes and, each design was tested with four different codon optimizations based on the host organisms C. glutamicum, S. cerevisiae, B. subtilis, and Y. lipolytica. The codon optimizations tested were based on the Kazusa codon usage tables tabulated for each host for gene codon optimization (www.kazusa.or.jp/codon/).

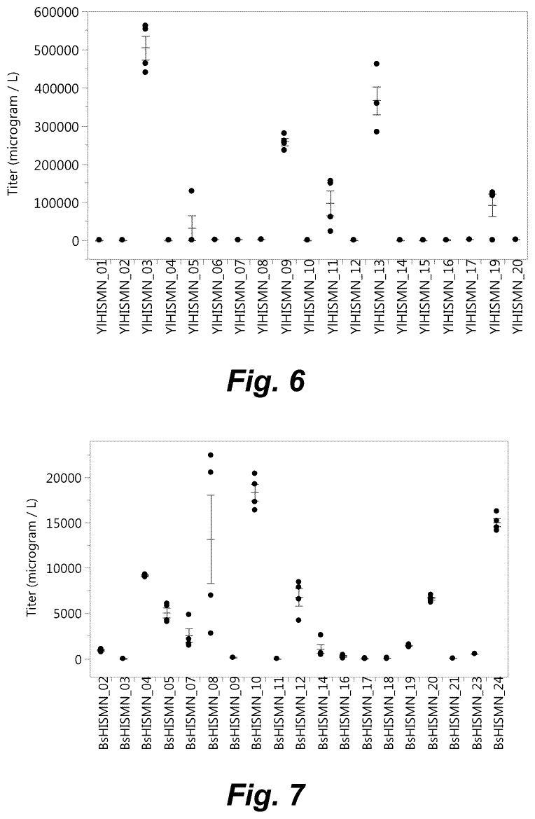

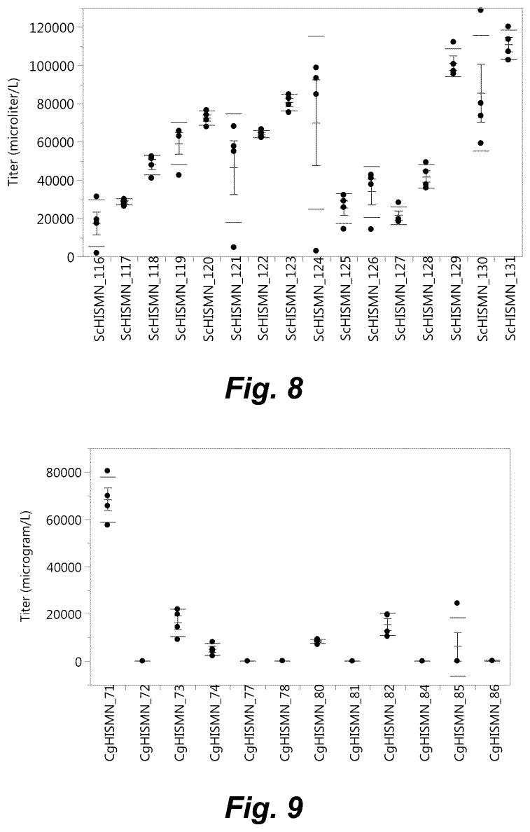

Histamine production was demonstrated in Y. lipolytica (FIG. 6) and B. subtilis (FIG. 7) and further improved in C. glutamicum (FIG. 9) and S. cerevisiae (FIG. 8).

In Y. lipolytica (FIG. 6, Table 4, below) the best performing strain produced 505 mg/L histamine and expressed the histidine decarboxylase from Acinetobacter baumannii strain AB0057 (UniProt ID B7I459), where the DNA sequence was codon-optimized for Y. lipolytica, and the ATP phosphoribosyltransferase from S. cerevisiae S288c (UniProt ID P00498), where the DNA sequence was codon optimized for Y. lipolytica. The same two genes were also tested where the DNA sequence was codon-optimized for B. subtilis and S. cerevisiae and the resulting strains produced no histamine titer.

The second best-performing strain in Y. lipolytica also expressed the histidine decarboxylase from Acinetobacter baumannii strain AB0057 (UniProt ID B71459), where the DNA sequence was codon-optimized for Y. lipolytica, and the ATP phosphoribosyltransferase from Salmonella typhimurium LT2 (UniProt ID P00499), where the DNA was codon optimized for Y. lipolytica. Versions of these two genes were also tested where the DNA sequence was codon optimized for B. subtilis (which produced 0 titer), codon-optimized for S. cerevisiae (which produced 33 micrograms histamine) and codon-optimized using a combined codon table for S. cerevisiae and C. glutamicum (produced 97 mg/L histamine).

The third best-performing strain in Y. lipolytica produced 258 mg/L histamine and expressed the histidine decarboxylase from Chromobacterium sp. LK1 (UniProt ID A0A0J6KM89), where the DNA sequence was codon optimized for Y. lipolytica, and the ATP phosphoribosyltransferase from C. glutamicum ATCC 13032 (UniProt ID Q9Z472) harboring the amino acid substitutions N215K, L23IF, T235A (SEQ ID NO: 7), where the DNA sequence was codon-optimized for Y. lipolytica (SEQ ID NO: 64). Versions of these two genes were also tested where the DNA sequences were codon-optimized for S. cerevisiae (SEQ ID NO: 65, 66) or B. subtilis (SEQ ID NO: 67, 68), and these Y. lipolytica strains produced 1.8 mg/L and 0.3 mg/L, respectively. Accordingly, codon-optimization of genes affects expression in Y. lipolytica.

In B. subtilis (FIG. 7, Table 5, below) the best performing strain produced 18 mg/L histamine and expressed the histamine decarboxylase from Lactobacillus sp. (strain 30a) (UniProt ID P00862)(SEQ ID NO: 4) with the ATP phosphoribosyltransferase from Salmonella typhimurium LT2 (UniProt ID P00499)(SEQ ID NO: 5) where the DNA sequence was codon optimized for Bacillus subtilis (SEQ ID NO: 69, 59). The same two genes were also tested where the DNA sequence was codon-optimized for S. cerevisiae (SEQ ID NO: 70, 60) or modified codon usage table for C. glutamicum and S. cerevisiae (SEQ ID NO: 71, 62), and these strains produced 6.7 mg/L or 0 mg/L histamine, respectively.

The host evaluation designs were also tested in S. cerevisiae and C. glutamicum. In S. cerevisiae (FIG. 8, Table 6, below) the best-performing strain produced 111 mg/L histamine and expressed the histamine decarboxylase from Chromobacterium sp. LK1 (UniProt ID A0A0J6KM89)(SEQ ID NO: 51) and the ATP phosphoribosyltransferase from Saccharomyces cerevisiae S288c (UniProt ID P00498)(SEQ ID NO: 3), where the DNA sequences were codon-optimized for Y. lipolytica (SEQ ID NO: 63, 53). The same two genes were also tested where the DNA sequences were codon optimized for S. cerevisiae (SEQ ID NO: 65, 57) and B. subtilis (SEQ ID NO: 67, 55) produced 86 mg/L and 101 mg/L, respectively.

In C. glutamicum (FIG. 9, Table 7), the best-performing strain produced 68 mg/L histamine and expressed the histamine decarboxylase from Acinetobacter baumannii (strain AB0057) (UniProt ID B7I459) (SEQ ID NO: 1) with the ATP phosphoribosyltransferase from Saccharomyces cerevisiae S288c (UniProt ID P00498) (SEQ ID NO: 3) where the DNA sequences were codon-optimized using a modified codon usage table for C. glutamicum and S. cerevisiae (SEQ ID NO: 72, 73). The same two genes were also tested where the DNA sequence was codon-optimized for Y. lipolytica (SEQ ID NO: 52, 53) or S. cerevisiae (SEQ ID NO: 56, 57), and these strains produced 16 mg/L and 18 microgram/L histamine, respectively.

The second best-performing strain in C. glutamicum produced 15 mg/L histamine and also expressed a histidine decarboxylase from Acinetobacter baumannii strain AB0057 (UniProt ID B7I459) (SEQ ID NO: 1), where the DNA sequence was codon optimized for Y. lipolytica (SEQ ID NO: 52), and an ATP phosphoribosyltransferase from Salmonella typhimurium LT2 (UniProt ID P00499) (SEQ ID NO: 5), where the DNA was codon optimized for Y. lipolytica (SEQ ID NO: 58). These same two genes were also tested, where the DNA sequences were codon-optimized for B. subtilis (SEQ ID NO: 54, 59) (which produced 8 mg/L histamine) or codon-optimized for S. cerevisiae (SEQ ID NO: 56, 60)(which produced 9.3 mg/L histamine).

Since the best performing strain is in the host Y. lipolytica, further strain improvements can be pursued in this host organism. Designs that can further enhance histamine production in Y. lipolytica include:
1. Transketolase (EC 2.2.1.1) (SEQ ID NO: 27), which catalyzes the interconversion of sugars in the pentose phosphate pathway and produces ribose-5-phosphate, which is a precursor to PPRP, the initial metabolite in the histidine biosynthesis pathway.
2. Ribose-phosphate pyrophosphokinase (EC 2.7.6.1) (SEQ ID NO: 28).
3. ATP phosphoribosyltransferase (EC 2.4.2.17) (SEQ ID NO: 5).
4. Trifunctional histidinol dehydrogenase (EC 1.1.1.23)/phosphoribosyl-AMP cyclohydrolase (EC 3.5.4.19)/phosphoribosyl-ATP diphosphatase (EC 3.6.1.31) (SEQ ID NO: 20).
5. Histidinol-phosphate aminotransferase (EC 2.6.1.9) (SEQ ID NO: 14).
6. 5′ProFAR isomerase (EC 5.3.1.16) (SEQ ID NO: 31).
7. Imidazole glycerol phosphate synthase (EC 4.3.1.B2) (SEQ ID NO: 21).
8. Triose-phosphate isomerase (EC 5.3.1.1) harboring the amino acid substitution I170V (SEQ ID NO: 32).
9. Glucose-6-phosphate 1-dehydrogenase (EC 1.1.1.49) harboring the amino acid substitution A243T (SEQ ID NO: 26).
10. Various histidine decarboxylases:
   a. UniProt ID A0A089YPE5 (SEQ ID NO: 33)
   b. UniProt ID A0A126S6G9 (SEQ ID NO: 34)
   c. UniProt ID A0A0A1R6V3 (SEQ ID NO: 35)
   d. UniProt ID A0A1W0CM88 (SEQ ID NO: 36)
   e. UniProt ID P00862 (SEQ ID NO: 4)
   f. UniProt ID A0A0K6GJ74 (SEQ ID NO: 37)
   g. UniProt ID T0QL99 (SEQ ID NO: 38)
   h. UniProt ID A0A1B8HLR1 (SEQ ID NO: 39)

### Example 3—Improvement of Histamine Production in Yarrowia lipolytica Engineered to Produce Histamine

Three improvement rounds of genetic engineering were carried out in Yarrowia lipolytica.

First-Improvement Round Genetic Engineering in Yarrowia lipolytica

Strategy: Improve flux into histidine and then histamine by overexpression of two enzymes.

| | UniProt | | | Codon | |
|---|---|---|---|---|---|
| Enzyme Name | ID | Organism | Description | optmization | Mutation |
| Histidine | B71459 | Acinetobacter | Last decarboxylation | Yarrowia | None |
| decarboxylase | | baumannii | step of histamine | lypolytica | |
| (HDC) | | | biosynthesis | | |
| ATP | P00498 | Saccharomyces | Upstream step of | Yarrowia | None |
| phosphoribosyltransferase | | cerevisiae | histidine biosynthesis. | lypolytica | |
| (ATP-PRase) | | | Utilization of ATP | | |
| | | | to covert PRPP to | | |
| | | | PR-ATP | | |

Summary: ATP phosphoribosyltranslerase catalyzes the first committed step of histidine biosynthesis pathway. This enzyme would be allosterically feedback-inhibited by histidine and competitively inhibited by AMP and ADP. The results did not indicate activity and/or inhibition of P00498.

Second-Improvement Round Genetic Engineering in Yarrowia lipolytica

Strategy: Overexpression of one enzyme. The final step of histamine biosynthesis was enhanced by utilizing the best first-round histidine decarboxylase which was modified to include a solubility tag to improve protein folding.

| | UniProt | | | Codon | |
|---|---|---|---|---|---|
| Enzyme Name | ID | Organism | Description | optmization | Mutation |
| Histidine | B71459 | Acinetobacter | Last decarboxylation | Yarrowia | None |
| decarboxylase | | baumannii | step of histamine | lypolytica | |
| (HDC) | | | biosynthesis | | |

Summary: The histidine decarboxylase used for the second round of genetic engineering was the same as for the first round, although the codon optimization was different. Furthermore, an N-terminal solubility tag (MQYKLALNGKTLKGETTTEAVDAATAEKVFKQYANDNGVDGEWTYDDATKTFT VT, SEQ ID NO:142) was included in the second-round enzyme.

Third-Improvement Round Genetic Engineering in Yarrowia lipolytica

Strategy: Overexpression of two enzymes in pathways upstream of histidine biosynthesis to improve flux into phosphoribosyl pyrophosphate (PRPP).

| | UniProt | | | Codon | |
|---|---|---|---|---|---|
| Enzyme Name | ID | Organism | Description | optmization | Mutation |
| Ribose-phosphate | E7EAU9 | Bacillus | ATP dependent step for | Yarrowia | L135I |
| pyrophosphokinase | | amyloliquefaciens | synthesis of PRPP | lypolytica | |
| (RPPK) | | | | | |
| Glocose-6-phosphate | A4QEF2 | Corynebacterium | Upstream pathway to | Yarrowia | A243T |
| 1-dehydrogenase | | glutamicum | push carbon flux into | lypolytica | |
| (G6PDH) | | | ribose-5-phosphate | | |

Summary: Ribose-phosphate pyrophosphokinase is competitively inhibited ADP. The L135I mutation at the ATP binding site on the enzyme relieves ADP inhibition. This strain expressed histamine at a titer of 1.68 g/L of culture medium.

TABLE 4
First-round results for histamine production in Yarrowia lipolytica

| | | | | | | | | | | |
|---|---|---|---|---|---|---|---|---|---|---|
| | | E1 | Enzyme 1- | Enzyme 1- | E1 Codon | E2 | Enzyme 2- | E2 | Enzyme 2- | E2 Codon |
| Strain | Titer | Uniprot | activity | source | Optimization | Uniprot | activity | Modifi- | source | Optimization |
| name | (μg/L) | ID | name | organism | Abbrev. | ID | name | cations | organism | Abbrev. |

Yarrowia lipolytica

| | | | | | | | | | | |
|---|---|---|---|---|---|---|---|---|---|---|
| YIHISMN_ | 0 | B71459 | Histidine | Acinetobacter | Bacillus | P00498 | ATP | | Saccharomyces | Bacillus |
| 01 | | | decarboxylase | baumannii | subtilis | | phosphoribosyl- | | cerevisiae | subtilis |
| | | | | (strain | | | transferase | | S288c | |
| | | | | AB0057) | | | | | | |
| YIHISMN_ | 0 | B71459 | Histidine | Acinetobacter | Saccharo- | P00498 | ATP | | Saccharomyces | Saccharo- |
| 02 | | | decarboxylase | baumannii | myces | | phosphoribosyl- | | cerevisiae | myces |
| | | | | (strain | cerevisiae | | transferase | | S288c | cerevisiae |
| | | | | AB0057) | | | | | | |
| YIHISMN_ | 505019 | B71459 | Histidine | Acinetobacter | Yarrowia | P00498 | ATP | | Saccharomyces | Yarrowia |
| 03 | | | decarboxylase | baumannii | lipolytica | | phosphoribosyl- | | cerevisiae | lipolytica |
| | | | | (strain | | | transferase | | S288c | |
| | | | | AB0057) | | | | | | |
| YIHISMN_ | 0 | P00862 | Histidine | Lactobacillus | Bacillus | P00499 | ATP | | Salmonella | Bacillus |
| 04 | | | decarboxylase | sp. | subtilis | | phosphoribosyl- | | typhimurium | subtilis |
| | | | | (strain 30a) | | | transferase | | (strain LT2/ | |
| | | | | | | | | | SGSC1412/ | |
| | | | | | | | | | ATCC 700720) | |
| YIHISMN_ | 32011 | P00862 | Histidine | Lactobacillus | Saccharo- | P00499 | ATP | | Salmonella | Saccharo- |
| 05 | | | decarboxylase | sp. | myces | | phosphoribosyl- | | typhimurium | myces |
| | | | | (strain 30a) | cerevisiae | | transferase | | (strain LT2/ | cerevisiae |
| | | | | | | | | | SGSC1412/ | |
| | | | | | | | | | ATCC 700720) | |
| YIHISMN_ | 833 | P00862 | Histidine | Lactobacillus | Yarrowia | P00499 | ATP | | Salmonella | Yarrowia |
| 06 | | | decarboxylase | sp. | lipolytica | | phosphoribosyl- | | typhimurium | lipolytica |
| | | | | (strain 30a) | | | transferase | | (strain LT2/ | |
| | | | | | | | | | SGSC1412/ | |
| | | | | | | | | | ATCC 700720) | |
| YIHISMN_ | 299 | A0A0J6 | Histidine | Chromo- | Bacillus | Q9Z472 | ATP | N215K, | Corynebacterium | Bacillus |
| 07 | | KM89 | decarboxylase | bacterium | subtilis | | phosphoribosyl- | L231F, | glutamicum | subtilis |
| | | | | sp. LK1 | | | transferase | T235A | ATCC 13032 | |
| YIHISMN_ | 1778 | A0A0J6 | Histidine | Chromo- | Saccharo- | Q9Z472 | ATP | N215K, | Corynebacterium | Saccharo- |
| 08 | | KM89 | decarboxylase | bacterium | myces | | phosphoribosyl- | L231F, | glutamicum | myces |
| | | | | sp. LK1 | cerevisiae | | transferase | T235A | ATCC 13032 | cerevisiae |
| YIHISMN_ | 257949 | A0A0J6 | Histidine | Chromo- | Yarrowia | Q9Z472 | ATP | N215K, | Corynebacterium | Yarrowia |
| 09 | | KM89 | decarboxylase | bacterium | lipolytica | | phosphoribosyl- | L231F, | glutamicum | lipolytica |
| | | | | sp. LK1 | | | transferase | T235A | ATCC 13032 | |
| YIHISMN_ | 0 | B71459 | Histidine | Acinetobacter | Bacillus | P00499 | ATP | | Salmonella | Bacillus |
| 10 | | | decarboxylase | baumannii | subtilis | | phosphoribosyl- | | typhimurium | subtilis |
| | | | | (strain | | | transferase | | LT2 | |
| | | | | AB0057) | | | | | | |
| YIHISMN_ | 96836 | B71459 | Histidine | Acinetobacter | modified | P00499 | ATP | | Salmonella | modified |
| 11 | | | decarboxylase | baumannii | codon | | phosphoribosyl- | | typhimurium | codon |
| | | | | (strain | usage for | | transferase | | LT2 | usage for |
| | | | | AB0057) | Cg and Sc | | | | | Cg and Sc |
| YIHISMN_ | 33 | B71459 | Histidine | Acinetobacter | Saccharo- | P00499 | ATP | | Salmonella | Saccharo- |
| 12 | | | decarboxylase | baumannii | myces | | phosphoribosyl- | | typhimurium | myces |
| | | | | (strain | cerevisiae | | transferase | | LT2 | cerevisiae |
| | | | | AB0057) | | | | | | |
| YIHISMN_ | 366139 | B71459 | Histidine | Acinetobacter | Yarrowia | P00499 | ATP | | Salmonella | Yarrowia |
| 13 | | | decarboxylase | baumannii | lipolytica | | phosphoribosyl- | | typhimurium | lipolytica |
| | | | | (strain | | | transferase | | LT2 | |
| | | | | AB0057) | | | | | | |
| YIHISMN_ | 23 | P00862 | Histidine | Lactobacillus | Bacillus | Q9Z472 | ATP | N215K, | Corynebacterium | Bacillus |
| 14 | | | decarboxylase | sp. | subtilis | | phosphoribosyl- | L231F, | glutamicum | subtilis |
| | | | | (strain 30a) | | | transferase | T235A | ATCC 13032 | |
| YIHISMN_ | 26 | P00862 | Histidine | Lactobacillus | modified | Q9Z472 | ATP | N215K, | Corynebacterium | modified |
| 15 | | | decarboxylase | sp. | codon | | phosphoribosyl | L231F, | glutamicum | codon |
| | | | | (strain 30a) | usage for | | transferase | T235A | ATCC 13032 | usage for |
| | | | | | Cg and Sc | | | | | Cg and Sc |
| YIHISMN_ | 56 | P00862 | Histidine | Lactobacillus | Saccharo- | Q9Z472 | ATP | N215K, | Corynebacterium | Saccharo- |
| 16 | | | decarboxylase | sp. | myces | | phosphoribosyl- | L231F, | glutamicum | myces |
| | | | | (strain 30a) | cerevisiae | | transferase | T235A | ATCC 13032 | cerevisiae |
| YIHISMN_ | 1406 | P00862 | Histidine | Lactobacillus | Yarrowia | Q9Z472 | ATP | N215K, | Corynebacterium | Yarrowia |
| 17 | | | decarboxylase | sp. | lipolytica | | phosphoribosyl- | L231F, | glutamicum | lipolytica |
| | | | | (strain 30a) | | | transferase | T235A | ATCC 13032 | |
| YIHISMN_ | | A0A0J6 | Histidine | Chromo- | Bacillus | P00498 | ATP | | Saccharomyces | Bacillus |
| 18 | | KM89 | decarboxylase | bacterium | subtilis | | phosphoribosyl- | | cerevisiae | subtilis |
| | | | | sp. LK1 | | | transferase | | S288c | |
| YIHISMN_ | 90046 | A0A0J6 | Histidine | Chromo- | modified | P00498 | ATP | | Saccharomyces | modified |
| 19 | | KM89 | decarboxylase | bacterium | codon | | phosphoribosyl- | | cerevisiae | codon |
| | | | | sp. LK1 | usage for | | transferase | | S288c | usage for |
| | | | | | Cg and Sc | | | | | Cg and Sc |
| YIHISMN_ | 1639 | A0A0J6 | Histidine | Chromo- | Saccharo- | P00498 | ATP | | Saccharomyces | Saccharo- |
| 20 | | KM89 | decarboxylase | bacterium | myces | | phosphoribosyl- | | cerevisiae | myces |
| | | | | sp. LK1 | cerevisiae | | transferase | | S288c | cerevisiae |

TABLE 5
First-round results for production of histamine in Bacillus subtilis

| | | E1 | Enzyme 1- | Enzyme 1- | E1 Codon | E2 | Enzyme 2- | E2 | Enzyme 2- | E2 Codon |
|---|---|---|---|---|---|---|---|---|---|---|
| Strain | Titer | Uniprot | activity | source | Optimization | Uniprot | activity | Modifi- | source | Optimization |
| name | (μg/L) | ID | name | organism | Abbrev. | ID | name | cations | organism | Abbrev. |
| BsHISMN_ | | B71459 | Histidine | Acinetobacter | Yarrowia | P00498 | ATP | | Saccharomyces | Yarrowia |
| 01 | | | decarboxylase | baumannii | lipolytica | | phosphoribosyl- | | cerevisiae | lipolytica |
| | | | | (strain | | | transferase | | S288c | |
| | | | | AB0057) | | | | | | |
| BsHISMN_ | 919.7 | P00862 | Histidine | Lactobacillus | Yarrowia | P00499 | ATP | | Salmonella | Yarrowia |
| 02 | | | decarboxylase | sp. | lipolytica | | phosphoribosyl- | | typhimurium | lipolytica |
| | | | | (strain 30a) | | | transferase | | LT2 | |
| BsHISMN_ | 2.4 | A0A0J6 | Histidine | Chromo- | modified | Q9Z472 | ATP | N215K, | Corynebacterium | modified |
| 03 | | KM89 | decarboxylase | bacterium | codon | | phosphoribosyl- | L231F, | glutamicum | codon |
| | | | | sp. LK1 | usage for | | transferase | T235A | ATCC 13032 | usage for |
| | | | | | Cg and Sc | | | | | Cg and Sc |
| BsHISMN_ | 9156.1 | P00862 | Histidine | Lactobacillus | Bacillus | Q9Z472 | ATP | N215K, | Corynebacterium | Bacillus |
| 04 | | | decarboxylase | sp. | subtilis | | phosphoribosyl- | L231F, | glutamicum | subtilis |
| | | | | (strain 30a) | | | transferase | T235A | ATCC 13032 | |
| BsHISMN_ | 5057.2 | P00862 | Histidine | Lactobacillus | modified | Q9Z472 | ATP | N215K, | Corynebacterium | modified |
| 05 | | | decarboxylase | sp. | codon | | phosphoribosyl | L231F, | glutamicum | codon |
| | | | | (strain 30a) | usage for | | transferase | T235A | ATCC 13032 | usage for |
| | | | | | Cg and Sc | | | | | Cg and Sc |
| BsHISMN_ | | P00862 | Histidine | Lactobacillus | Yarrowia | Q9Z472 | ATP | N215K, | Corynebacterium | Yarrowia |
| 06 | | | decarboxylase | sp. | lipolytica | | phosphoribosyl- | L231F, | glutamicum | lipolytica |
| | | | | (strain 30a) | | | transferase | T235A | ATCC 13032 | |
| BsHISMN_ | 2532.4 | B71459 | Histidine | Acinetobacter | Bacillus | P00498 | ATP | | Saccharomyces | Bacillus |
| 07 | | | decarboxylase | baumannii | subtilis | | phosphoribosyl- | | cerevisiae | subtilis |
| | | | | (strain | | | transferase | | S288c | |
| | | | | AB0057) | | | | | | |
| BsHISMN_ | 13183.4 | B71459 | Histidine | Acinetobacter | modified | P00498 | ATP | | Saccharomyces | modified |
| 08 | | | decarboxylase | baumannii | codon | | phosphoribosyl- | | cerevisiae | codon |
| | | | | (strain | usage for | | transferase | | S288c | usage for |
| | | | | AB0057) | Cg and Sc | | | | | Cg and Sc |
| BsHISMN_ | 114.3 | B71459 | Histidine | Acinetobacter | Saccharo- | P00498 | ATP | | Saccharomyces | Saccharo- |
| 09 | | | decarboxylase | baumannii | myces | | phosphoribosyl- | | cerevisiae | myces |
| | | | | (strain | cerevisiae | | transferase | | S288c | cerevisiae |
| | | | | AB0057) | | | | | | |
| BsHISMN_ | 18336.5 | P00862 | Histidine | Lactobacillus | Bacillus | P00499 | ATP | | Salmonella | Bacillus |
| 10 | | | decarboxylase | sp. | subtilis | | phosphoribosyl- | | typhimurium | subtilis |
| | | | | (strain 30a) | | | transferase | | LT2 | |
| BsHISMN_ | 0 | P00862 | Histidine | Lactobacillus | modified | P00499 | ATP | | Salmonella | modified |
| 11 | | | decarboxylase | sp. | codon | | phosphoribosyl- | | typhimurium | codon |
| | | | | (strain 30a) | usage for | | transferase | | LT2 | usage for |
| | | | | | Cg and Sc | | | | | Cg and Sc |
| BsHISMN_ | 6778.2 | P00862 | Histidine | Lactobacillus | Saccharo- | P00499 | ATP | | Salmonella | Saccharo- |
| 12 | | | decarboxylase | sp. | myces | | phosphoribosyl- | | typhimurium | myces |
| | | | | (strain 30a) | cerevisiae | | transferase | | LT2 | cerevisiae |
| BsHISMN_ | | A0A0J6 | Histidine | Chromo- | Bacillus | Q9Z472 | ATP | N215K, | Corynebacterium | Bacillus |
| 13 | | KM89 | decarboxylase | bacterium | subtilis | | phosphoribosyl- | L231F, | glutamicum | subtilis |
| | | | | sp. LK1 | | | transferase | T235A | ATCC 13032 | |
| BsHISMN_ | 1071.1 | A0A0J6 | Histidine | Chromo- | Saccharo- | Q9Z472 | ATP | N215K, | Corynebacterium | Saccharo- |
| 14 | | KM89 | decarboxylase | bacterium | myces | | phosphoribosyl- | L231F, | glutamicum | myces |
| | | | | sp. LK1 | cerevisiae | | transferase | T235A | ATCC 13032 | cerevisiae |
| BsHISMN_ | | A0A0J6 | Histidine | Chromo- | Yarrowia | Q9Z472 | ATP | N215K, | Corynebacterium | Yarrowia |
| 15 | | KM89 | decarboxylase | bacterium | lipolytica | | phosphoribosyl- | L231F, | glutamicum | lipolytica |
| | | | | sp. LK1 | | | transferase | T235A | ATCC 13032 | |
| BsHISMN_ | 233.4 | B71459 | Histidine | Acinetobacter | Bacillus | P00499 | ATP | | Salmonella | Bacillus |
| 16 | | | decarboxylase | baumannii | subtilis | | phosphoribosyl- | | typhimurium | subtilis |
| | | | | (strain | | | transferase | | LT2 | |
| | | | | AB0057) | | | | | | |
| BsHISMN_ | 16.2 | B71459 | Histidine | Acinetobacter | modified | P00499 | ATP | | Salmonella | modified |
| 17 | | | decarboxylase | baumannii | codon | | phosphoribosyl- | | typhimurium | codon |
| | | | | (strain | usage for | | transferase | | LT2 | usage for |
| | | | | AB0057) | Cg and Sc | | | | | Cg and Sc |
| BsHISMN_ | 61 | B71459 | Histidine | Acinetobacter | Saccharo- | P00499 | ATP | | Salmonella | Saccharo- |
| 18 | | | decarboxylase | baumannii | myces | | phosphoribosyl- | | typhimurium | myces |
| | | | | (strain | cerevisiae | | transferase | | LT2 | cerevisiae |
| | | | | AB0057) | | | | | | |
| BsHISMN_ | 1413.5 | B71459 | Histidine | Acinetobacter | Yarrowia | P00499 | ATP | | Salmonella | Yarrowia |
| 19 | | | decarboxylase | baumannii | lipolytica | | phosphoribosyl- | | typhimurium | lipolytica |
| | | | | (strain | | | transferase | | LT2 | |
| | | | | AB0057) | | | | | | |
| BsHISMN_ | 6630.6 | P00862 | Histidine | Lactobacillus | Saccharo- | Q9Z472 | ATP | N215K, | Corynebacterium | Saccharo- |
| 20 | | | decarboxylase | sp. | myces | | phosphoribosyl- | L231F, | glutamicum | myces |
| | | | | (strain 30a) | cerevisiae | | transferase | T235A | ATCC 13032 | cerevisiae |
| BsHISMN_ | 43.8 | A0A0J6 | Histidine | Chromo- | Bacillus | P00498 | ATP | | Saccharomyces | Bacillus |
| 21 | | KM89 | decarboxylase | bacterium | subtilis | | phosphoribosyl- | | cerevisiae | subtilis |
| | | | | sp. LK1 | | | transferase | | S288c | |
| BsHISMN_ | | A0A0J6 | Histidine | Chromo- | modified | P00498 | ATP | | Saccharomyces | modified |
| 22 | | KM89 | decarboxylase | bacterium | codon | | phosphoribosyl- | | cerevisiae | codon |
| | | | | sp. LK1 | usage for | | transferase | | S288c | usage for |
| | | | | | Cg and Sc | | | | | Cg and Sc |
| BsHISMN_ | 529 | A0A0J6 | Histidine | Chromo- | Saccharo- | P00498 | ATP | | Saccharomyces | Saccharo- |
| 23 | | KM89 | decarboxylase | bacterium | myces | | phosphoribosyl- | | cerevisiae | myces |
| | | | | sp. LK1 | cerevisiae | | transferase | | 5288c | cerevisiae |
| BsHISMN_ | 15026.1 | A0A0J6 | Histidine | Chromo- | Yarrowia | P00498 | ATP | | Saccharomyces | Yarrowia |
| 24 | | KM89 | decarboxylase | bacterium | lipolytica | | phosphoribosyl- | | cerevisiae | lipolytica |
| | | | | sp. LK1 | | | transferase | | S288c | |
| BsHISMN_ | | A0A0J6 | Histidine | Chromo- | modified | Q9Z472 | ATP | | Corynebacterium | modified |
| 25 | | KM89 | decarboxylase | bacterium | codon | | phosphoribosyl- | | glutamicum | codon |
| | | | | sp. LK1 | usage for | | transferase | | ATCC 13032 | usage for |
| | | | | | Cg and Sc | | | | | Cg and Sc |

TABLE 6
Host evaluation designs for production of histamine tested in Saccharomyces cerevisiae

| | | | | | | | | | | |
|---|---|---|---|---|---|---|---|---|---|---|
| | | E1 | Enzyme 1- | Enzyme 1- | E1 Codon | E2 | Enzyme 2- | E2 | Enzyme 2- | E2 Codon |
| Strain | Titer | Uniprot | activity | source | Optimization | Uniprot | activity | Modifi- | source | Optimization |
| name | (μg/L) | ID | name | organism | Abbrev. | ID | name | cations | organism | Abbrev. |

Saccharomyces cerevisiae

| | | | | | | | | | | |
|---|---|---|---|---|---|---|---|---|---|---|
| ScHISMN_ | 17466 | P00862 | Histidine | Lactobacillus | Bacillus | P00499 | ATP | | Salmonella | Bacillus |
| 116 | | | decarboxylase | sp. | subtilis | | phosphoribosyl- | | typhimurium | subtilis |
| | | | | (strain 30a) | | | transferase | | LT2 | |
| ScHISMN_ | 28646 | P00862 | Histidine | Lactobacillus | Saccharo- | P00499 | ATP | | Salmonella | Saccharo- |
| 117 | | | decarboxylase | sp. | myces | | phosphoribosyl- | | typhimurium | myces |
| | | | | (strain 30a) | cerevisiae | | transferase | | LT2 | cerevisiae |
| ScHISMN_ | 48150 | P00862 | Histidine | Lactobacillus | Yarrowia | P00499 | ATP | | Salmonella | Yarrowia |
| 118 | | | decarboxylase | sp. | lipolytica | | phosphoribosyl- | | typhimurium | lipolytica |
| | | | | (strain 30a) | | | transferase | | LT2 | |
| ScHISMN_ | 59265 | A0A0J6 | Histidine | Chromo- | Bacillus | Q9Z472 | ATP | N215K, | Corynebacterium | Bacillus |
| 119 | | KM89 | decarboxylase | bacterium | subtilis | | phosphoribosyl- | L231F, | glutamicum | subtilis |
| | | | | sp. LK1 | | | transferase | T235A | ATCC 13032 | |
| ScHISMN_ | 72566 | A0A0J6 | Histidine | Chromo- | modified | Q9Z472 | ATP | N215K, | Corynebacterium | modified |
| 120 | | KM89 | decarboxylase | bacterium | codon | | phosphoribosyl | L231F, | glutamicum | codon |
| | | | | sp. LK1 | usage for | | transferase | T235A | ATCC 13032 | usage for |
| | | | | | Cg and Sc | | | | | Cg and Sc |
| ScHISMN_ | 46418 | A0A0J6 | Histidine | Chromo- | Saccharo- | Q9Z472 | ATP | N215K, | Corynebacterium | Saccharo- |
| 121 | | KM89 | decarboxylase | bacterium | myces | | phosphoribosyl- | L231F, | glutamicum | myces |
| | | | | sp. LK1 | cerevisiae | | transferase | T235A | ATCC 13032 | cerevisiae |
| ScHISMN_ | 64087 | A0A0J6 | Histidine | Chromo- | Yarrowia | Q9Z472 | ATP | N215K, | Corynebacterium | Yarrowia |
| 122 | | KM89 | decarboxylase | bacterium | lipolytica | | phosphoribosyl- | L231F, | glutamicum | lipolytica |
| | | | | sp. LK1 | | | transferase | T235A | ATCC 13032 | |
| ScHISMN_ | 80704 | B71459 | Histidine | Acinetobacter | Bacillus | P00499 | ATP | | Salmonella | Bacillus |
| 123 | | | decarboxylase | baumannii | subtilis | | phosphoribosyl- | | typhimurium | subtilis |
| | | | | (strain | | | transferase | | LT2 | |
| | | | | AB0057) | | | | | | |
| ScHISMN_ | 70043 | B71459 | Histidine | Acinetobacter | Yarrowia | P00499 | ATP | | Salmonella | Yarrowia |
| 124 | | | decarboxylase | baumannii | lipolytica | | phosphoribosyl- | | typhimurium | lipolytica |
| | | | | (strain | | | transferase | | LT2 | |
| | | | | AB0057) | | | | | | |
| ScHISMN_ | 25331 | P00862 | Histidine | Lactobacillus | Bacillus | Q9Z472 | ATP | N215K, | Corynebacterium | Bacillus |
| 125 | | | decarboxylase | sp. | subtilis | | phosphoribosyl- | L231F, | glutamicum | subtilis |
| | | | | (strain 30a) | | | transferase | T235A | ATCC 13032 | |
| ScHISMN_ | 33970 | P00862 | Histidine | Lactobacillus | modified | Q9Z472 | ATP | N215K, | Corynebacterium | modified |
| 126 | | | decarboxylase | sp. | codon | | phosphoribosyl | L231F, | glutamicum | codon |
| | | | | (strain 30a) | usage for | | transferase | T235A | ATCC 13032 | usage for |
| | | | | | Cg and Sc | | | | | Cg and Sc |
| ScHISMN_ | 21402 | P00862 | Histidine | Lactobacillus | Saccharo- | Q9Z472 | ATP | N215K, | Corynebacterium | Saccharo- |
| 127 | | | decarboxylase | sp. | myces | | phosphoribosyl- | L231F, | glutamicum | myces |
| | | | | (strain 30a) | cerevisiae | | transferase | T235A | ATCC 13032 | cerevisiae |
| ScHISMN_ | 41854 | P00862 | Histidine | Lactobacillus | Yarrowia | Q9Z472 | ATP | N215K, | Corynebacterium | Yarrowia |
| 128 | | | decarboxylase | sp. | lipolytica | | phosphoribosyl- | L231F, | glutamicum | lipolytica |
| | | | | (strain 30a) | | | transferase | T235A | ATCC 13032 | |
| ScHISMN_ | 101496 | A0A0J6 | Histidine | Chromo- | Bacillus | P00498 | ATP | | Saccharomyces | Bacillus |
| 129 | | KM89 | decarboxylase | bacterium | subtilis | | phosphoribosyl- | | cerevisiae | subtilis |
| | | | | sp. LK1 | | | transferase | | S288c | |
| ScHISMN_ | 85546 | A0A0J6 | Histidine | Chromo- | Saccharo- | P00498 | ATP | | Saccharomyces | Saccharo- |
| 130 | | KM89 | decarboxylase | bacterium | myces | | phosphoribosyl- | | cerevisiae | myces |
| | | | | sp. LK1 | cerevisiae | | transferase | | S288c | cerevisiae |
| ScHISMN_ | 111109 | A0A0J6 | Histidine | Chromo- | Yarrowia | P00498 | ATP | | Saccharomyces | Yarrowia |
| 131 | | KM89 | decarboxylase | bacterium | lipolytica | | phosphoribosyl- | | cerevisiae | lipolytica |
| | | | | sp. LK1 | | | transferase | | S288c | |

TABLE 7
Host evaluation designs for production of histamine tested in Corynebacterium glutamicum

| | | E1 | Enzyme 1- | Enzyme 1- | E1 Codon | E2 | Enzyme 2- | E2 | Enzyme 2- | E2 Codon |
|---|---|---|---|---|---|---|---|---|---|---|
| Strain | Titer | Uniprot | activity | source | Optimization | Uniprot | activity | Modifi- | source | Optimization |
| name | (μg/L) | ID | name | organism | Abbrev. | ID | name | cations | organism | Abbrev. |
| CgHISMN_ | | B71459 | Histidine | Acinetobacter | Bacillus | P00498 | ATP | | Saccharomyces | Bacillus |
| 70 | | | decarboxylase | baumannii | subtilis | | phosphoribosyl- | | cerevisiae | subtilis |
| | | | | (strain | | | transferase | | S288c | |
| | | | | AB0057) | | | | | | |
| CgHISMN_ | 68395.9 | B71459 | Histidine | Acinetobacter | modified | P00498 | ATP | | Saccharomyces | modified |
| 71 | | | decarboxylase | baumannii | codon | | phosphoribosyl- | | cerevisiae | codon |
| | | | | (strain | usage for | | transferase | | S288c | usage for |
| | | | | AB0057) | Cg and Sc | | | | | Cg and Sc |
| CgHISMN_ | 18 | B71459 | Histidine | Acinetobacter | Saccharo- | P00498 | ATP | | Saccharomyces | Saccharo- |
| 72 | | | decarboxylase | baumannii | myces | | phosphoribosyl- | | cerevisiae | myces |
| | | | | (strain | cerevisiae | | transferase | | S288c | cerevisiae |
| | | | | AB0057) | | | | | | |
| CgHISMN_ | 16325.5 | B71459 | Histidine | Acinetobacter | Yarrowia | P00498 | ATP | | Saccharomyces | Yarrowia |
| 73 | | | decarboxylase | baumannii | lipolytica | | phosphoribosyl- | | cerevisiae | lipolytica |
| | | | | (strain | | | transferase | | S288c | |
| | | | | AB0057) | | | | | | |
| CgHISMN_ | 4883.6 | P00862 | Histidine | Lactobacillus | Bacillus | P00499 | ATP | | Salmonella | Bacillus |
| 74 | | | decarboxylase | sp. | subtilis | | phosphoribosyl- | | typhimurium | subtilis |
| | | | | (strain 30a) | | | transferase | | LT2 | |
| CgHISMN_ | | P00862 | Histidine | Lactobacillus | Saccharo- | P00499 | ATP | | Salmonella | Saccharo- |
| 75 | | | decarboxylase | sp. | myces | | phosphoribosyl- | | typhimurium | myces |
| | | | | (strain 30a) | cerevisiae | | transferase | | LT2 | cerevisiae |
| CgHISMN_ | | P00862 | Histidine | Lactobacillus | Yarrowia | P00499 | ATP | | Salmonella | Yarrowia |
| 76 | | | decarboxylase | sp. | lipolytica | | phosphoribosyl- | | typhimurium | lipolytica |
| | | | | (strain 30a) | | | transferase | | LT2 | |
| CgHISMN_ | 5.4 | A0A0J6 | Histidine | Chromo- | Bacillus | Q9Z472 | ATP | N215K, | Corynebacterium | Bacillus |
| 77 | | KM89 | decarboxylase | bacterium | subtilis | | phosphoribosyl- | L231F, | glutamicum | subtilis |
| | | | | sp. LK1 | | | transferase | T235A | ATCC 13032 | |
| CgHISMN_ | 88.6 | A0A0J6 | Histidine | Chromo- | Saccharo- | Q9Z472 | ATP | N215K, | Corynebacterium | Saccharo- |
| 78 | | KM89 | decarboxylase | bacterium | myces | | phosphoribosyl- | L231F, | glutamicum | myces |
| | | | | sp. LK1 | cerevisiae | | transferase | T235A | ATCC 13032 | cerevisiae |
| CgHISMN_ | | A0A0J6 | Histidine | Chromo- | Yarrowia | Q9Z472 | ATP | N215K, | Corynebacterium | Yarrowia |
| 79 | | KM89 | decarboxylase | bacterium | lipolytica | | phosphoribosyl- | L231F, | glutamicum | lipolytica |
| | | | | sp. LK1 | | | transferase | T235A | ATCC 13032 | |
| CgHISMN_ | 8368.2 | B71459 | Histidine | Acinetobacter | Bacillus | P00499 | ATP | | Salmonella | Bacillus |
| 80 | | | decarboxylase | baumannii | subtilis | | phosphoribosyl- | | typhimurium | subtilis |
| | | | | (strain | | | | | LT2 | |
| | | | | AB0057) | | | | | | |
| CgHISMN_ | 9.3 | B71459 | Histidine | Acinetobacter | Saccharo- | P00499 | ATP | | Salmonella | Saccharo- |
| 81 | | | decarboxylase | baumannii | myces | | phosphoribosyl- | | typhimurium | myces |
| | | | | (strain | cerevisiae | | transferase | | LT2 | cerevisiae |
| | | | | AB0057) | | | | | | |
| CgHISMN_ | 15529.4 | B71459 | Histidine | Acinetobacter | Yarrowia | P00499 | ATP | | Salmonella | Yarrowia |
| 82 | | | decarboxylase | baumannii | lipolytica | | phosphoribosyl- | | typhimurium | lipolytica |
| | | | | (strain | | | transferase | | LT2 | |
| | | | | AB0057) | | | | | | |
| CgHISMN_ | | P00862 | Histidine | Lactobacillus | Bacillus | Q9Z472 | ATP | N215K, | Corynebacterium | Bacillus |
| 83 | | | decarboxylase | sp. | subtilis | | phosphoribosyl- | L231F, | glutamicum | subtilis |
| | | | | (strain 30a) | | | transferase | T235A | ATCC 13032 | |
| CgHISMN_ | 2.6 | P00862 | Histidine | Lactobacillus | Saccharo- | Q9Z472 | ATP | N215K, | Corynebacterium | Saccharo- |
| 84 | | | decarboxylase | sp. | myces | | phosphoribosyl- | L231F, | glutamicum | myces |
| | | | | (strain 30a) | cerevisiae | | transferase | T235A | ATCC 13032 | cerevisiae |
| CgHISMN_ | 6134 | P00862 | Histidine | Lactobacillus | Yarrowia | Q9Z472 | ATP | N215K, | Corynebacterium | Yarrowia |
| 85 | | | decarboxylase | sp. | lipolytica | | phosphoribosyl- | L231F, | glutamicum | lipolytica |
| | | | | (strain 30a) | | | transferase | T235A | ATCC 13032 | |
| CgHISMN_ | 197 | A0A0J6 | Histidine | Chromo- | Bacillus | P00498 | ATP | | Saccharomyces | Bacillus |
| 86 | | KM89 | decarboxylase | bacterium | subtilis | | phosphoribosyl- | | cerevisiae | subtilis |
| | | | | sp. LK1 | | | transferase | | S288c | |

TABLE 8
SEQ ID NO Cross-Reference Table

| SEQ | | | | | |
|---|---|---|---|---|---|
| ID | Sequence Type with | Uniprot | | | Codon |
| NO | Modifications | ID | Activity name | Source organism | Optimization Abbrev. |
| 1 | AA seq for | B71459 | histidine decarboxylase | Acinetobacter baumannii | |
| | enzyme B71459 | | | (strain AB0057) | |
| 2 | AA seq for | Q9KJU3 | Imidazoleglycerol- | Corynebacterium glutamicum | |
| | enzyme Q9KJU3 | | phosphate dehydratase | | |
| 3 | AA seq for | P00498 | ATP phosphoribosyltransferase | Saccharomyces cerevisiae | |
| | enzyme P00498 | | | | |
| 4 | AA seq for | P00862 | histidine decarboxylase | Lactobacillus sp. (strain 30a) | |
| | enzyme P00862 | | | | |
| 5 | AA seq for | P00499 | ATP phosphoribosyltransferase | Salmonella typhimurium | |
| | enzyme P00499 with | | | (strain LT2/SGSC1412/ | |
| | deletion of Q207-E208 | | | ATCC 700720) | |
| 6 | AA seq for | J6KM89 | histidine decarboxylase | Chromobacterium sp. LK1 | |
| | enzyme J6KM89 | | | | |
| 7 | AA seq for | Q9Z472 | ATP phosphoribosyltransferase | Corynebacterium glutamicum | |
| | enzyme Q9Z472 | | | (strain ATCC 13032/ | |
| | with substitution | | | DSM 20300/JCM 1318/ | |
| | N215K, L231F, T235A | | | LMG 3730/NCIMB 10025) | |
| 8 | AA seq for | E3QMN8 | histidine decarboxylase | Methanosarcina barkeri | |
| | enzyme E3QMN8 | | | str. Wiesmoor | |
| 9 | AA seq for | Q467R8 | histidine decarboxylase | Methanosarcina barkeri | |
| | enzyme Q467R8 | | | (strain Fusaro/DSM 804) | |
| 10 | AA seq for | Q05733 | histidine decarboxylase | Drosophila melanogaster | |
| | enzyme Q05733 | | | | |
| 11 | AA seq for | P54772 | histidine decarboxylase | Solanum lycopersicum | |
| | enzyme P54772 | | | | |
| 12 | AA seq for | P23738 | histidine decarboxylase | Mus musculus | |
| | enzyme P23738 | | | | |
| 13 | AA seq for | O68602 | 1-(5-phosphoribosyl)5[(5- | Corynebacterium glutamicum | |
| | enzyme O68602 | | phosphoribosylamino) | | |
| | | | methylideneamino] | | |
| | | | imidazole-4- | | |
| | | | carboxamide isomerase | | |
| 14 | AA seq for | Q9KJU4 | Histidinol-phosphate | Corynebacterium glutamicum | |
| | enzyme Q9KJU4 | | aminotransferase | | |
| 15 | AA seq for | Q8NNT5 | Histidinol dehydrogenase | Corynebacterium glutamicum | |
| | enzyme Q8NNT5 | | | | |
| 16 | AA seq for | Q9Z471 | Phosphoribosyl- | Corynebacterium glutamicum | |
| | enzyme Q9Z471 | | ATP pyrophosphatase | | |
| 17 | AA seq for | O31139 | Imidazole glycerol phosphate | Corynebacterium glutamicum | |
| | enzyme O31139 | | synthase subunit | | |
| 18 | AA seq for | O69043 | Imidazole glycerol phosphate | Corynebacterium glutamicum | |
| | enzyme O69043 | | synthase subunit | | |
| 19 | AA seq for | Q8NNT9 | phosphoribosyl- | Corynebacterium glutamicum | |
| | enzyme Q8NNT9 | | AMP cyclohydrolase | | |
| 22 | AA seq for | Q9Z472 | ATP phosphoribosyltransferase | Corynebacterium glutamicum | |
| | enzyme Q9Z472 | | | (strain ATCC 13032/ | |
| | | | | DSM 20300/JCM 1318/ | |
| | | | | LMG 3730/NCIMB 10025) | |
| 20 | AA seq for | P00815 | histidinol dehydrogenase, | Saccharomyces cerevisiae | |
| | enzyme P00815 | | phosphoribosyl- | | |
| | | | AMP cyclohydrolase, | | |
| | | | phosphoribosyl- | | |
| | | | ATP diphosphatase | | |
| 21 | AA seq for | P33734 | Imidazole glycerol phosphate | Saccharomyces cerevisiae | |
| | enzyme P33734 | | synthase subunit HisF | | |
| 23 | AA seq for | P07172 | histidinol- | Saccharomyces cerevisiae | |
| | enzyme P07172 | | phosphate transaminase | | |
| 24 | AA seq for | P06633 | Imidazoleglycerol- | Saccharomyces cerevisiae | |
| | enzyme P06633 | | phosphate dehydratase | | |
| 25 | AA seq for | P38635 | histidinol-phosphatase | Saccharomyces cerevisiae | |
| | enzyme P38635 | | | | |
| 26 | AA seq for | A4QEF2 | Glucose-6-phosphate | Corynebacterium glutamicum | |
| | enzyme A4QEF2 with | | 1-dehydrogenase | | |
| | substitution A243T | | (G6PD) (EC 1.1.1.49) | (strain R) | |
| 27 | AA seq for | P23254 | Transketolase | Saccharomyces cerevisiae | |
| | enzyme P23254 | | | (strain ATCC 204508/S288c) | |
| | | | | (Baker's yeast) | |
| 28 | AA seq for | Q12265 | Ribose-phosphate | Saccharomyces cerevisiae | |
| | enzyme Q12265 | | pyrophosphokinase 5 | (strain ATCC 204508/S288c) | |
| | | | (EC 2.7.6.1) | (Baker's yeast) | |
| | | | (Phosphoribosyl | | |
| | | | pyrophosphate synthase 5) | | |
| 30 | DNA seq1 for | Q9Z472 | ATP phosphoribosyltransferase | Corynebacterium glutamicum | native |
| | enzyme Q9Z472 | | | | |
| 29 | DNA seq1 for | P00815 | histidinol dehydrogenase, | Saccharomyces cerevisiae | native |
| | enzyme P00815 | | phosphoribosyl- | | |
| | | | AMP cyclohydrolase, | | |
| | | | phosphoribosyl- | | |
| | | | ATP diphosphatase | | |
| 31 | AA seq for | P40545 | 1-(5-phosphoribosyl)-5-[(5- | Saccharomyces cerevisiae | |
| | enzyme P40545 | | phosphoribosylamino) | (strain ATCC 204508/S288c) | |
| | | | methylideneamino] | (Baker's yeast) | |
| | | | imidazole-4-carboxamide | | |
| | | | isomerase (EC 5.3.1.16) | | |
| | | | (5-proFAR isomerase) | | |
| | | | (Phosphoribosylformimino-5- | | |
| | | | aminoimidazole carboxamide | | |
| | | | ribotide isomerase) | | |
| 32 | AA seq for | P00942 | Triosephosphate isomerase | Saccharomyces cerevisiae | |
| | enzyme P00942 with | | (TIM) (EC 5.3.1.1) | (strain ATCC 204508/S288c) | |
| | substitution 1170V | | (Triose-phosphate isomerase) | (Baker's yeast) | |
| 33 | AA seq for enzyme | A0A089 | Histidine decarboxylase | Pseudomonas rhizosphaerae | |
| | A0A089YPE5 | YPE5 | (HDC) (EC 4.1.1.22) | | |
| 34 | AA seq for enzyme | A0A126 | Histidine decarboxylase | Pseudomonas putida | |
| | AOA12656G9 | 56G9 | (HDC) (EC 4.1.1.22) | (Arthrobacter siderocapsulatus) | |
| 35 | AA seq for enzyme | A0A0A1 | Histidine decarboxylase | Citrobacter pasteurii | |
| | A0A0A1R6V3 | R6V3 | (HDC) (EC 4.1.1.22) | | |
| 36 | AA seq for enzyme | A0A1W0 | Histidine decarboxylase | Chromobacterium haemolyticum | |
| | A0A1W0CM88 | CM88 | (HDC) (EC 4.1.1.22) | | |
| 37 | AA seq for enzyme | A0A0K6 | Histidine decarboxylase | Lactobacillus reuteri | |
| | A0A0K6GJ74 | GJ74 | proenzyme | | |
| 38 | AA seq for | T0QL99 | Histidine decarboxylase | Aeromonas salmonicida | |
| | enzyme T0QL99 | | (EC 4.1.1.22) (Fragment) | subsp. pectinolytica 34mel | |
| 39 | AA seq for enzyme | A0A1B8 | Histidine decarboxylase | Morganella psychrotolerans | |
| | A0A1B8HLR1 | HLR1 | (HDC) (EC 4.1.1.22) | | |
| 40 | AA seq for enzyme | A0A0C1 | Histidine decarboxylase | Lactobacillus fructivorans | |
| | A0A0C1PR48 | PR48 | proenzyme | | |
| 41 | AA seq for | P0A717 | Ribose-phosphate | Escherichia coli (strain K12) | |
| | enzyme P0A717 | | pyrophosphokinase | | |
| | | | (RPPK) (EC 2.7.6.1) | | |
| | | | (5-phospho-D-ribosyl | | |
| | | | alpha-1-diphosphate) | | |
| | | | (Phosphoribosyl | | |
| | | | diphosphate synthase) | | |
| | | | (Phosphoribosyl | | |
| | | | pyrophosphate synthase) | | |
| | | | (P-Rib-PP synthase) | | |
| | | | (PRPP synthase) (PRPPase) | | |
| 42 | AA seq for | Q680A5 | Ribose-phosphate | Arabidopsis thaliana | |
| | enzyme Q680A5 | | pyrophosphokinase 4 | (Mouse-ear cress) | |
| | | | (EC 2.7.6.1) | | |
| | | | (Phosphoribosyl | | |
| | | | pyrophosphate synthase 4) | | |
| 43 | AA seq for | P38620 | Ribose-phosphate | Saccharomyces cerevisiae | |
| | enzyme P38620 | | pyrophosphokinase 2 | (strain ATCC 204508/S288c) | |
| | | | (EC 2.7.6.1) | (Baker's yeast) | |
| | | | (Phosphoribosyl | | |
| | | | pyrophosphate synthase 2) | | |
| 44 | AA seq for | P32895 | Ribose-phosphate | Saccharomyces cerevisiae | |
| | enzyme P32895 | | pyrophosphokinase 1 | (strain ATCC 204508/S288c) | |
| | | | (EC 2.7.6.1) | (Baker's yeast) | |
| | | | (Phosphoribosyl | | |
| | | | pyrophosphate synthase 1) | | |
| 45 | AA seq for | P38689 | Ribose-phosphate | Saccharomyces cerevisiae | |
| | enzyme P38689 | | pyrophosphokinase 3 | (strain ATCC 204508/S288c) | |
| | | | (EC 2.7.6.1) | (Baker's yeast) | |
| | | | (Phosphoribosyl | | |
| | | | pyrophosphate synthase 3) | | |
| 46 | AA seq for | P15019 | Transaldolase (EC 2.2.1.2) | Saccharomyces cerevisiae | |
| | enzyme P15019 | | | (strain ATCC 204508/S288c) | |
| | | | | (Baker's yeast) | |
| 47 | AA seq for | P06775 | Histidine permease | Saccharomyces cerevisiae | |
| | enzyme P06775 | | | (strain ATCC 204508/S288c) | |
| | | | | (Baker's yeast) | |
| 48 | AA seq for | O59667 | Histidine biosynthesis | Schizosaccharomyces pombe | |
| | enzyme O59667 | | bifunctional protein | (strain 972/ATCC 24843) | |
| | | | his7 [Includes: | (Fission yeast) | |
| | | | Phosphoribosyl- | | |
| | | | AMP cyclohydrolase | | |
| | | | (EC 3.5.4.19); | | |
| | | | Phosphoribosyl- | | |
| | | | ATP pyrophosphatase | | |
| | | | (EC 3.6.1.31)] | | |
| 49 | AA seq for | O66000 | Histidine decarboxylase | Oenococcus oeni | |
| | enzyme O66000 | | proenzyme | (Leuconostoc oenos) | |
| 50 | AA seq for enzyme | A0A0R1 | Pyruvoyl family | Lactobacillus aviarius subsp. | |
| | A0A0R1Y874 | Y874 | histidine decarboxylase | aviarius DSM 20655 | |
| 51 | AA seq for enzyme | A0A0J6K | Histidine decarboxylase | Chromobacterium sp. LK1 | |
| | A0A0J6KM89 | M89 | (HDC) (EC 4.1.1.22) | | |
| 52 | DNA seq1 for | B71459 | Histidine decarboxylase | Acinetobacter baumannii | Yarrowia lipolytica |
| | enzyme B71459 | | | (strain AB0057) | |
| 53 | DNA seq1 for | P00498 | ATP phosphoribosyltransferase | Saccharomyces cerevisiae | Yarrowia lipolytica |
| | enzyme P00498 | | | (strain ATCC 204508/S288c) | |
| | | | | (Baker's yeast) | |
| 54 | DNA seq2 for | B71459 | Histidine decarboxylase | Acinetobacter baumannii | Bacillus subtillus |
| | enzyme B71459 | | | (strain AB0057) | |
| 55 | DNA seq2 for | P00498 | ATP phosphoribosyltransferase | Saccharomyces cerevisiae | Bacillus subtillus |
| | enzyme P00498 | | | (strain ATCC 204508/S288c) | |
| | | | | (Baker's yeast) | |
| 56 | DNA seq3 for | B71459 | Histidine decarboxylase | Acinetobacter baumannii | Saccharomyces cerevisiae |
| | enzyme B71459 | | | (strain AB0057) | |
| 57 | DNA seq3 for | P00498 | ATP phosphoribosyltransferase | Saccharomyces cerevisiae | Saccharomyces cerevisiae |
| | enzyme P00498 | | | (strain ATCC 204508/S288c) | |
| | | | | (Baker's yeast) | |
| 58 | DNA seq1 for | P00499 | ATP phosphoribosyltransferase | Salmonella typhimurium | Yarrowia lipolytica |
| | enzyme P00499 with | | | (strain LT2/SGSC1412/ | |
| | deletion of Q207-E208 | | | ATCC 700720) | |
| 59 | DNA seq2 for | P00499 | ATP phosphoribosyltransferase | Salmonella typhimurium | Bacillus subtillus |
| | enzyme P00499 with | | | (strain LT2/SGSC1412/ | |
| | deletion of Q207-E208 | | | ATCC 700720) | |
| 60 | DNA seq3 for | P00499 | ATP phosphoribosyltransferase | Salmonella typhimurium | Saccharomyces cerevisiae |
| | enzyme P00499 with | | | (strain LT2/SGSC1412/ | |
| | deletion of Q207-E208 | | | ATCC 700720) | |
| 61 | DNA seq4 for | B71459 | Histidine decarboxylase | Acinetobacter baumannii | modified codon usage for |
| | enzyme B71459 | | (HDC) (EC 4.1.1.22) | (strain AB0057) | Corynebacterium glutamicum and |
| | | | | | Saccharomyces cerevisiae |
| 62 | DNA seq4 for | P00499 | ATP phosphoribosyltransferase | Salmonella typhimurium | modified codon usage for |
| | enzyme P00499 with | | | (strain LT2/SGSC1412/ | Corynebacterium glutamicum and |
| | deletion of Q207-E208 | | | ATCC 700720) | Saccharomyces cerevisiae |
| 63 | DNA seq1 for enzyme | A0A0J6K | Histidine decarboxylase | Chromobacterium sp. LK1 | Yarrowia lipolytica |
| | A0A0J6KM89 | M89 | | | |
| 64 | DNA seq1 | Q9Z472 | ATP phosphoribosyltransferase | Corynebacterium glutamicum | Yarrowia lipolytica |
| | for enzyme Q9Z472 | | | (strain ATCC 13032/ | |
| | with substitution | | | DSM 20300/JCM 1318/ | |
| | N215K, L231F, T235A | | | LMG 3730/NCIMB 10025) | |
| 65 | DNA seq2 for enzyme | A0A0J6K | Histidine decarboxylase | Chromobacterium sp. LK1 | Saccharomyces cerevisiae |
| | A0A0J6KM89 | M89 | | | |
| 66 | DNA seq2 | Q9Z472 | ATP phosphoribosyltransferase | Corynebacterium glutamicum | Saccharomyces cerevisiae |
| | for enzyme Q9Z472 | | | (strain ATCC 13032/ | |
| | with substitution | | | DSM 20300/JCM 1318/ | |
| | N215K, L231F, T235A | | | LMG 3730/NCIMB 10025) | |
| 67 | DNA seq3 | A0A0J6K | Histidine decarboxylase | Chromobacterium sp. LK1 | Bacillus subtillus |
| | for enzyme | M89 | | | |
| | A0A0J6KM89 | | | | |
| 68 | DNA seq3 | Q9Z472 | ATP phosphoribosyltransferase | Corynebacterium glutamicum | Bacillus subtillus |
| | for enzyme Q9Z472 | | | (strain ATCC 13032/ | |
| | with substitution | | | DSM 20300/JCM 1318/ | |
| | N215K, L231F, T235A | | | LMG 3730/NCIMB 10025) | |
| 69 | DNA seq1 for | P00862 | Histidine decarboxylase | Lactobacillus sp. (strain 30a) | Bacillus subtillus |
| | enzyme P00862 | | proenzyme | | |
| 70 | DNA seq2 for | P00862 | Histidine decarboxylase | Lactobacillus sp. (strain 30a) | Saccharomyces cerevisiae |
| | enzyme P00862 | | proenzyme | | |
| 71 | DNA seq3 for | P00862 | Histidine decarboxylase | Lactobacillus sp. (strain 30a) | modified codon usage for |
| | enzyme P00862 | | proenzyme | | Corynebacterium glutamicum and |
| | | | | | Saccharomyces cerevisiae |
| 72 | DNA seq5 for | B71459 | Histidine decarboxylase | Acinetobacter baumannii | modified codon usage for |
| | enzyme B71459 | | | (strain AB0057) | Corynebacterium glutamicum and |
| | | | | | Saccharomyces cerevisiae |
| 73 | DNA seq4 for | P00498 | ATP phosphoribosyltransferase | Saccharomyces cerevisiae | modified codon usage for |
| | enzyme P00498 | | | (strain ATCC 204508/S288c) | Corynebacterium glutamicum and |
| | | | | (Baker's yeast) | Saccharomyces cerevisiae |
| 74 | AA seq for enzyme | A0A1H1 | Histidine decarboxylase | Pseudomonas sp. bs2935 | modified codon usage for |
| | A0A1H1TEB8 | TEB8 | (HDC) (EC 4.1.1.22) | | Corynebacterium glutamicum and |
| | | | | | Saccharomyces cerevisiae |
| 75 | DNA seq1 for | E3QMN8 | histidine decarboxylase | Methanosarcina barkeri | Corynebacterium glutamicum |
| | enzyme E3QMN8 | | | str. Wiesmoor | |
| 76 | DNA seq1 for | Q467R8 | histidine decarboxylase | Methanosarcina barkeri | Corynebacterium glutamicum |
| | enzyme Q467R8 | | | (strain Fusaro/DSM 804) | |
| 77 | DNA seq4 for | P00862 | histidine decarboxylase | Lactobacillus sp. (strain 30a) | Corynebacterium glutamicum |
| | enzyme P00862 | | | | |
| 78 | DNA seq6 for | B71459 | histidine decarboxylase | Acinetobacter baumannii | Corynebacterium glutamicum |
| | enzyme B71459 | | | (strain AB0057) | |
| 79 | DNA seq1 for | Q05733 | histidine decarboxylase | Drosophila melanogaster | Corynebacterium glutamicum |
| | enzyme Q05733 | | | | |
| 80 | DNA seq1 for | J6KM89 | histidine decarboxylase | Chromobacterium sp. LK1 | Corynebacterium glutamicum |
| | enzyme J6KM89 | | | | |
| 81 | DNA seq5 for | P00499 | ATP phosphoribosyltransferase | Salmonella typhimurium | Corynebacterium glutamicum |
| | enzyme P00499 with | | | (strain LT2/SGSC1412/ | |
| | deletion of Q207-E208 | | | ATCC 700720) | |
| 82 | DNA seq5 for | P00862 | histidine decarboxylase | Lactobacillus sp. (strain 30a) | Saccharomyces cerevisiae |
| | enzyme P00862 | | | | |
| 83 | DNA seq for | P54772 | histidine decarboxylase | Solanum lycopersicum | Saccharomyces cerevisiae |
| | enzyme P54772 | | | | |
| 84 | DNA seq for | P23738 | histidine decarboxylase | Mus musculus | Saccharomyces cerevisiae |
| | enzyme P23738 | | | | |
| 85 | DNA seq2 for | Q05733 | histidine decarboxylase | Drosophila melanogaster | Saccharomyces cerevisiae |
| | enzyme Q05733 | | | | |
| 86 | DNA seq2 for | J6KM89 | histidine decarboxylase | Chromobacterium sp. LK1 | Saccharomyces cerevisiae |
| | enzyme J6KM89 | | | | |
| 87 | DNA seq2 for | E3QMN8 | histidine decarboxylase | Methanosarcina barkeri | Saccharomyces cerevisiae |
| | enzyme E3QMN8 | | | str. Wiesmoor | |
| 88 | DNA seq2 for | Q467R8 | histidine decarboxylase | Methanosarcina barkeri | Saccharomyces cerevisiae |
| | enzyme Q467R8 | | | (strain Fusaro/DSM 804) | |
| 89 | DNA seq4 | Q9Z472 | ATP phosphoribosyltransferase | Corynebacterium glutamicum | Saccharomyces cerevisiae |
| | for enzyme Q9Z472 | | | (strain ATCC 13032/ | |
| | with substitution | | | DSM 20300/JCM 1318/ | |
| | N215K, L231F, T235A | | | LMG 3730/NCIMB 10025) | |
| 90 | DNA seq6 for | P00499 | ATP phosphoribosyltransferase | Salmonella typhimurium | Saccharomyces cerevisiae |
| | enzyme P00499 with | | | (strain LT2/SGSC1412/ | |
| | deletion of Q207-E208 | | | ATCC 700720) | |
| 91 | DNA seq for | O68602 | 1-(5-phosphoribosyl)5[(5- | Corynebacterium glutamicum | native |
| | enzyme O68602 | | phosphoribosylamino) | | |
| | | | methylideneamino]imidazole- | | |
| | | | 4-carboxamide isomerase | | |
| 92 | DNA seq for | Q9KJU3 | Imidazoleglycerol- | Corynebacterium glutamicum | native |
| | enzyme Q9KJU3 | | phosphate dehydratase | | |
| 93 | DNA seq for | Q9KJU4 | Histidinol-phosphate | Corynebacterium glutamicum | native |
| | enzyme Q9KJU4 | | aminotransferase | | |
| 94 | DNA seq for | Q8NNT5 | Histidinol dehydrogenase | Corynebacterium glutamicum | native |
| | enzyme Q8NNT5 | | | | |
| 95 | DNA seq for | Q9Z471 | Phosphoribosyl-ATP | Corynebacterium glutamicum | native |
| | enzyme Q9Z471 | | pyrophosphatase | | |
| 96 | DNA seq for | O31139 | Imidazole glycerol phosphate | Corynebacterium glutamicum | native |
| | enzyme O31139 | | synthase subunit | | |
| 97 | DNA seq for | O69043 | Imidazole glycerol phosphate | Corynebacterium glutamicum | native |
| | enzyme O69043 | | synthase subunit | | |
| 98 | DNA seq for | Q8NNT9 | phosphoribosyl- | Corynebacterium glutamicum | native |
| | enzyme Q8NNT9 | | AMP cyclohydrolase | | |
| 99 | DNA seq5 for | P00498 | ATP phosphoribosyltransferase | Saccharomyces cerevisiae | native |
| | enzyme P00498 | | Imidazole glycerol phosphate | | |
| 100 | DNA seq1 for | P33734 | synthase subunit HisF | Saccharomyces cerevisiae | native |
| | enzyme P33734 | | | | |
| 101 | DNA seq1 for | P07172 | histidinol-phosphate | Saccharomyces cerevisiae | native |
| | enzyme P07172 | | transaminase | | |
| 102 | DNA seq for | P06633 | Imidazoleglycerol- | Saccharomyces cerevisiae | native |
| | enzyme P06633 | | phosphate dehydratase | | |
| 103 | DNA seq1 for | P38635 | histidinol-phosphatase | Saccharomyces cerevisiae | native |
| | enzyme P38635 | | | | |
| 104 | DNA seq | A0A0C1 | Histidine decarboxylase | Lactobacillus fructivorans | modified codon usage for |
| | for enzyme | PR48 | proenzyme | | Corynebacterium glutamicum and |
| | A0A0C1PR48 | | | | Saccharomyces cerevisiae |
| 105 | DNA seq for | T0QL99 | Histidine decarboxylase | Aeromonas salmonicida | modified codon usage for |
| | enzyme T0QL99 | | (EC 4.1.1.22) (Fragment) | subsp. pectinolytica 34mel | Corynebacterium glutamicum and |
| | | | | | Saccharomyces cerevisiae |
| 106 | DNA seq | A0A1B8 | Histidine decarboxylase | Morganella psychrotolerans | modified codon usage for |
| | for enzyme | HLR1 | (HDC) (EC 4.1.1.22) | | Corynebacterium glutamicum and |
| | A0A1B8HLR1 | | | | Saccharomyces cerevisiae |
| 107 | DNA seq2 for | Q9Z472 | ATP phosphoribosyltransferase | Corynebacterium glutamicum | modified codon usage for |
| | enzyme Q9Z472 | | (ATP-PRT) (ATP-PRTase) | (strain ATCC 13032/ | Corynebacterium glutamicum and |
| | | | | DSM 20300/JCM 1318/ | Saccharomyces cerevisiae |
| | | | | LMG 3730/NCIMB 10025) | |
| 108 | DNA seq for | P0A717 | Ribose-phosphate | Escherichia coli (strain K12) | modified codon usage for |
| | enzyme P0A717 | | pyrophosphokinase | | Corynebacterium glutamicum and |
| | | | (RPPK) (EC 2.7.6.1) | | Saccharomyces cerevisiae |
| | | | (5-phospho-D-ribosyl | | |
| | | | alpha-1-diphosphate) | | |
| | | | (Phosphoribosyl | | |
| | | | diphosphate synthase) | | |
| | | | (Phosphoribosyl | | |
| | | | pyrophosphate synthase) | | |
| | | | (P-Rib-PP synthase) | | |
| | | | (PRPP synthase) (PRPPase) | | |
| 109 | DNA seq for | Q12265 | Ribose-phosphate | Saccharomyces cerevisiae | modified codon usage for |
| | enzyme Q12265 | | pyrophosphokinase 5 | (strain ATCC 204508/S288c) | Corynebacterium glutamicum and |
| | | | (EC 2.7.6.1) (Phosphoribosyl | (Baker's yeast) | Saccharomyces cerevisiae |
| | | | pyrophosphate synthase 5) | | |
| 110 | DNA seq for | P32895 | Ribose-phosphate | Saccharomyces cerevisiae | modified codon usage for |
| | enzyme P32895 | | pyrophosphokinase 1 | (strain ATCC 204508/S288c) | Corynebacterium glutamicum and |
| | | | (EC 2.7.6.1) (Phosphoribosyl | (Baker's yeast) | Saccharomyces cerevisiae |
| | | | pyrophosphate synthase 1) | | |
| 111 | DNA seq for | P23254 | Transketolase | Saccharomyces cerevisiae | modified codon usage for |
| | enzyme P23254 | | | (strain ATCC 204508/S288c) | Corynebacterium glutamicum and |
| | | | | (Baker's yeast) | Saccharomyces cerevisiae |
| 112 | DNA seq for | P06775 | Histidine permease | Saccharomyces cerevisiae | modified codon usage for |
| | enzyme P06775 | | | (strain ATCC 204508/S288c) | Corynebacterium glutamicum and |
| | | | | (Baker's yeast) | Saccharomyces cerevisiae |
| 113 | DNA seq2 for | P00815 | trifunctional histidinol | Saccharomyces cerevisiae | modified codon usage for |
| | enzyme P00815 | | dehydrogenase/ | (strain ATCC 204508/S288c) | Corynebacterium glutamicum and |
| | | | phosphoribosyl-AMP | (Baker's yeast) | Saccharomyces cerevisiae |
| | | | cyclohydrolase/ | | |
| | | | phosphoribosyl-ATP | | |
| | | | diphosphatase | | |
| 114 | DNA seq2 fo | P07172 | Histidinol-phosphate | Saccharomyces cerevisiae | modified codon usage for |
| | enzyme P07172 | | aminotransferase | (strain ATCC 204508/S288c) | Corynebacterium glutamicum and |
| | | | | (Baker's yeast) | Saccharomyces cerevisiae |
| 115 | DNA seq6 for | P00498 | ATP phosphoribosyltransferase | Saccharomyces cerevisiae | modified codon usage for |
| | enzyme P00498 | | (ATP-PRT) (ATP-PRTase) | (strain ATCC 204508/S288c) | Corynebacterium glutamicum and |
| | | | | (Baker's yeast) | Saccharomyces cerevisiae |
| 116 | DNA seq for | P40545 | 1-(5-phosphoribosyl)-5-[(5- | Saccharomyces cerevisiae | modified codon usage for |
| | enzyme P40545 | | phosphoribosylamino) | (strain ATCC 204508/S288c) | Corynebacterium glutamicum and |
| | | | methylideneamino] | (Baker's yeast) | Saccharomyces cerevisiae |
| | | | imidazole-4-carboxamide | | |
| | | | isomerase (EC 5.3.1.16) | | |
| | | | (5-proFAR isomerase) | | |
| | | | (Phosphoribosylformimino- | | |
| | | | 5-aminoimidazole | | |
| | | | carboxamide | | |
| | | | ribotide isomerase) | | |
| 117 | DNA seq2 for | P33734 | Imidazole | Saccharomyces cerevisiae | modified codon usage for |
| | enzyme P33734 | | glycerol phosphate | (strain ATCC 204508/S288c) | Corynebacterium glutamicum and |
| | | | synthase hisHF | (Baker's yeast) | Saccharomyces cerevisiae |
| 118 | DNA seq for | P00942 | Triosephosphate isomerase | Saccharomyces cerevisiae | modified codon usage for |
| | enzyme P00942 with | | (TIM) (EC 5.3.1.1) | (strain ATCC 204508/S288c) | Corynebacterium glutamicum and |
| | substitution 1170V | | (Triose-phosphate isomerase) | (Baker's yeast) | Saccharomyces cerevisiae |
| 119 | DNA seq3 for | Q9Z472 | ATP phosphoribosyltransferase | Corynebacterium glutamicum | modified codon usage for |
| | enzyme Q9Z472 | | (ATP-PRT) (ATP-PRTase) | (strain ATCC 13032/ | Corynebacterium glutamicum and |
| | | | (EC 2.4.2.17) | DSM 20300/JCM 1318/ | Saccharomyces cerevisiae |
| 120 | DNA seq5 | Q9Z472 | ATP phosphoribosyltransferase | LMG 3730/NCIMB 10025) | modified codon usage for |
| | for enzyme Q9Z472 | | (ATP-PRT) (ATP-PRTase) | (strain ATCC 13032/ | Corynebacterium glutamicum and |
| | with substitution | | (EC 2.4.2.17) | DSM 20300/JCM 1318/ | Saccharomyces cerevisiae |
| | N215K, L231F, T235A | | | LMG 3730/NCIMB 10025) | |
| 121 | DNA seq for | O66000 | Histidine decarboxylase | Oenococcus oeni | modified codon usage for |
| | enzyme O66000 | | proenzyme | (Leuconostoc oenos) | Corynebacterium glutamicum and |
| | | | | | Saccharomyces cerevisiae |
| 122 | DNA seq for enzyme | A0A0R1 | Pyruvoyl family | Lactobacillus aviarius | modified codon usage for |
| | A0A0R1Y874 | Y874 | histidine decarboxylase | subsp. aviarius DSM 20655 | Corynebacterium glutamicum and |
| | | | | | Saccharomyces cerevisiae |
| 123 | DNA seq for enzyme | A0A1H1 | Histidine decarboxylase | Pseudomonas sp. bs2935 | modified codon usage for |
| | A0A1H1TEB8 with | TEB8 | (HDC) (EC 4.1.1.22) | | Corynebacterium glutamicum and |
| | substitution S9R | | | | Saccharomyces cerevisiae |
| 124 | DNA seq for enzyme | A0A089 | Histidine decarboxylase | Pseudomonas rhizosphaerae | modified codon usage for |
| | A0A089YPE5 | YPE5 | (HDC) (EC 4.1.1.22) | | Corynebacterium glutamicum and |
| | | | | | Saccharomyces cerevisiae |
| 125 | DNA seq for enzyme | A0A126 | Histidine decarboxylase | Pseudomonas putida | modified codon usage for |
| | A0A12656G9 | 56G9 | (HDC) (EC 4.1.1.22) | (Arthrobacter siderocapsulatus) | Corynebacterium glutamicum and |
| | | | | | Saccharomyces cerevisiae |
| 126 | DNA seq4 for enzyme | A0A0J6K | Histidine decarboxylase | Chromobacterium sp. LK1 | modified codon usage for |
| | A0A0J6KM89 | M89 | (HDC) (EC 4.1.1.22) | | Corynebacterium glutamicum and |
| | | | | | Saccharomyces cerevisiae |
| 127 | DNA seq for enzyme | A0A0A1 | Histidine decarboxylase | Citrobacter pasteurii | modified codon usage for |
| | A0A0A1R6V3 | R6V3 | (HDC) (EC 4.1.1.22) | | Corynebacterium glutamicum and |
| | | | | | Saccharomyces cerevisiae |
| 128 | DNA seq for enzyme | A0A1W0 | Histidine decarboxylase | Chromobacterium haemolyticum | modified codon usage for |
| | A0A1W0CM88 | CM88 | (HDC) (EC 4.1.1.22) | | Corynebacterium glutamicum and |
| | | | Histidine decarboxylase | | Saccharomyces cerevisiae |
| 129 | DNA seq6 for | P00862 | proenzyme | Lactobacillus sp. (strain 30a) | modified codon usage for |
| | enzyme P00862 | | (EC 4.1.1.22) (Pi chain) | | Corynebacterium glutamicum and |
| | | | [Cleaved into: Histidine | | Saccharomyces cerevisiae |
| | | | decarboxylase beta chain; | | |
| | | | Histidine decarboxylase | | |
| | | | alpha chain] | | |
| 130 | DNA seq for enzyme | A0A0K6 | Histidine decarboxylase | Lactobacillus reuteri | modified codon usage for |
| | A0A0K6GJ74 | GJ74 | proenzyme | | Corynebacterium glutamicum and |
| | | | | | Saccharomyces cerevisiae |
| 131 | DNA seq for | P38620 | Ribose-phosphate | Saccharomyces cerevisiae | modified codon usage for |
| | enzyme P38620 | | pyrophosphokinase 2 | (strain ATCC 204508/S288c) | Corynebacterium glutamicum and |
| | | | (EC 2.7.6.1) (Phosphoribosyl | (Baker's yeast) | Saccharomyces cerevisiae |
| | | | pyrophosphate synthase 2) | | |
| 132 | DNA seq for | P38689 | Ribose-phosphate | Saccharomyces cerevisiae | modified codon usage for |
| | enzyme P38689 | | pyrophosphokinase 3 | (strain ATCC 204508/S288c) | Corynebacterium glutamicum and |
| | | | (EC 2.7.6.1) (Phosphoribosyl | (Baker's yeast) | Saccharomyces cerevisiae |
| | | | pyrophosphate synthase 3) | | |
| 133 | DNA seq for | P15019 | Transaldolase (EC 2.2.1.2) | Saccharomyces cerevisiae | modified codon usage for |
| | enzyme P15019 | | | (strain ATCC 204508/S288c) | Corynebacterium glutamicum and |
| | | | | (Baker's yeast) | Saccharomyces cerevisiae |
| 134 | DNA seq for | Q680A5 | Ribose-phosphate | Arabidopsis thaliana | modified codon usage for |
| | enzyme Q680A5 | | pyrophosphokinase 4 | (Mouse-ear cress) | Corynebacterium glutamicum and |
| | | | (EC 2.7.6.1) (Phosphoribosyl | | Saccharomyces cerevisiae |
| | | | pyrophosphate synthase 4) | | |
| 135 | DNA seq for | O59667 | Histidine biosynthesis | Schizosaccharomyces pombe | modified codon usage for |
| | enzyme O59667 | | bifunctional | (strain 972/ATCC 24843) | Corynebacterium glutamicum and |
| | | | protein his7 [Includes: | (Fission yeast) | Saccharomyces cerevisiae |
| | | | Phosphoribosyl-AMP | | |
| | | | cyclohydrolase (EC 3.5.4.19); | | |
| | | | Phosphoribosyl-ATP | | |
| | | | pyrophosphatase | | |
| | | | (EC 3.6.1.31)] | | |
| 136 | DNA seq2 for | P38635 | Histidinol-phosphatase | Saccharomyces cerevisiae | modified codon usage for |
| | enzyme P38635 | | (HolPase) (EC 3.1.3.15) | (strain ATCC 204508/S288c) | Corynebacterium glutamicum and |
| | | | | (Baker's yeast) | Saccharomyces cerevisiae |
| 137 | DNA seq for | A4QEF2 | Glucose-6-phosphate | Corynebacterium glutamicum | modified codon usage for |
| | enzyme | | 1-dehydrogenase | (strain R) | Corynebacterium glutamicum and |
| | A4QEF2 with | | (G6PD) (EC 1.1.1.49) | | Saccharomyces cerevisiae |
| | substitution A243T | | | | |
| 138 | DNA seq7 for | P00862 | Histidine decarboxylase | Lactobacillus sp. (strain 30a) | Yarrowia lipolytica |
| | enzyme P00862 | | proenzyme | | |
| 139 | DNA seq5 | A0A0J6K | Histidine decarboxylase | Chromobacterium sp. LK1 | modified codon usage for |
| | for enzyme | M89 | | | Corynebacterium glutamicum and |
| | A0A0J6KM89 | | | | Saccharomyces cerevisiae |
| 140 | DNA seq6 | Q9Z472 | ATP phosphoribosyl transferase | Corynebacterium glutamicum | modified codon usage for |
| | for enzyme Q9Z472 | | | ATCC 13032 | Corynebacterium glutamicum and |
| | with substitution | | | | Saccharomyces cerevisiae |
| | N215K, L231F, T235A | | | | |
| 141 | DNA seq4 for | Q9Z472 | ATP phosphoribosyltransferase | Corynebacterium glutamicum | Saccharomyces cerevisiae |
| | enzyme Q9Z472 | | | (strain ATCC 13032/ | |
| | | | | DSM 20300/JCM 1318/ | |
| | | | | LMG 3730/NCIMB 10025) | |
| 142 | AA seq for | | | | |
| | N-terminal | | | | |
| | solubility tag | | | | |
| 143 | AA seq for | E7EAU9 | Ribose-phosphate | Bacillus amyloliquefaciens | Yarrowia lypolytica |
| | enzyme E7EAU9 | | pyrophosphokinase (RPPK) | | |

### REFERENCES

1. Gezginc, Y., et al., Biogenic amines formation in Streptococcus thermophilus isolated from home-made natural yogurt. Food Chem, 2013. 138(1): p. 655-62.
2. Byun, B. Y. and J. H. Mah, Occurrence of biogenic amines in Miso, Japanese traditional fermented soybean paste. J Food Sci, 2012. 77(12): p. T216-23.
3. Landete, J. M., et al., Molecular methods for the detection of biogenic amine-producing bacteria on foods. Int J Food Microbiol, 2007. 117(3): p. 258-69.
4. Ferstl, R., et al., Histamine receptor 2 is a key influence in immune responses to intestinal histamine-secreting microbes. J Allergy Clin Immunol, 2014. 134(3): p. 744-746 e3.
5. Tabanelli, G., et al., Effect of chemico-physical parameters on the histidine decarboxylase (HdcA) enzymatic activity in Streptococcus thermophilus PRI60. J Food Sci, 2012. 77(4): p. M231-7.
6. Wauters, G., et al., Histidine decarboxylase in Enterobacteriaceae revisited. J Clin Microbiol, 2004. 42(12): p. 5923-4.
7. Lee, M. E., et al., A Highly Characterized Yeast Toolkit for Modular, Multipart Assembly. ACS Synth Biol, 2015. 4(9): p. 975-86.
8. Roland, B. P., et al., Triosephosphate isomerase 1170V alters catalytic site, enhances stability and induces pathology in a Drosophila model of TPI deficiency. Biochim Biophys Acta, 2015. 1852(1): p. 61-9.

1. An engineered microbial cell that comprises a non-native histidine decarboxylase, wherein the non-native histidine decarboxylase comprises at least 70% amino acid sequence identity with a histidine decarboxylase having SEQ ID NO:1 or SEQ ID NO:4, wherein the engineered microbial cell produces histamine in culture.

2. The engineered microbial cell of claim 1, additionally comprising an ATP phosphoribosyltransferase that is at least 70% identical to SEQ ID NO:3 or SEQ ID NO:5 or an imidazoleglycerol-phosphate dehydratase that is at least 70% identical to SEQ ID NO:2.

3. The engineered microbial cell of claim 2, wherein the engineered microbial cell comprises increased activity of one or more upstream histamine pathway enzyme(s) selected from the group consisting of an ATP phosphoribosyltransferase, a phosphoribosyl-ATP pyrophosphatase, a phosphoribosyl-AMP cyclohydrolase, a 5′ProFAR isomerase, an imidazole-glycerol phosphate synthase, an imidazole-glycerol phosphate dehydratase, a histidinol-phosphate aminotransferase, a histidinol-phosphate phosphatase, histidinol dehydrogenase, and a ribose phosphate pyrophosphokinase, said increased activity being increased relative to a control cell.

4. The engineered microbial cell of claim 1, wherein the engineered microbial cell comprises reduced activity of one or more enzyme(s) that consume one or more histamine pathway precursors, said reduced activity being reduced relative to a control cell.

5. The engineered microbial cell of claim 1, wherein the engineered microbial cell additionally comprises a feedback-deregulated glucose-6-phosphate dehydrogenase or a feedback-deregulated ATP phosphoribosyltransferase.

6-11. (canceled)

12. A culture comprising the engineered microbial cell according to claim 1.

13. (canceled)

14. A method for producing histamine, the method comprising culturing the engineered microbial cell of claim 1 under conditions suitable for producing histamine, thereby producing histamine.

15. The method for producing histamine of claim 14, wherein the histamine is released into the culture medium; and
the method additionally comprises isolating histamine from the culture medium.

16. The engineered microbial cell of claim 2, which comprises:
a non-native histidine decarboxylase comprising at least 70% amino acid sequence identity with a histidine decarboxylase having SEQ ID NO:1; and
an ATP phosphoribosyltransferase comprising at least 70% amino acid sequence identity with an ATP phosphoribosyltransferase having SEQ ID NO:3.

17. The engineered microbial cell of claim 16, wherein the engineered microbial cell is a Corynebacteria glutamicum cell.

18. The engineered microbial cell of claim 17, which produces histamine at a level of not more than 10 gm/L of culture medium in culture.

19. The engineered microbial cell of claim 2, which comprises:
a non-native histidine decarboxylase comprising at least 70% amino acid sequence identity with a histidine decarboxylase SEQ ID NO:1; and
an imidazoleglycerol-phosphate dehydratase comprising at least 70% amino acid sequence identity with an imidazoleglycerol-phosphate dehydratase having SEQ ID NO:2.

20. The engineered microbial cell of claim 19, wherein the engineered microbial cell is a Corynebacteria glutamicum cell.

21. The engineered microbial cell of claim 20, which produces histamine at a level of not more than 10 gm/L of culture medium in culture.

22. The engineered microbial cell of claim 2, which comprises:
a non-native histidine decarboxylase comprising at least 70% amino acid sequence identity with a histidine decarboxylase having SEQ ID NO:4; and
an ATP phosphoribosyltransferase having SEQ ID NO:5.

23. The engineered microbial cell of claim 22, wherein the engineered microbial cell is a Bacillus subtilis cell.

24. The engineered microbial cell of claim 23, which produces histamine at a level of not more than 10 gm/L of culture medium in culture.

25. The engineered microbial cell of claim 1, wherein the non-native histidine decarboxylase comprises at least 90% amino acid sequence identity with a histidine decarboxylase having SEQ ID NO:1 or SEQ ID NO:4.

26. The engineered microbial cell of claim 2, wherein the ATP phosphoribosyltransferase comprises at least 90% amino acid sequence identity with an ATP phosphoribosyltransferase having SEQ ID NO:3 or SEQ ID NO:5, or the imidazoleglycerol-phosphate dehydratase comprises at least 90% amino acid sequence identity with an imidazoleglycerol-phosphate dehydratase having SEQ ID NO:2

27. The engineered microbial cell of claim 16, which produces histamine at a level of at least 50 mg/L of culture medium in culture.

28. The engineered microbial cell of claim 19, which produces histamine at a level of at least 20 mg/L of culture medium in culture.

29. The engineered microbial cell of claim 22, which produces histamine at a level of at least 10 mg/L of culture medium in culture.
